(19) United States
(12) Patent Application Publication
(10) Pub. No.: US 2024/0336556 A1
(43) Pub. Date: Oct. 10, 2024

(54) R-KETAMINE SALTS AND METHODS OF USE THEREOF

(71) Applicant: Perception Neuroscience, Inc., New York, NY (US)

(72) Inventors: Mouhannad JUMAA, San Jose, CA (US); Terence Alfred KELLY, Ridgefield, CT (US)

(21) Appl. No.: 18/700,013

(22) PCT Filed: Oct. 12, 2022
(86) PCT No.: PCT/US2022/046412

§ 371 Date: Apr. 10, 2024

Related U.S. Application Data
(60) Provisional application No. 63/254,878, filed on Oct. 12, 2021.

(51) Int. Cl. C07C 225/20; A61K 31/135; C07C 55/06; C07C 55/10; C07C 57/15; C07C 59/255; C07C 59/265; C07D 275/06

(52) U.S. Cl. CPC C07C 225/20; A61K 31/135; C07C 55/06; C07C 55/10; C07C 57/15; C07C 59/255; C07C 59/265; C07D 275/06; C07B 2200/13; C07C 2601/14

(57) ABSTRACT

The present disclosure relates to novel salts and salt forms of the (2R)-2-(2-chlorophenyl)-2-(methylamino)cyclohexan-1-one (R-ketamine) and to processes for their preparation. The disclosure is also directed to pharmaceutical compositions containing at least one R-ketamine salt or salt form and to the therapeutic and/or prophylactic use of such salts, salt forms, and compositions thereof.

### CROSS REFERENCE TO RELATED APPLICATIONS

This application claims priority to and the benefit of U.S. Provisional Application No. 63/254,878, filed on Oct. 12, 2021, which is incorporated by reference herein in its entirety for all purposes.

### FIELD OF THE INVENTION

The present disclosure relates to salts and salt forms of (2R)-2-(2-chlorophenyl)-2-(methylamino)cyclohexan-1-one (R-ketamine), processes for their preparation and their use in the manufacture of a medicament for treating patients. The disclosure is also directed to pharmaceutical compositions containing at least one R-ketamine salt and to the therapeutic and/or prophylactic use of such salts and compositions.

### BACKGROUND

R-ketamine has been shown to be active in the treatment of various neurological conditions and in alleviating corresponding symptoms. Optimizing the manufacturing of R-ketamine for preparing a pharmaceutical composition requires the development of new, stable and useful salts and polymorphs thereof.

### SUMMARY

The present disclosure is directed to new salts and salt forms of R-ketamine for use in the manufacture of a medicament or pharmaceutical composition for the treatment of patients in need thereof.

In one aspect, the present disclosure is directed to an R-ketamine saccharin salt.

In one embodiment, the R-ketamine saccharin salt is crystalline.

In one embodiment, the R-ketamine saccharin salt is amorphous.

In one embodiment, the R-ketamine saccharin salt is a crystalline polymorphic form characterized by PXRD peaks at two or more, or three peaks selected from the group consisting of 13.8 °2θ, 15.6 °2θ, and 23.5 °2θ (±0.2 °2θ; ±0.1 °2θ; or ±0.0 °2θ; Cu Kα1 radiation).

In one embodiment, the R-ketamine saccharin salt is a crystalline polymorphic form characterized by PXRD peaks at 13.8 °2θ, 15.6 °2θ, and 23.5 °2θ (±0.2 °2θ; ±0.1 °2θ; or ±0.0 °2θ; Cu Kα1 radiation).

In one embodiment, the R-ketamine saccharin salt is a crystalline polymorphic form characterized by a PXRD spectrum substantially similar to that shown in FIG. 9.

In one embodiment, the R-ketamine saccharin salt is a crystalline polymorphic form characterized by one, two, three, four, five, six, seven, eight, nine, ten, eleven, twelve, thirteen, fourteen, fifteen or sixteen PXRD peaks selected from those set forth in Table 2.

In one embodiment, the R-ketamine saccharin salt is a 1:1 R-ketamine:saccharin salt.

In one embodiment, the R-ketamine saccharin salt is a 2:1 R-ketamine:saccharin salt.

In one embodiment, the R-ketamine saccharin salt is a 1:2 R-ketamine:saccharin salt.

In one aspect, the present disclosure is directed to an R-ketamine fumarate salt.

In one embodiment, the R-ketamine fumarate salt is crystalline.

In one embodiment, the R-ketamine fumarate salt is a crystalline polymorphic form Form A.

In one embodiment, the R-ketamine fumarate salt Form A is characterized by PXRD peaks at two or more, or three peaks selected from the group consisting of 11.6 °2θ, 13.4 °2θ, and 14.6 °2θ (±0.2 °2θ; ±0.1 °2θ; or ±0.0 °2θ; Cu Kα1 radiation).

In one embodiment, the R-ketamine fumarate salt Form A is characterized by PXRD peaks at 11.6 °2θ, 13.4 °2θ, and 14.6 °2θ (±0.2 °2θ; ±0.1 °2θ; or ±0.0 °2θ; Cu Kα1 radiation).

In one embodiment, the R-ketamine fumarate salt Form A is characterized by a PXRD spectrum substantially similar to that shown in FIG. 21.

In one embodiment, the R-ketamine fumarate salt Form A is characterized by one, two, three, four, five, six, seven, eight, nine, ten, eleven, twelve, thirteen, fourteen, fifteen or sixteen PXRD peaks selected from those set forth in Table 3.

In one embodiment, the R-ketamine fumarate salt is a crystalline polymorphic form Form B.

In one embodiment, the R-ketamine fumarate salt Form B is characterized by PXRD peaks at two or more, or three peaks selected from the group consisting of 14.5 °2θ, 15.1 °2θ, and 20.4 °2θ (±0.2 °2θ; ±0.1 °2θ; or ±0.0 °2θ; Cu Kα1 radiation).

In one embodiment, the R-ketamine fumarate salt Form B is characterized by PXRD peaks at 14.5 °2θ, 15.1 °2θ, and 20.4 °2θ (±0.2 °2θ; ±0.1 °2θ; or ±0.0 °2θ; Cu Kα1 radiation).

In one embodiment, the R-ketamine fumarate salt Form B is characterized by a PXRD spectrum substantially similar to that shown in FIG. 68.

In one embodiment, the R-ketamine fumarate salt Form B is characterized by one, two, three, four, five, six, seven, eight, nine, ten, eleven, twelve, thirteen, fourteen, fifteen or sixteen PXRD peaks selected from those set forth in Table 4.

In one embodiment, the R-ketamine fumarate salt is a 1:1 R-ketamine:fumarate salt.

In one embodiment, the R-ketamine fumarate salt is a 2:1 R-ketamine:fumarate salt.

In one embodiment, the R-ketamine fumarate salt is between a 1:1 and 1:2 R-ketamine:fumarate salt.

In one embodiment, the R-ketamine fumarate salt is a hemifumarate salt. In one embodiment, the R-ketamine fumarate salt is a Form A hemifumarate salt. In one embodiment, the R-ketamine fumarate salt is a Form B hemifumarate salt.

In one aspect, the present disclosure is directed to an R-ketamine succinate salt.

In one embodiment, the R-ketamine succinate salt is crystalline.

In one embodiment, the R-ketamine succinate salt is a crystalline polymorphic form characterized by PXRD peaks at two or more, or three peaks selected from the group consisting of 9.0 °2θ, 13.5 °2θ, and 18.1 °2θ (±0.2 °2θ; ±0.1 °2θ; or 10.0 °2θ; Cu Kα1 radiation).

In one embodiment, the R-ketamine succinate salt is a crystalline polymorphic form characterized by PXRD peaks at 9.0 °2θ, 13.5 °2θ, and 18.1 °2θ (±0.2 °2θ; ±0.1 °2θ; or ±0.0 °2θ; Cu Kα1 radiation).

In one embodiment, the R-ketamine succinate salt a crystalline polymorphic form characterized by a PXRD spectrum substantially similar to that shown in FIG. 29.

In one embodiment, the R-ketamine succinate salt is a crystalline polymorphic form characterized by one, two, three, four, five, six, seven, eight, nine, ten, eleven, twelve, thirteen, fourteen, fifteen or sixteen PXRD peaks selected from those set forth in Table 5.

In one aspect, the present disclosure is directed to an R-ketamine sulfate salt.

In one embodiment, the R-ketamine sulfate salt is crystalline.

In one embodiment, the R-ketamine sulfate salt is a crystalline polymorphic form characterized by PXRD peaks at two or more, or three peaks selected from the group consisting of 19.8 °2θ, 22.5 °2θ, and 26.1 °2θ (±0.2 °2θ; ±0.1 °2θ; or ±0.0 °2θ; Cu Kα1 radiation).

In one embodiment, the R-ketamine sulfate salt is a crystalline polymorphic form characterized by PXRD peaks at 19.8 °2θ, 22.5 °2θ, and 26.1 °2θ (±0.2 °2θ; ±0.1 °2θ; or ±0.0 °2θ; Cu Kα1 radiation).

In one embodiment, the R-ketamine sulfate salt is a crystalline polymorphic form characterized by PXRD spectrum substantially similar to that shown in FIG. 36.

In one embodiment, the R-ketamine sulfate salt is a crystalline polymorphic form characterized by one, two, three, four, five, six, seven, eight, nine, or ten PXRD peaks selected from those set forth in Table 6.

In one aspect, the present disclosure is directed to an R-ketamine D-tartrate salt.

In one embodiment, the R-ketamine D-tartrate salt is crystalline. In one embodiment, the R-ketamine D-tartrate salt is non-solvated.

In one embodiment, the R-ketamine D-tartrate salt is a crystalline polymorphic form Form A.

In one embodiment, the R-ketamine D-tartrate salt Form A is characterized by PXRD peaks at two or more, or three peaks selected from the group consisting of 10.3 °2θ, 14.7 °2θ, and 15.4 °2θ (±0.2 °2θ; ±0.1 °2θ; or ±0.0 °2θ; Cu Kα1 radiation).

In one embodiment, the R-ketamine D-tartrate salt Form A is characterized by PXRD peaks at 10.3 °2θ, 14.7 °2θ, and 15.4 °2θ (±0.2 °2θ; ±0.1 °2θ; or ±0.0 °2θ; Cu Kα1 radiation).

In one embodiment, the R-ketamine D-tartrate salt Form A is characterized by a PXRD spectrum substantially similar to that shown in FIG. 40.

In one embodiment, the R-ketamine D-tartrate salt Form A is characterized by one, two, three, four, five, six, seven, eight, nine, ten, eleven, twelve, thirteen, fourteen, fifteen or sixteen PXRD peaks selected from those set forth in Table 7.

In one embodiment, the R-ketamine D-tartrate salt is a crystalline polymorphic form Form B.

In one embodiment, the R-ketamine D-tartrate salt Form B is characterized by PXRD peaks at two or more, or three peaks selected from the group consisting of 5.9 °2θ, 12.7 °2θ, and 14.7 °2θ (±0.2 °2θ; ±0.1 °2θ; or ±0.0 °2θ; Cu Kα1 radiation).

In one embodiment, the R-ketamine D-tartrate salt Form B is characterized by PXRD peaks at 5.9 °2θ, 12.7 °2θ, and 14.7 °2θ (±0.2 °2θ; ±0.1 °2θ; or ±0.0 °2θ; Cu Kα1 radiation).

In one embodiment, the R-ketamine D-tartrate salt Form B is characterized by a PXRD spectrum substantially similar to that shown in FIG. 56.

In one embodiment, the R-ketamine D-tartrate salt Form B is characterized by one, two, three, four, five, six, seven, eight, nine, ten, eleven, twelve, thirteen, fourteen, fifteen or sixteen PXRD peaks selected from those set forth in Table 8.

In one embodiment, the R-ketamine D-tartrate salt is a crystalline polymorphic form Form C.

In one embodiment, the R-ketamine D-tartrate salt Form C is characterized by PXRD peaks at two or more, or three peaks selected from the group consisting 6.1 °2θ, 11.0 °2θ, and 12.2 °2θ (±0.2 °2θ; ±0.1 °2θ; or +0.0 °2θ; Cu Kα1 radiation).

In one embodiment, the R-ketamine D-tartrate salt Form C is characterized by PXRD peaks at 6.1 °2θ, 11.0 °2θ, and 12.2 °2θ (±0.2 °2θ; ±0.1 °2θ; or ±0.0 °2θ; Cu Kα1 radiation).

In one embodiment, the R-ketamine D-tartrate salt Form C is characterized by PXRD peaks at two or more, or three peaks selected from the group consisting 11.0 °2θ, 13.6 °2θ, and 14.0 °2θ (±0.2 °2θ; ±0.1 °2θ; or ±0.0 °2θ; Cu Kα1 radiation).

In one embodiment, the R-ketamine D-tartrate salt Form C is characterized by PXRD peaks at 11.0 °2θ, 13.6 °2θ, and 14.0 °2θ (±0.2 °2θ; ±0.1 °2θ; or ±0.0 °2θ; Cu Kα1 radiation).

In one embodiment, the R-ketamine D-tartrate salt Form C is characterized by a PXRD spectrum substantially similar to that shown in FIG. 61.

In one embodiment, the R-ketamine D-tartrate salt Form C is characterized by a PXRD spectrum substantially similar to those shown in FIG. 94.

In one embodiment, the R-ketamine D-tartrate salt Form C is characterized by one, two, three, four, five, six, seven, eight, nine, ten, eleven, twelve, thirteen, fourteen, fifteen or sixteen PXRD peaks selected from those set forth in Table 9.

In one embodiment, the R-ketamine D-tartrate salt is a crystalline polymorphic form Form D.

In one embodiment, the R-ketamine D-tartrate salt Form D is characterized by PXRD peaks at two or more, or three peaks selected from the group consisting 12.7 °2θ, 14.3 °2θ, and 19.8 °2θ (±0.2 °2θ; ±0.1 °2θ; or ±0.0 °2θ; Cu Kα1 radiation).

In one embodiment, the R-ketamine D-tartrate salt Form D is characterized by PXRD peaks at 12.7 °2θ, 14.3 °2θ, and 19.8 °2θ (±0.2 °2θ; ±0.1 °2θ; or ±0.0 °2θ; Cu Kα1 radiation).

In one embodiment, the R-ketamine D-tartrate salt Form D is characterized by a PXRD spectrum substantially similar to that shown in FIG. 87.

In one embodiment, the R-ketamine D-tartrate salt Form D is characterized by one, two, three, four, five, six, seven, eight, nine, ten, eleven, twelve, thirteen, fourteen, fifteen or sixteen PXRD peaks selected from those set forth in Table 10.

In one embodiment, the R-ketamine D-tartrate salt Form A is a 1:1 R-ketamine:D-tartrate salt. In one embodiment, the R-ketamine D-tartrate salt Form B is a 1:1 R-ketamine:D-tartrate salt. In one embodiment, the R-ketamine D-tartrate salt Form C is a 1:1 R-ketamine:D-tartrate salt. In one embodiment, the R-ketamine D-tartrate salt Form D is a 1:1 R-ketamine:D-tartrate salt. In one embodiment, the R-ketamine D-tartrate salt Form A is a 2:1 R-ketamine:D-tartrate salt. In one embodiment, the R-ketamine D-tartrate salt Form B is a 2:1 R-ketamine:D-tartrate salt. In one embodiment, the R-ketamine D-tartrate salt Form C is a 2:1 R-ketamine:D-tartrate salt. In one embodiment, the R-ketamine D-tartrate salt Form D is a 2:1 R-ketamine:D-tartrate salt. In one embodiment, the R-ketamine D-tartrate salt Form A is between a 1:1 and a 2:1 R-ketamine:D-tartrate salt. In one embodiment, the R-ketamine D-tartrate salt Form B is between a 1:1 and a 2:1 R-ketamine:D-tartrate salt. In one embodiment, the R-ketamine D-tartrate salt Form C is between a 1:1 and a 2:1 R-ketamine:D-tartrate salt. In one embodiment, the R-ketamine D-tartrate salt Form D is between a 1:1 and a 2:1 R-ketamine:D-tartrate salt.

In one aspect, the present disclosure is directed to an R-ketamine oxalate salt.

In one embodiment, the R-ketamine oxalate salt is crystalline.

In one embodiment, the R-ketamine oxalate salt is a crystalline polymorphic form characterized by PXRD peaks at two or more, or three peaks selected from the group consisting of 12.8 °2θ, 14.8 °2θ and 16.2 °2θ (10.2 °2θ; 0.1 °2θ; or 0.0 °2θ; Cu Kα1 radiation).

In one embodiment, the R-ketamine oxalate salt is a crystalline polymorphic form characterized by PXRD peaks at 12.8 °2θ, 14.8 °2θ and 16.2 °2θ (±0.2 °2θ; 0.1 °2θ; or ±0.0 °2θ; Cu Kα1 radiation).

In one embodiment, the R-ketamine oxalate salt is a crystalline polymorphic form characterized by a PXRD spectrum substantially similar to that shown in FIG. 43.

In one embodiment, the R-ketamine oxalate salt is a crystalline polymorphic form characterized by one, two, three, four, five, six, seven, eight, nine, ten, eleven, twelve, thirteen, fourteen, fifteen or sixteen PXRD peaks selected from those set forth in Table 11.

In one embodiment, the R-ketamine oxalate salt is a 2:1 R-ketamine:oxalate salt.

In one aspect, the present disclosure is directed to an R-ketamine citrate salt.

In one embodiment, the R-ketamine citrate salt is crystalline.

In one embodiment, the R-ketamine citrate salt is a crystalline polymorphic form characterized by PXRD peaks at two or more, or three peaks selected from the group consisting of 14.8 °2θ, 16.8 °2θ, and 21.4 °2θ (±0.2 °2θ; ±0.1 °2θ; or ±0.0 °2θ; Cu Kα1 radiation).

In one embodiment, the R-ketamine citrate salt is a crystalline polymorphic form characterized by PXRD peaks at 14.8 °2θ, 16.8 °2θ, and 21.4 °2θ (±0.2 °2θ; ±0.1 °2θ; or ±0.0 °2θ; Cu Kα1 radiation).

In one embodiment, the R-ketamine citrate salt is a crystalline polymorphic form characterized by a PXRD spectrum substantially similar to that shown in FIG. 46.

In one embodiment, the R-ketamine citrate salt is a crystalline polymorphic form characterized by one, two, three, four, five, six, seven, eight, nine, ten, eleven, twelve, thirteen, fourteen, fifteen or sixteen PXRD peaks selected from those set forth in Table 12.

In one embodiment, the R-ketamine citrate salt is a 1:1 R-ketamine:citrate salt.

In one embodiment, the R-ketamine citrate salt is a 2:1 R-ketamine:citrate salt.

In one embodiment, the R-ketamine citrate salt is between a 1:1 and a 3:1 R-ketamine:citrate salt.

In one aspect, the present disclosure is directed to R-ketamine free base Form A.

In one embodiment, the R-ketamine free base Form A is crystalline.

In one embodiment, the R-ketamine free base Form A is a crystalline polymorphic form characterized by PXRD peaks at two or more, or three peaks selected from the group consisting of 14.8 °2θ, 20.7 °2θ, and 23.8 °2θ (±0.2 °2θ; ±0.1 °2θ; or ±0.0 °2θ; Cu Kα1 radiation).

In one embodiment, the R-ketamine free base Form A is a crystalline polymorphic form characterized by PXRD peaks at 14.8 °2θ, 20.7 °2θ, and 23.8 °2θ (±0.2 °2θ; ±0.1 °2θ; or ±0.0 °2θ; Cu Kα1 radiation).

In one embodiment, the R-ketamine free base Form A is a crystalline polymorphic form characterized by a PXRD spectrum substantially similar to that shown in FIG. 1.

In one embodiment, the R-ketamine free base Form A is a crystalline polymorphic form characterized by one, two, three, four, five, six, seven, eight, nine, ten, eleven, twelve, thirteen, fourteen, fifteen or sixteen PXRD peaks set forth in Table 1.

In one embodiment, the R-ketamine free base Form A is characterized by FT-Raman spectrum substantially similar to that shown in FIG. 3.

In one embodiment, the R-ketamine free base Form A is characterized by a ¹H-NMR spectrum substantially similar to that shown in FIG. 6.

In one aspect, the present disclosure is directed to a pharmaceutical composition comprising one or more of the salts or salt forms described herein and a pharmaceutically acceptable excipient, diluent or carrier.

In one aspect, the present disclosure is directed to a pharmaceutical composition comprising one or more of the salts or salt forms described herein for use in the treatment or prevention of depression or depressive symptoms in a patient in need thereof.

In one aspect, the present disclosure is directed to a pharmaceutical composition comprising one or more of the salts or salt forms described herein for use in the treatment or prevention of treatment-resistant depression in a patient in need thereof.

In one aspect, the present disclosure is directed to the use of the salts or salt forms described herein in the preparation of a medicament for the treatment or prevention of depression or depressive symptoms in a patient in need thereof.

In one aspect, the present disclosure is directed to the use of the salts or salt forms described herein in the preparation of a medicament for the treatment or prevention of treatment-resistant depression or depressive symptoms in a patient in need thereof.

In one aspect, the present disclosure is directed to a method of treating or preventing depression or depressive symptoms comprising administering a salt, salt form, or pharmaceutical composition described herein to a patient in need thereof.

In one aspect, the present disclosure is directed to a method of treating or preventing treatment-resistant depression or depressive symptoms comprising administering a salt, salt form, or pharmaceutical composition described herein to a patient in need thereof.

Additional features, advantages, and aspects of the present disclosure are set forth or apparent from consideration of the following detailed description, drawings, and claims. Moreover, it is to be understood that both the foregoing summary of the present disclosure and the following detailed description are exemplary and intended to provide further explanation without limiting the scope of the present disclosure as claimed.

### BRIEF DESCRIPTION OF THE DRAWINGS

The accompanying drawings, which are included to provide a further understanding of the present disclosure, are incorporated in and constitute a part of this specification, illustrate aspects of the present disclosure and, together with the detailed description, serve to explain the principles of the present disclosure.

FIG. 1 depicts the PXRD pattern of the R-ketamine free base.

FIG. 2 depicts the PXRD pattern of the R-ketamine free base (top), and its hydrochloride salt starting material (bottom).

FIG. 3 depicts the overview of FT-Raman spectrum of the R-ketamine free base from 200 to 3500 cm⁻¹.

FIG. 4 depicts the fingerprint region of FT-Raman spectrum of the R-ketamine free base from 200 to 1800 cm⁻¹.

FIG. 5 depicts the TG-FTIR thermogram of the R-ketamine free base, shown with residual trace of dichloromethane, 0.57%; decomposition observed above 190° C.

FIG. 6 depicts the ¹H-NMR spectrum of the R-ketamine free base, recorded in DMSO-d6. The spectrum is consistent with the chemical structure of ketamine. Residual dichloromethane (<0.01 eq) is visible at 5.8 ppm.

FIG. 7A depicts the layout of the 96 well quartz plate showing the location of each salt former.

FIG. 7B depicts the solvents used for the evaporative experiments.

FIG. 8A depicts the layout of the 96 well quartz plate showing the location of each salt former and the solvent domains.

FIG. 8B depicts the solvents used for the slurry experiments.

FIG. 9 depicts the PXRD pattern of the R-ketamine saccharin salt.

FIG. 10 depicts the overlay of the PXRD patterns of saccharin (bottom), the R-ketamine saccharin salt (second from bottom), the R-ketamine free base (third from bottom), and R-ketamine HCl salt (top).

FIG. 11 depicts an overlay of the PXRD patterns of the wet R-ketamine saccharin salt (bottom), and the dried R-ketamine saccharin salt (top).

FIG. 12 depicts an overview of FT-Raman spectrum of the R-ketamine saccharin salt.

FIG. 13 depicts the fingerprint region of FT-Raman spectrum of the R-ketamine saccharin salt.

FIG. 14 depicts an overlay of the FT-Raman spectra of the R-ketamine saccharin salt from Example 3 (top), and the R-ketamine saccharin salt from Example 4 (bottom). Both samples present the same Raman spectrum.

FIG. 15 depicts the TG-FTIR of the R-ketamine saccharin salt.

FIG. 16 depicts the DSC of the R-ketamine saccharin salt.

FIG. 17 depicts the ¹H-NMR of the R-ketamine saccharin salt. The spectrum is consistent with a one to one saccharin salt, with water and residual isopropanol (0.009 eq).

FIG. 18 depicts the DVS isotherm of the R-ketamine saccharin salt: the change of water content (bottom) and relative humidity (top) as a function of time.

FIG. 19 depicts the DVS isotherm of the R-ketamine saccharin salt—the change of water content as a function of relative humidity.

FIG. 20 depicts an overlay of the PXRD patterns of the R-ketamine saccharin salt after DVS (bottom), and the R-ketamine saccharin salt before DVS (top).

FIG. 21 depicts the PXRD pattern of the R-ketamine fumarate salt Form A.

FIG. 22 depicts overlay of the PXRD patterns of fumaric acid (bottom), and the R-ketamine fumarate salt Form A (top).

FIG. 23 depicts an overlay of the PXRD patterns of the R-ketamine fumarate salt Form A (bottom), the R-ketamine free base (second from bottom), the R-ketamine HCl salt (second from top), and the R-ketamine saccharin salt (top).

FIG. 24 depicts the ¹H-NMR of the R-ketamine fumarate salt Form A. The spectrum is consistent with a 2:1 free base to fumaric acid salt.

FIG. 25 depicts the overview of FT-Raman spectrum of the R-ketamine fumarate salt.

FIG. 26 depicts the fingerprint region of FT-Raman spectrum of the R-ketamine fumarate salt.

FIG. 27 depicts an overlay of the FT-Raman spectra of the R-ketamine fumarate salt from Example 2 (arrows pointing to this trace), and the R-ketamine fumarate salt Form A of R-ketamine. The samples present different Raman spectra.

FIG. 28A depicts the TG-FTIR thermogram of the R-ketamine fumarate salt Form A.

FIG. 28B depicts the DSC thermogram of the R-ketamine hemifumarate salt Form A.

FIG. 28C depicts the DVS isotherm of the Form A: the change of water content (top) and relative humidity (bottom) as a function of time.

FIG. 28D depicts the DVS isotherm of the R-ketamine fumarate salt Form A: the change of water content as a function of relative humidity.

FIG. 28E depicts an overlay of the PXRD patterns of the R-ketamine fumarate salt Form A after DVS (bottom), and the R-ketamine fumarate salt Form A before DVS (top).

FIG. 29 depicts the PXRD pattern of the R-ketamine succinate salt.

FIG. 30 depicts an overlay of the PXRD patterns of the R-ketamine succinate salt (bottom), the R-ketamine free base (second from bottom), the R-ketamine HCl salt (middle), the R-ketamine saccharin salt (second from top), and the R-ketamine fumarate salt (top).

FIG. 31 depicts the ¹H-NMR of the R-ketamine succinate salt. The spectrum is consistent with a 1:1 free base to succinate salt.

FIG. 32 depicts the overview of FT-Raman spectrum of the R-ketamine succinate salt.

FIG. 33 depicts the fingerprint region of FT-Raman spectrum of the R-ketamine succinate salt.

FIG. 34 depicts an overlay of the FT-Raman spectra of the salt screen with succinic acid (top, with arrows), and the R-ketamine succinate salt (bottom).

FIG. 35 depicts the TG-FTIR thermogram of the R-ketamine succinate salt.

FIG. 36 depicts the PXRD pattern of the R-ketamine sulfate salt.

FIG. 37 depicts an overlay of the PXRD pattern of the R-ketamine sulfate salt (top) and the free base (bottom).

FIG. 38 depicts the ¹H-NMR of the R-ketamine sulfate salt. The spectrum is consistent with the R-ketamine sulfate structure with some residual TBME.

FIG. 39 depicts a comparison of the ¹H-NMR spectra of the R-ketamine sulfate salt (bottom) and the R-ketamine free base starting material (top).

FIG. 40 depicts the PXRD pattern of the R-ketamine D-tartrate salt Form A.

FIG. 41 depicts an overlay of the PXRD pattern of D-tartaric acid (bottom), the R-ketamine D-tartrate salt Form A (middle) and the free base (top).

FIG. 42 depicts the ¹H-NMR of the R-ketamine D-tartrate salt Form A. The spectrum is consistent with structure and approximately 0.8 eq of D-tartaric acid is observed.

FIG. 43 depicts the PXRD pattern of the R-ketamine oxalate salt.

FIG. 44 depicts an overlay of the PXRD pattern of oxalic acid (bottom), the R-ketamine oxalate salt (middle) and the free base (top).

FIG. 45A depicts the ¹H-NMR of the R-ketamine oxalate salt. The spectrum is consistent with the R-ketamine structure.

FIG. 45B depicts the TG-FTIR thermogram of the R-ketamine oxalate salt.

FIG. 45C depicts the DSC curve of the R-ketamine oxalate salt.

FIG. 45D depicts the overview of FT-Raman spectrum of the R-ketamine oxalate salt.

FIG. 45E depicts the fingerprint region of FT-Raman spectrum of the R-ketamine oxalate salt.

FIG. 45F depicts the DVS isotherm of the R-ketamine oxalate salt: the change of water content (top) and relative humidity (bottom) as a function of time.

FIG. 45G depicts the DVS isotherm of the R-ketamine oxalate salt: the change of water content as a function of relative humidity.

FIG. 45H depicts the overlay of the PXRD patterns of the R-ketamine oxalate salt after DVS (bottom), and the R-ketamine oxalate salt before DVS (top) FIG. 46 depicts the PXRD pattern of the R-ketamine citrate salt.

FIG. 47 depicts an overlay of the PXRD pattern of citric acid (bottom), the R-ketamine citrate salt (middle) and the free base (top).

FIG. 48 depicts the ¹H-NMR of the R-ketamine citrate salt with residual TBME and 2-propanol.

FIG. 49 depicts the overlay of the free base reference (blue trace), and location b in the well A7 of (arrows depict different R-ketamine free base crystalline form) with THF.

FIG. 50A depicts a representative crystal image of ADI R-ketamine salt.

FIG. 50B depicts a representative crystal image of ADI R-ketamine salt.

FIG. 50C depicts a representative crystal image of BZN R-ketamine salt.

FIG. 50D depicts a representative crystal image of BZN R-ketamine salt.

FIG. 50E depicts a representative crystal image of FUM R-ketamine salt.

FIG. 50F depicts a representative crystal image of SAC R-ketamine salt.

FIG. 50G depicts a representative crystal image of SUC R-ketamine salt.

FIG. 51A depicts a representative crystal image of BZN R-ketamine salt.

FIG. 51B depicts a representative crystal image of BZN R-ketamine salt.

FIG. 51C depicts a representative crystal image of FUM R-ketamine salt.

FIG. 51D depicts a representative crystal image of FUM R-ketamine salt.

FIG. 51E depicts a representative crystal image of SAC R-ketamine salt.

FIG. 51F depicts a representative crystal image of SUC R-ketamine salt.

FIG. 52 depicts an overlay of the PXRD pattern of the R-ketamine citrate salt (top) and the R-ketamine free base (scaled down 0.5 for better comparison, bottom). No peaks from citric acid are present.

FIG. 53 depicts the DSC curve of the R-ketamine D-tartrate salt Form A.

FIG. 54 depicts the overview of FT-Raman spectrum of the R-ketamine D-tartrate salt Form A.

FIG. 55 depicts the fingerprint region of FT-Raman spectrum of the R-ketamine D-tartrate salt Form A.

FIG. 56 depicts the PXRD pattern of the R-ketamine D-tartrate salt Form B.

FIG. 57 depicts an overlay of the PXRD patterns of the R-ketamine D-tartrate salt Form B (top) and the R-ketamine free base (bottom).

FIG. 58 depicts the ¹H-NMR of the R-ketamine D-tartrate salt Form B. The spectrum is consistent with a 1:1 R-ketamine to D-tartrate. Residual acetone (0.01 eq) is observed at 2.09 ppm FIG. 59 depicts an overview of FT-Raman spectrum of the R-ketamine D-tartrate salt Form B.

FIG. 60 depicts the fingerprint region of FT-Raman spectrum of the R-ketamine D-tartrate salt Form B.

FIG. 61 depicts the PXRD pattern of the R-ketamine D-tartrate salt Form C.

FIG. 62 depicts an overlay of the PXRD patterns of the R-ketamine D-tartrate salt Form A (top), the R-ketamine D-tartrate salt Form B (middle), and the R-ketamine D-tartrate salt Form C (bottom).

FIG. 63 depicts an overlay of the PXRD patterns of the R-ketamine D-tartrate salt Form C (top) and R-ketamine free base (bottom).

FIG. 64 depicts the ¹H-NMR of the R-ketamine D-tartrate salt Form C. The spectrum is consistent with a 1:1.5 R-ketamine to D-tartrate salt.

FIG. 65 depicts an overview of FT-Raman spectrum of the R-ketamine D-tartrate salt Form C.

FIG. 66 depicts fingerprint region of FT-Raman spectrum of the R-ketamine D-tartrate salt Form C.

FIG. 67 depicts an overlay of the Raman spectra of the R-ketamine D-tartrate salt Form A (blue trace), the R-ketamine D-tartrate salt Form B (red trace), and the R-ketamine D-tartrate salt Form C (green trace).

FIG. 68 depicts the PXRD pattern of the R-ketamine fumarate salt Form B.

FIG. 69 depicts an overlay of the PXRD patterns of fumaric acid (bottom), and the R-ketamine fumarate salt Form B (top).

FIG. 70 depicts an overlay of the PXRD patterns of the R-ketamine fumarate salt Form B (bottom), the R-ketamine fumarate salt Form A (middle), and the free base (top). The arrows show the reflections of the R-ketamine fumarate salt Form A that can be found in the R-ketamine fumarate salt Form B.

FIG. 71 depicts ¹H-NMR of the R-ketamine fumarate salt Form B. The spectrum is consistent with a 2:1 free base to fumaric acid salt. Residual THF is observed at 3.60 ppm.

FIG. 72 depicts the overview of FT-Raman spectrum of the R-ketamine fumarate salt Form B.

FIG. 73 depicts the fingerprint region of FT-Raman spectrum of the R-ketamine fumarate salt Form B.

FIG. 74 depicts an overlay the PXRD patterns of the R-ketamine free base (top), the R-ketamine D-tartrate salt Form A (middle), the R-ketamine D-tartrate salt Form B (bottom).

FIG. 75 depicts the DSC thermogram of the R-ketamine free base.

FIG. 76 depicts the TG-FTIR thermogram of the R-ketamine D-tartrate salt Form A.

FIG. 77 depicts the DVS isotherm of the R-ketamine D-tartrate salt Form A: the change of water content (top) and relative humidity (bottom) as a function of time.

FIG. 78 depicts the DVS isotherm of the R-ketamine D-tartrate salt Form A: the change of water content as a function of relative humidity.

FIG. 79 depicts the TG-FTIR thermogram of the R-ketamine D-tartrate salt Form B.

FIG. 80 depicts the DSC curve of the R-ketamine D-tartrate salt Form B.

FIG. 81 depicts the DVS isotherm of the R-ketamine D-tartrate salt Form B: the change of water content (top) and relative humidity (bottom) as a function of time.

FIG. 82 depicts the DVS isotherm of the R-ketamine D-tartrate salt Form B: the change of water content as a function of relative humidity.

FIG. 83 depicts the TG-FTIR thermogram of the R-ketamine D-tartrate salt Form C.

FIG. 84 depicts the DSC curve of the R-ketamine D-tartrate salt Form C.

FIG. 85 depicts the DVS isotherm of the R-ketamine D-tartrate salt Form C: the change of water content (top) and relative humidity (bottom) as a function of time.

FIG. 86 depicts the DVS isotherm of the R-ketamine D-tartrate salt Form C: the change of water content as a function of relative humidity.

FIG. 87 depicts the PXRD pattern of the R-ketamine D-tartrate salt Form D.

FIG. 88 depicts an overview of FT-Raman spectrum of the R-ketamine D-tartrate salt Form D.

FIG. 89 depicts fingerprint region of FT-Raman spectrum of the R-ketamine D-tartrate salt Form D.

FIG. 90 depicts the TG-FTIR thermogram of the R-ketamine D-tartrate salt Form D.

FIG. 91 depicts the DSC curve of the R-ketamine D-tartrate salt Form D.

FIG. 92 depicts the DVS isotherm of the R-ketamine D-tartrate salt Form D: the change of water content (top) and relative humidity (bottom) as a function of time.

FIG. 93 depicts the DVS isotherm of the R-ketamine D-tartrate salt Form D: the change of water content as a function of relative humidity.

FIG. 94 depicts an overlay of the PXRD patterns of R-ketamine D-tartrate salt Form C from Example 13 (top), Example 27 (middle), and Example 30 (bottom).

FIG. 95 depicts the ¹H-NMR of the R-ketamine D-tartrate salt Form D, which contains roughly 1 to 1.3 equivalents of tartaric acid.

FIG. 96 depicts an overlay of the PXRD patterns of the R-ketamine D-tartrate salt Form D (top), the R-ketamine D-tartrate salt Form C (second from top), the R-ketamine D-tartrate salt Form B (second from bottom), and the R-ketamine D-tartrate salt Form A (bottom).

FIG. 97 depicts an overlay of the PXRD patterns of the R-ketamine D-tartrate salt Form D (top) and a previously published PXRD pattern of S-ketamine L-tartrate dihydrate (bottom. calculated from SC-XRD data that was measured at −130° C. Taken from E. Ratti-Moberg, P. Groth, A. J. Aasen, Acta Chem. Scand., 1991, 45, 108)

FIG. 98 depicts the PXRD pattern of the R-ketamine L-tartrate salt Form A.

FIG. 99 depicts the PXRD pattern of the R-ketamine L-tartrate salt Form B.

FIG. 100 depicts an overlay of the PXRD patterns of the R-ketamine L-tartrate salt Form B (top) and R-ketamine L-tartrate salt Form A (bottom).

FIG. 101 depicts ¹H-NMR of the R-ketamine L-tartrate salt Form A. 0.68 eq of tartaric acid is observed. Residual 2-propanol is observed at 1.03 ppm.

FIG. 102 depicts ¹H-NMR of the R-ketamine L-tartrate salt Form B. Spectrum is consistent with a 1:1 free base to L-tartaric acid salt.

### DETAILED DESCRIPTION

The present disclosure provides salts and polymorphic salt forms of R-ketamine that are useful in the preparation of a medicament and/or as pharmaceutical agents. In some embodiments, one or more of the salts and/or salt forms described herein can be formulated into a pharmaceutical composition.

### Definitions

The terms “powder X-ray diffraction pattern”, “PXRD pattern”, “X-ray powder diffraction pattern”, and “XRPD pattern” are used interchangeably and refer to the experimentally observed diffractogram or parameters derived therefrom. Powder X-ray diffraction patterns are typically characterized by peak position (abscissa) and peak intensities (ordinate). The term “peak intensities” refers to relative signal intensities within a given X-ray diffraction pattern. Factors which can affect the relative peak intensities are sample thickness and preferred orientation (i.e., the crystalline particles are not distributed randomly). The term “peak positions” as used herein refers to X-ray reflection positions as measured and observed in powder X-ray diffraction experiments. Peak positions are directly related to the dimensions of the unit cell. The peaks, identified by their respective peak positions, are extracted from the diffraction patterns for the various polymorphic forms of salts of R-ketamine.

The term “2 theta value”, “2θ” or “2 θ” refers to the peak position in degrees based on the experimental setup of the X-ray diffraction experiment and is a common abscissa unit in diffraction patterns. In general, the experimental setup requires that if a reflection is diffracted when the incoming beam forms an angle theta (θ) with a certain lattice plane, the reflected beam is recorded at an angle 2 theta (2 θ). It should be understood that reference herein to specific 2θ values for a specific polymorphic form is intended to mean the 2θ values (in degrees) as measured using the X-ray diffraction experimental conditions as described herein.

“Preferred orientation effects” refer to variable peak intensities or relative intensity differences between different PXRD measurements of the same samples that can be due to the orientation of the particles. Without wishing to be bound by theory, in PXRD it can be desirable to have a sample in which particles are oriented randomly (e.g., a powder). However, it can be difficult or in some cases impossible to achieve truly random particle orientations in practice. As particle size increases, the randomness of particle orientation can decrease, leading to increased challenges with achieving a preferred orientation. Without wishing to be bound by theory, a smaller particle size can reduce technical challenges associated with preferred orientation and allow for more accurate representation of peaks. However, one of skill in the art will understand how to reduce or mitigate preferred orientation effects and will recognize preferred orientation effects that can exist even between two different measurements of the same sample. For instance, in some embodiments, differences in resolution or relative peak intensities can be attributed to preferred orientation effects.

As used herein, the term “substantially pure” with reference to a particular salt (or to a mixture of two or more salts) of a compound indicates the salt (or a mixture) includes less than 10%, less than 5%, less than 3%, less than 1%, less than 0.5%, less than 0.2%, or less than 0.1% by weight of impurities, including other salt forms of the compound. Such purity may be determined, for example, by powder X-ray diffraction.

As used herein, the term “polymorph” or “salt form” refers to different crystalline forms of the same compound and other solid state molecular forms including pseudo-polymorphs, such as hydrates (e.g., bound water present in the crystalline structure) and solvates (e.g., bound solvents other than water) of the same compound. Different crystalline polymorphs have different crystal structures due to a different packing of the molecules in the lattice. This results in a different crystal symmetry and/or unit cell parameters which directly influences its physical properties such as the X-ray diffraction characteristics of crystals or powders. A different polymorph, for example, will in general diffract at a different set of angles and will give different values for the intensities. Therefore, X-ray powder diffraction can be used to identify different polymorphs, or a solid form that comprises more than one polymorph, in a reproducible and reliable way (S. Byrn et al, Pharmaceutical Solids: A Strategic Approach to Regulatory Considerations, Pharmaceutical research, Vol. 12, No. 7, p. 945-954, 1995; J. K. Haleblian and W. McCrone, Pharmaceutical Applications of Polymorphism, Journal of Pharmaceutical Sciences, Vol. 58, No. 8, p. 911-929, 1969).

Crystalline polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since impurities present may produce undesired toxicological effects. Certain polymorphic forms may exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies.

The term “amorphous” refers to any solid substance which (i) lacks order in three dimensions, or (ii) exhibits order in less than three dimensions, order only over short distances (e.g., less than 10 A), or both. Thus, amorphous substances include partially crystalline materials and crystalline mesophases with, e.g., one- or two-dimensional translational order (liquid crystals), orientational disorder (orientationally disordered crystals), or conformational disorder (conformationally disordered crystals). Amorphous solids may be characterized by known techniques, including powder X-ray diffraction (PXRD) crystallography, solid state nuclear magnet resonance (ssNMR) spectroscopy, differential scanning calorimetry (DSC), or some combination of these techniques. Amorphous solids give diffuse PXRD patterns, typically comprised of one or two broad peaks (i.e., peaks having base widths of about 5° 2Θ or greater).

The term “crystalline” refers to any solid substance exhibiting three-dimensional order, which in contrast to an amorphous solid substance, gives a distinctive PXRD pattern with sharply defined peaks.

The term “ambient temperature” refers to a temperature condition typically encountered in a laboratory setting. This includes the approximate temperature range of about 20 to about 30° C.

The term “detectable amount” refers to an amount or amount per unit volume that can be detected using conventional techniques, such as X-ray powder diffraction, differential scanning calorimetry, HPLC, Fourier Transform Infrared Spectroscopy (FT-IR), Raman spectroscopy, and the like.

The term “solvate” describes a molecular complex comprising the drug substance and a stoichiometric or non-stoichiometric amount of one or more solvent molecules (e.g., ethanol).

When the solvent is tightly bound to the drug the resulting complex will have a well-defined stoichiometry that is independent of humidity. When, however, the solvent is weakly bound, as in channel solvates and hygroscopic compounds, the solvent content will be dependent on humidity and drying conditions. In such cases, the complex may be non-stoichiometric.

The term “hydrate” describes a solvate comprising the drug substance and a stoichiometric or non-stoichiometric amount of water.

The term “relative humidity” refers to the ratio of the amount of water vapor in air at a given temperature to the maximum amount of water vapor that can be held at that temperature and pressure, expressed as a percentage.

The term “relative intensity” refers to an intensity value derived from a sample X-ray diffraction pattern. The complete ordinate range scale for a diffraction pattern is assigned a value of 100. A peak having intensity falling between about 50% to about 100% on this scale intensity is termed very strong (vs); a peak having intensity falling between about 50% to about 25% is termed strong (s). Additional weaker peaks are present in typical diffraction patterns and are also characteristic of a given polymorph, wherein the additional peaks are termed medium (m), weak (w) and very weak (vw).

The term “slurry” refers to a solid substance suspended in a liquid medium, typically water or an organic solvent.

The term “under vacuum” refers to typical pressures obtainable by a laboratory oil or oil-free diaphragm vacuum pump.

The term “pharmaceutical composition” refers to a composition comprising one or more of the polymorphic forms of salts of R-ketamine described herein, and other chemical components, such as physiologically/pharmaceutically acceptable carriers, diluents, vehicles and/or excipients. The purpose of a pharmaceutical composition is to facilitate administration of a compound to an organism, such as a human or other mammals.

The term “pharmaceutically acceptable” “carrier”, “diluent”, “vehicle”, or “excipient” refers to a material (or materials) that may be included with a particular pharmaceutical agent to form a pharmaceutical composition, and may be solid or liquid. Exemplary solid carriers are lactose, sucrose, talc, gelatin, agar, pectin, acacia, magnesium stearate, stearic acid and the like.

Exemplary liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropyl methylcellulose, methylmethacrylate and the like.

The term “treating”, as used herein, unless otherwise indicated, means reversing, alleviating, or inhibiting the progress of the disorder or condition to which such term applies, or one or more symptoms of such disorder or condition. The term “treatment”, as used herein, unless otherwise indicated, refers to the act of “treating” as defined immediately above. For example, the terms “treat”, “treating” and “treatment” can refer to a method of alleviating or abrogating a particular disorder and/or one or more of its attendant symptoms.

As used herein, “subject” means a human or animal (in the case of an animal, the subject can be a mammal). In one aspect, the subject is a human. In one aspect, the subject is a male. In one aspect, the subject is a female.

The term “about” is used herein to mean approximately, in the region of, roughly or around. When the term “about” is used in conjunction with a numerical range, it modifies that range by extending the boundaries above and below the numerical values set forth. In general, the term “about” is used herein to modify a numerical value above and below the stated value by a variance of 20%, a variance of 10%, a variance of 5%, a variance of 3%, or a variance of 1%. When used in the context of XRPD peak values, the term “about” can indicate a peak value ±0.20, ±0.15, ±0.10, ±0.05, or +0.01 °2θ. In some embodiments, when used in the context of XRPD peak values “about” can indicate a peak value at substantially exactly the disclosed peak value.

### R-Ketamine Crystalline Forms

As set forth below, R-ketamine can form salts with different acids. In some embodiments, the R-ketamine salts described herein exist in various crystalline forms. All PXRD peaks described herein are in °2θ (±0.2 °2θ; ±0.1 °2θ; or ±0.0 °2θ; Cu Kα1 radiation). Additionally, all PXRD spectra are obtained using Cu Kα1 X-rays at a wavelength of 1.5406 Å.

### R-ketamine Free Base

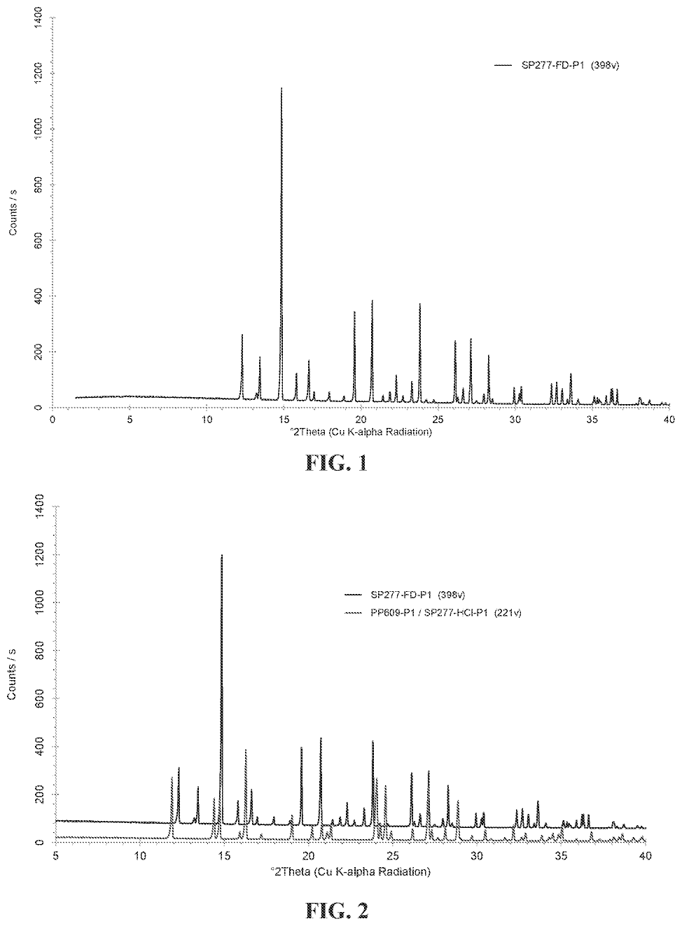

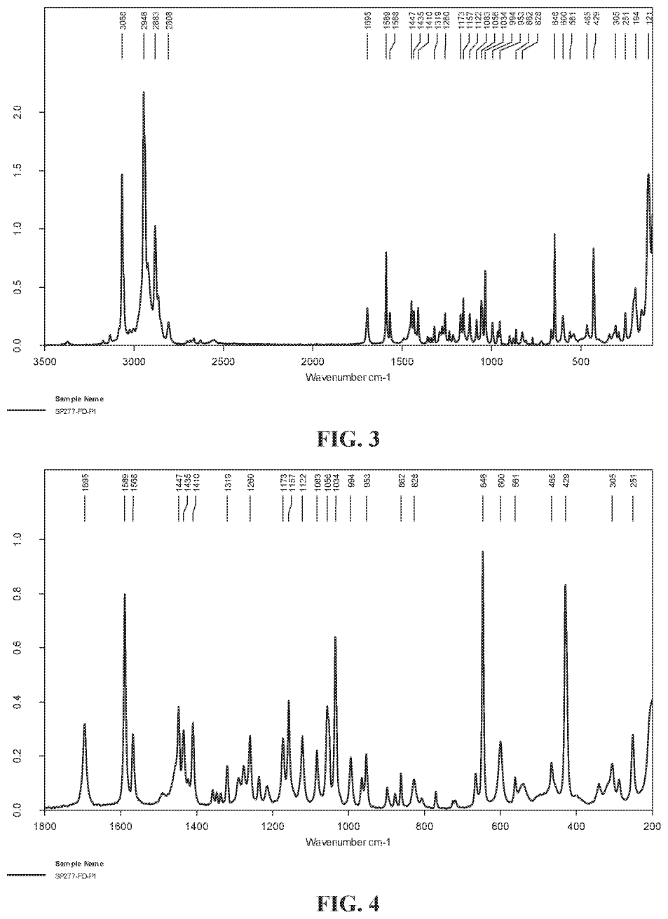

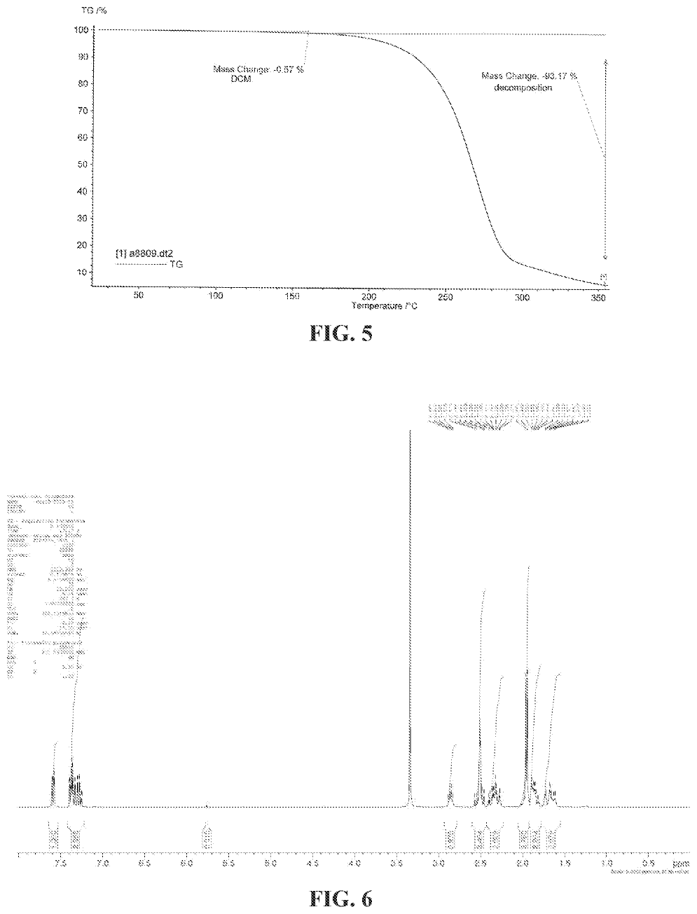

In some embodiments, the R-ketamine of the present disclosure is a free base. In some embodiments, the R-ketamine free base is crystalline. In some embodiments, the R-ketamine free base is a crystalline polymorphic Form A. In some embodiments, the R-ketamine free base Form A is characterized by the PXRD peaks set forth below in Table 1. In some embodiments, the R-ketamine free base PXRD spectrum is substantially similar to that shown in FIG. 1. In some embodiments, the R-ketamine free base FT-Raman spectrum is substantially similar to that shown in FIG. 3. In some embodiments, the R-ketamine free base ¹H-NMR spectrum is substantially similar to that shown in FIG. 6.

In some embodiments, the R-ketamine free base is a crystalline polymorphic Form A characterized by a PXRD peak at 14.8 °2θ (±0.2 °2θ; ±0.1 °2θ; or ±0.0 °2θ; Cu Kα1 radiation). In some embodiments, the R-ketamine free base is a crystalline polymorphic Form A characterized by PXRD peaks at 14.8 °2θ and 20.7 °2θ (±0.2 °2θ; ±0.1 °2θ; or ±0.0 °2θ; Cu Kα1 radiation). In some embodiments, the R-ketamine free base is a crystalline polymorphic Form A characterized by PXRD peaks at two or more, or three or more peaks selected from the group consisting of 14.8 °2θ, 20.7 °2θ, and 23.8 °2θ (±0.2 °2θ; ±0.1 °2θ; or ±0.0 °2θ; Cu Kα1 radiation). In some embodiments, the R-ketamine free base is a crystalline polymorphic Form A characterized by PXRD peaks at 14.8 °2θ, 20.7 °2θ, and 23.8 °2θ (±0.2 °2θ; ±0.1 °2θ; or ±0.0 °2θ; Cu Kα1 radiation).

In some embodiments, the R-ketamine free base is a crystalline polymorphic Form A characterized by PXRD peaks at two or more, or three or more peaks selected from the group consisting of 12.3 °2θ, 14.8 °2θ, 20.7 °2θ, and 23.8 °2θ (±0.2 °2θ; ±0.1 °2θ; or ±0.0 °2θ; Cu Kα1 radiation). In some embodiments, the R-ketamine free base is a crystalline polymorphic form characterized by PXRD peaks at 12.3 °2θ, 14.8 °2θ, 20.7 °2θ, and 23.8 °2θ, (±0.2 °2θ; ±0.1 °2θ; or +0.0 °2θ; Cu Kα1 radiation).

In some embodiments, the R-ketamine free base is a crystalline polymorphic Form A characterized by PXRD peaks at two or more, or three or more peaks selected from the group consisting of 14.8 °2θ, 19.6 °2θ, 20.7 °2θ, and 23.8 °2θ (±0.2 °2θ; ±0.1 °2θ; or ±0.0 °2θ; Cu Kα1 radiation). In some embodiments, the R-ketamine free base is a crystalline polymorphic Form A characterized by PXRD peaks at 14.8 °2θ, 19.6 °2θ, 20.7 °2θ, and 23.8 °2θ, (±0.2 °2θ; ±0.1 °2θ; or +0.0 °2θ; Cu Kα1 radiation).

In some embodiments, the R-ketamine free base is a crystalline polymorphic Form A characterized by PXRD peaks at two or more, or three or more peaks selected from the group consisting of 12.3 °2θ, 14.8 °2θ, 19.6 °2θ, 20.7 °2θ, and 23.8 °2θ (±0.2 °2θ; ±0.1 °2θ; or ±0.0 °2θ; Cu Kα1 radiation). In some embodiments, the R-ketamine free base is a crystalline polymorphic Form A characterized by PXRD peaks at 12.3 °2θ, 14.8 °2θ, 19.6 °2θ, 20.7 °2θ, and 23.8 °2θ (±0.2 °2θ; ±0.1 °2θ; or ±0.0 °2θ; Cu Kα1 radiation).

In some embodiments, the R-ketamine free base is a crystalline polymorphic Form A characterized by PXRD peaks at two or more, or three or more peaks selected from the group consisting of 12.3 °2θ, 14.8 °2θ, 19.6 °2θ, 20.7 °2θ, 23.8 °2θ, and 27.1 °2θ, (±0.2 °2θ; ±0.1 °2θ; or +0.0 °2θ; Cu Kα1 radiation). In some embodiments, the R-ketamine free base is a crystalline polymorphic Form A characterized by PXRD peaks at 12.3 °2θ, 14.8 °2θ, 19.6 °2θ, 20.7 °2θ, 23.8 °2θ, and 27.1 °2θ, (±0.2 °2θ; ±0.1 °2θ; or ±0.0 °2θ; Cu Kα1 radiation).

In some embodiments, the R-ketamine free base is a crystalline polymorphic Form A characterized by PXRD peaks at two or more, or three or more peaks selected from the group consisting of 12.3 °2θ, 14.8 °2θ, 19.6 °2θ, 20.7 °2θ, 23.8 °2θ, 26.1 °2θ, and 27.1 °2θ, (±0.2 °2θ; ±0.1 °2θ; or ±0.0 °2θ; Cu Kα1 radiation). In some embodiments, the R-ketamine free base is a crystalline polymorphic Form A characterized by PXRD peaks at 12.3 °2θ, 14.8 °2θ, 19.6 °2θ, 20.7 °2θ, 23.8 °2θ, 26.1 °2θ, and 27.1 °2θ, (±0.2 °2θ; ±0.1 °2θ; or ±0.0 °2θ; Cu Kα1 radiation).

In some embodiments, the R-ketamine free base is a crystalline polymorphic Form A characterized by PXRD peaks at two or more, or three or more peaks selected from the group consisting of 12.3 °2θ, 14.8 °2θ, 19.6 °2θ, 20.7 °2θ, 23.8 °2θ, 26.1 °2θ, 27.1 °2θ, and 28.3 °2θ, (±0.2 °2θ; ±0.1 °2θ; or ±0.0 °2θ; Cu Kα1 radiation). In some embodiments, the R-ketamine free base is a crystalline polymorphic Form A characterized by PXRD peaks at 12.3 °2θ, 14.8 °2θ, 19.6 °2θ, 20.7 °2θ, 23.8 °2θ, 26.1 °2θ, 27.1 °2θ, and 28.3 °2θ, (±0.2 °2θ; ±0.1 °2θ; or ±0.0 °2θ; Cu Kα1 radiation).

In some embodiments, the R-ketamine free base is a crystalline polymorphic Form A characterized by PXRD peaks at two or more, or three or more peaks selected from the group consisting of 12.3 °2θ, 13.4 °2θ, 14.8 °2θ, 19.6 °2θ, 20.7 °2θ, 23.8 °2θ, 26.1 °2θ, 27.1 °2θ, and 28.3 °2θ, (±0.2 °2θ; ±0.1 °2θ; or ±0.0 °2θ; Cu Kα1 radiation). In some embodiments, the R-ketamine free base is a crystalline polymorphic Form A characterized by PXRD peaks at 12.3 °2θ, 13.4 °2θ, 14.8 °2θ, 19.6 °2θ, 20.7 °2θ, 23.8 °2θ, 26.1 °2θ, 27.1 °2θ, and 28.3 °2θ, (±0.2 °2θ; ±0.1 °2θ; or ±0.0 °2θ; Cu Kα1 radiation).

In some embodiments, the R-ketamine free base is a crystalline polymorphic Form A characterized by PXRD peaks at two or more, or three or more peaks selected from the group consisting of 12.3 °2θ, 13.4 °2θ, 14.8 °2θ, 16.6 °2θ, 19.6 °2θ, 20.7 °2θ, 23.8 °2θ, 26.1 °2θ, 27.1 °2θ, and 28.3 °2θ, (±0.2 °2θ; ±0.1 °2θ; or ±0.0 °2θ; Cu Kα1 radiation). In some embodiments, the R-ketamine free base is a crystalline polymorphic Form A characterized by PXRD peaks at 12.3 °2θ, 13.4 °2θ, 14.8 °2θ, 16.6 °2θ, 19.6 °2θ, 20.7 °2θ, 23.8 °2θ, 26.1 °2θ, 27.1 °2θ, and 28.3 °2θ, (±0.2 °2θ; ±0.1 °2θ; or ±0.0 °2θ; Cu Kα1 radiation).

In some embodiments, the R-ketamine free base is a crystalline polymorphic Form A characterized by PXRD peaks at two or more, or three or more peaks selected from the group consisting of 12.3 °2θ, 13.4 °2θ, 14.8 °2θ, 15.8 °2θ, 16.6 °2θ, 19.6 °2θ, 20.7 °2θ, 23.8 °2θ, 26.1 °2θ, 27.1 °2θ, and 28.3 °2θ, (±0.2 °2θ; ±0.1 °2θ; or ±0.0 °2θ; Cu Kα1 radiation). In some embodiments, the R-ketamine free base is a crystalline polymorphic Form A characterized by PXRD peaks at 12.3 °2θ, 13.4 °2θ, 14.8 °2θ, 15.8 °2θ, 16.6 °2θ, 19.6 °2θ, 20.7 °2θ, 23.8 °2θ, 26.1 °2θ, 27.1 °2θ, and 28.3 °2θ, (±0.2 °2θ; ±0.1 °2θ; or ±0.0 °2θ; Cu Kα1 radiation).

In some embodiments, the R-ketamine free base is a crystalline polymorphic Form A characterized by PXRD peaks at two or more, or three or more peaks selected from the group consisting of 12.3 °2θ, 13.4 °2θ, 14.8 °2θ, 15.8 °2θ, 16.6 °2θ, 19.6 °2θ, 20.7 °2θ, 23.8 °2θ, 26.1 °2θ, 27.1 °2θ, 28.3 °2θ, and 33.6 °2θ (±0.2 °2θ; ±0.1 °2θ; or ±0.0 °2θ; Cu Kα1 radiation). In some embodiments, the R-ketamine free base is a crystalline polymorphic Form A characterized by PXRD peaks at 12.3 °2θ, 13.4 °2θ, 14.8 °2θ, 15.8 °2θ, 16.6 °2θ, 19.6 °2θ, 20.7 °2θ, 23.8 °2θ, 26.1 °2θ, 27.1 °2θ, 28.3 °2θ, and 33.6 °2θ (±0.2 °2θ; ±0.1 °2θ; or ±0.0 °2θ; Cu Kα1 radiation).

In some embodiments, the R-ketamine free base is a crystalline polymorphic Form A characterized by PXRD peaks at two or more, or three or more peaks selected from the group consisting of 12.3 °2θ, 13.4 °2θ, 14.8 °2θ, 15.8 °2θ, 16.6 °2θ, 19.6 °2θ, 20.7 °2θ, 22.3 °2θ, 23.8 °2θ, 26.1 °2θ, 27.1 °2θ, 28.3 °2θ, and 33.6 °2θ (±0.2 °2θ; ±0.1 °2θ; or ±0.0 °2θ; Cu Kα1 radiation). In some embodiments, the R-ketamine free base is a crystalline polymorphic Form A characterized by PXRD peaks at 12.3 °2θ, 13.4 °2θ, 14.8 °2θ, 15.8 °2θ, 16.6 °2θ, 19.6 °2θ, 20.7 °2θ, 22.3 °2θ, 23.8 °2θ, 26.1 °2θ, 27.1 °2θ, 28.3 °2θ, and 33.6 °2θ (±0.2 °2θ; ±0.1 °2θ; or ±0.0 °2θ; Cu Kα1 radiation).

In some embodiments, the R-ketamine free base Form A is characterized by one, two, three, four, five, six, seven, eight, nine, ten, eleven, twelve, thirteen, fourteen, fifteen or sixteen PXRD peaks selected from those set forth in Table 1.

TABLE 1
R-ketamine Free Base Form A PXRD (±0.2 °2θ; ±0.1 °2θ;
or ±0.0 °2θ; Cu Kα1 radiation).

| Angle in | d-spacing | | |
|---|---|---|---|
| °2-theta | in Å | Intensity | Intensity % |
| 12.3 | 7.21 | m | 23 |
| 13.2 | 6.72 | vw | 4 |
| 13.4 | 6.59 | m | 16 |
| 14.8 | 5.97 | vs | 100 |
| 15.8 | 5.61 | w | 11 |
| 16.6 | 5.33 | m | 15 |
| 17.0 | 5.22 | w | 5 |
| 17.9 | 4.94 | vw | 5 |
| 18.9 | 4.69 | vw | 4 |
| 19.6 | 4.53 | s | 31 |
| 20.7 | 4.29 | s | 34 |
| 21.4 | 4.14 | vw | 4 |
| 21.9 | 4.06 | vw | 5 |
| 22.3 | 3.98 | w | 10 |
| 22.7 | 3.91 | vw | 4 |
| 23.3 | 3.81 | w | 8 |
| 23.8 | 3.73 | s | 33 |
| 24.2 | 3.67 | vw | 3 |
| 24.7 | 3.60 | vw | 3 |
| 26.1 | 3.41 | m | 21 |
| 26.6 | 3.35 | w | 6 |
| 27.1 | 3.28 | m | 22 |
| 27.5 | 3.24 | vw | 2 |
| 28.0 | 3.19 | vw | 4 |
| 28.3 | 3.15 | m | 17 |
| 28.6 | 3.12 | vw | 2 |
| 29.9 | 2.98 | w | 6 |
| 30.4 | 2.94 | vw | 4 |
| 32.4 | 2.76 | w | 8 |
| 32.7 | 2.74 | w | 8 |
| 33.0 | 2.71 | w | 6 |
| 33.3 | 2.69 | vw | 2 |
| 33.6 | 2.66 | w | 11 |
| 34.1 | 2.63 | vw | 3 |
| 35.1 | 2.55 | vw | 4 |
| 35.4 | 2.53 | vw | 2 |
| 35.9 | 2.50 | vw | 4 |
| 36.3 | 2.47 | vw | 4 |
| 36.6 | 2.45 | w | 6 |
| 37.8 | 2.38 | vw | 1 |
| 38.1 | 2.36 | vw | 3 |
| 38.7 | 2.32 | vw | 2 |
| 39.5 | 2.28 | vw | 2 |
| 39.8 | 2.26 | vw | 2 |
| 40.1 | 2.25 | vw | 3 |
| 40.4 | 2.23 | vw | 1 |

In some embodiments, the R-ketamine free base is a polymorphic Form B. In some embodiments, the R-ketamine free base is a polymorphic Form B formed with THE. In some embodiments, the R-ketamine free base is a THF-solvate. In some embodiments, the R-ketamine free base is a polymorphic Form B solvate. In some embodiments, the R-ketamine free base is a polymorphic Form B THE solvate.

### R-ketamine Salts and Crystal Forms Thereof

### R-Ketamine Saccharin Salt

In one aspect, the present disclosure provides an R-ketamine saccharin salt. In some embodiments, the R-ketamine saccharin salt is amorphous. In some embodiments, the R-ketamine saccharin salt is crystalline. In some embodiments, the R-ketamine saccharin salt is a crystalline polymorphic form. In some embodiments, the R-ketamine saccharin salt is a crystalline polymorphic form characterized by the PXRD peaks set forth below in Table 2.

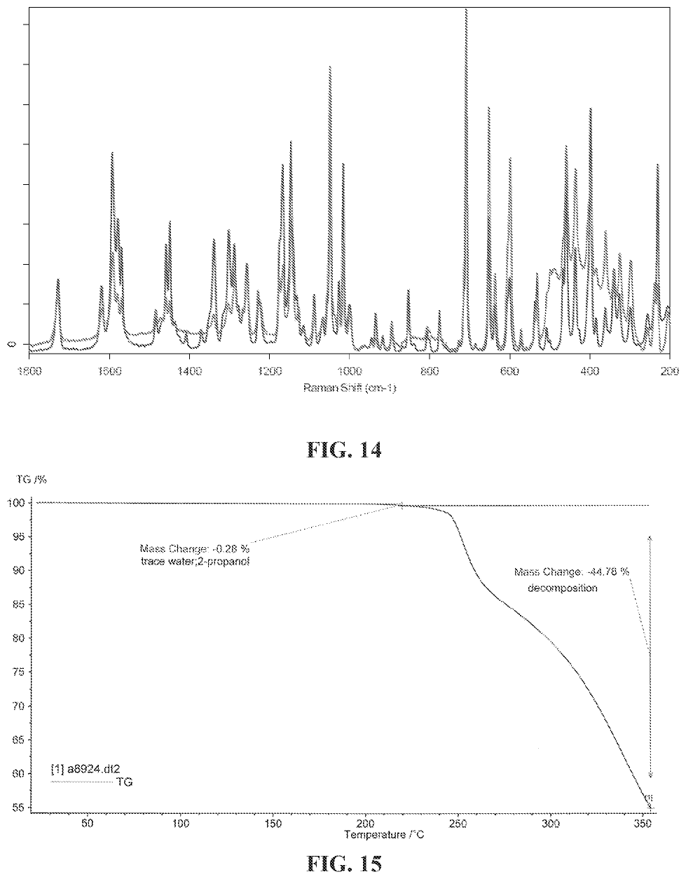

In some embodiments, the R-ketamine saccharin salt is a crystalline polymorphic form characterized by PXRD spectrum substantially similar to that shown in FIG. 9. In some embodiments, the R-ketamine saccharin salt is characterized by FT-Raman spectrum substantially similar to that shown in FIG. 14. In some embodiments, the R-ketamine saccharin salt is characterized by TG-FTIR substantially similar to that shown in FIG. 15. In some embodiments, the R-ketamine saccharin salt is a 2-propanol solvate. In some embodiments, the R-ketamine saccharin salt is a hydrate. In some embodiments, the R-ketamine saccharin salt is a 2-propanol:water solvate. In some embodiments, the R-ketamine saccharin salt is anhydrous.

In some embodiments, the R-ketamine saccharin salt is characterized by DSC having a melting peak at 210.5° C. In some embodiments, the R-ketamine saccharin salt is characterized by DSC having a melting peak onset at 209.2° C. In some embodiments, the R-ketamine saccharin salt is characterized by DSC having an associated enthalpy of 139.3 J/g.

In some embodiments, the R-ketamine saccharin salt is characterized by ¹H-NMR substantially similar to FIG. 17.

In some embodiments, the R-ketamine saccharin salt is a 1:1 R-ketamine:saccharin salt. In some embodiments, the R-ketamine saccharin salt is a 2:1 R-ketamine:saccharin salt. In some embodiments, the R-ketamine saccharin salt is a 1:2 R-ketamine:saccharin salt.

In some embodiments, the R-ketamine saccharin salt is a crystalline polymorphic form characterized by a PXRD peak at 13.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 13.8 °2θ and 23.5 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.6 °2θ, and 23.5 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 13.8 °2θ, 15.6 °2θ, and 23.5 °2θ (10.2 °2θ; 10.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.6 °2θ, 20.5 °2θ, and 23.5 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 13.8 °2θ, 15.6 °2θ, 20.5 °2θ, and 23.5 °2θ (±0.2 °2θ; ±0.1 °2θ; or +0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.6 °2θ, 16.0 °2θ, 20.5 °2θ, and 23.5 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 13.8 °2θ, 15.6 °2θ, 16.0 °2θ, 20.5 °2θ, and 23.5 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.6 °2θ, 16.0 °2θ, 20.5 °2θ, 22.0 °2θ, and 23.5 °2θ (±0.2 °2θ; ±0.1 °2θ; or +0.0 °2θ; Cu Kα1 radiation).

In some embodiments, the R-ketamine saccharin salt is a crystalline polymorphic form characterized by PXRD peaks at 13.8 °2θ, 15.6 °2θ, 16.0 °2θ, 20.5 °2θ, 22.0 °2θ, and 23.5 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.6 °2θ, 16.0 °2θ, 20.2 °2θ, 20.5 °2θ, 22.0 °2θ, and 23.5 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 13.8 °2θ, 15.6 °2θ, 16.0 °2θ, 20.2 °2θ, 20.5 °2θ, 22.0 °2θ, and 23.5 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.6 °2θ, 16.0 °2θ, 20.2 °2θ, 20.5 °2θ, 22.0 °2θ, 23.5 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 13.8 °2θ, 15.6 °2θ, 16.0 °2θ, 20.2 °2θ, 20.5 °2θ, 22.0 °2θ, 23.5 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.6 °2θ, 16.0 °2θ, 20.0 °2θ, 20.2 °2θ, 20.6 °2θ, 22.0 °2θ, 23.5 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 13.8 °2θ, 15.6 °2θ, 16.0 °2θ, 20.0 °2θ, 20.2 °2θ, 20.5 °2θ, 22.0 °2θ, 23.5 °2θ, and 26.8 °2θ (±0.2 °2θ; ±0.1 °2θ; or 0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.3 °2θ, 15.6 °2θ, 16.0 °2θ, 20.0 °2θ, 20.2 °2θ, 20.5 °2θ, 22.0 °2θ, 23.5 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 13.8 °2θ, 15.3 °2θ, 15.6 °2θ, 16.0 °2θ, 20.0 °2θ, 20.2 °2θ, 20.5 °2θ, 22.0 °2θ, 23.5 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.3 °2θ, 15.6 °2θ, 16.0 °2θ, 20.0 °2θ, 20.2 °2θ, 20.5 °2θ, 22.0 °2θ, 23.5 °2θ, 25.2 °2θ, and 26.8 °2θ (±0.2 °2θ; ±0.1 °2θ; or 0.0 °2θ; Cu Kα1 radiation).

In some embodiments, the R-ketamine saccharin salt is a crystalline polymorphic form characterized by PXRD peaks at 13.8 °2θ, 15.3 °2θ, 15.6 °2θ, 16.0 °2θ, 20.0 °2θ, 20.2 °2θ, 20.5 °2θ, 22.0 °2θ, 23.5 °2θ, 25.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.3 °2θ, 15.6 °2θ, 16.0 °2θ, 20.0 °2θ, 20.2 °2θ, 20.5 °2θ, 22.021.96 °2θ, 22.2 °2θ, 23.5 °2θ, 25.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 13.8 °2θ, 15.3 °2θ, 15.6 °2θ, 16.0 °2θ, 20.0 °2θ, 20.2 °2θ, 20.5 °2θ, 22.0 °2θ, 22.2 °2θ, 23.5 °2θ, 25.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.3 °2θ, 15.6 °2θ, 16.0 °2θ, 19.4 °2θ, 20.0 °2θ, 20.2 °2θ, 20.5 °2θ, 22.0 °2θ, 22.2 °2θ, 23.5 °2θ, 25.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 13.8 °2θ, 15.3 °2θ, 15.6 °2θ, 16.0 °2θ, 19.4 °2θ, 20.0 °2θ, 20.2 °2θ, 20.5 °2θ, 22.0 °2θ, 22.2 °2θ, 23.5 °2θ, 25.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 13.8 °2θ, 15.3 °2θ, 15.6 °2θ, 16.0 °2θ, 19.4 °2θ, 20.0 °2θ, 20.2 °2θ, 20.5 °2θ, 22.0 °2θ, 22.2 °2θ, 23.5 °2θ, 25.2 °2θ, 26.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 13.8 °2θ, 15.3 °2θ, 15.6 °2θ, 16.0 °2θ, 19.4 °2θ, 20.0 °2θ, 20.2 °2θ, 20.5 °2θ, 22.0 °2θ, 22.2 °2θ, 23.5 °2θ, 25.2 °2θ, 26.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 7.8 °2θ, 13.8 °2θ, 15.3 °2θ, 15.6 °2θ, 16.0 °2θ, 19.4 °2θ, 20.0 °2θ, 20.2 °2θ, 20.5 °2θ, 22.0 °2θ, 22.2 °2θ, 23.5 °2θ, 25.2 °2θ, 26.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 7.8 °2θ, 13.8 °2θ, 15.3 °2θ, 15.6 °2θ, 16.0 °2θ, 19.4 °2θ, 20.0 °2θ, 20.2 °2θ, 20.5 °2θ, 22.0 °2θ, 22.2 °2θ, 23.5 °2θ, 25.2 °2θ, 26.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 7.8 °2θ, 13.8 °2θ, 15.3 °2θ, 15.6 °2θ, 16.0 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.2 °2θ, and 26.8 °2θ (±0.2 °2θ; ±0.1 °2θ; or +0.0 °2θ; Cu Kα1 radiation).

In some embodiments, the R-ketamine saccharin salt is a crystalline polymorphic form characterized by PXRD peaks at 7.8 °2θ, 13.8 °2θ, 15.3 °2θ, 15.6 °2θ, 16.0 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 7.8 °2θ, 13.8 °2θ, 15.3 °2θ, 15.6 °2θ, 16.0 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.0 °2θ, 26.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 7.8 °2θ, 13.8 °2θ, 15.3 °2θ, 15.6 °2θ, 16.0 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.0 °2θ, 26.2 °2θ, and 26.8 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 7.8 °2θ, 13.8 °2θ, 15.3 °2θ, 15.6 °2θ, 16.0 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.0 °2θ, 26.2 °2θ, 26.8 °2θ, and 28.7 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 7.8 °2θ, 13.8 °2θ, 15.3 °2θ, 15.6 °2θ, 16.0 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.0 °2θ, 26.2 °2θ, 26.8 °2θ, and 28.7 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 7.8 °2θ, 13.8 °2θ, 15.3 °2θ, 15.6 °2θ, 16.0 °2θ, 16.6 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.0 °2θ, 26.2 °2θ, 26.8 °2θ, and 28.7 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 7.8 °2θ, 13.8 °2θ, 15.3 °2θ, 15.6 °2θ, 16.0 °2θ, 16.6 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.0 °2θ, 26.2 °2θ, 26.8 °2θ, and 28.7 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 7.8 °2θ, 13.8 °2θ, 15.3 °2θ, 15.6 °2θ, 16.0 °2θ, 16.6 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.0 °2θ, 26.2 °2θ, 26.8 °2θ, 28.7 °2θ, and 32.9 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at 7.8 °2θ, 13.8 °2θ, 15.3 °2θ, 15.6 °2θ, 16.0 °2θ, 16.6 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.0 °2θ, 26.2 °2θ, 26.8 °2θ, 28.7 °2θ, and 32.9 °2θ (±0.2 °2θ; ±0.1 °2θ; or ±0.0 °2θ; Cu Kα1 radiation).

In some embodiments, the R-ketamine saccharin salt is a crystalline polymorphic form characterized by PXRD peaks at two or more, or three or more peaks selected from the group consisting of 7.8 °2θ, 13.8 °2θ, 15.3 °2θ, 15.6 °2θ, 16.0 °2θ, 16.6 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.0 °2θ, 26.2 °2θ, 26.8 °2θ, 28.7 °2θ, 32.9 °2θ, and 34.1 °2θ (±0.2 °2θ; ±0.1 °2θ; or 0.0 °2θ; Cu Kα1 radiation).

In some embodiments, the R-ketamine saccharin salt is a crystalline polymorphic form characterized by PXRD peaks at 7.8 °2θ, 13.8 °2θ, 15.3 °2θ, 15.6 °2θ, 16.0 °2θ, 16.6 °2θ, 19.4 °2θ, 19.7 °2θ, 20.0 °2θ, 20.2 °2θ, 20.5 °2θ, 22.0 °2θ, 22.2 °2θ, 23.5 °2θ, 25.2 °2θ, 26.0 °2θ, 26.2 °2θ, 26.8 °2θ, 28.7 °2θ, 32.9 °2θ, and 34.1 °2θ (±0.2 °2θ; ±0.1 °2θ; or ±0.0 °2θ; Cu Kα1 radiation).

In some embodiments, the R-ketamine saccharin salt is a crystalline polymorphic form characterized by one, two, three, four, five, six, seven, eight, nine, ten, eleven, twelve, thirteen, fourteen, fifteen or sixteen PXRD peaks selected from those set forth in Table 2.

TABLE 2
R-ketamine Saccharin Salt PXRD (±0.2 °2θ; ±0.1 °2θ;
or ±0.0 °2θ; Cu Kα1 radiation).

| Angle in | d-spacing | | |
|---|---|---|---|
| °2-theta | in Å | Intensity | Intensity % |
| 7.8 | 11.36 | w | 12 |
| 8.0 | 11.06 | w | 8 |
| 10.2 | 8.68 | w | 7 |
| 12.1 | 7.34 | vw | 5 |
| 13.8 | 6.40 | vs | 100 |
| 15.3 | 5.77 | m | 24 |
| 15.6 | 5.69 | s | 40 |
| 16.0 | 5.53 | s | 37 |
| 16.6 | 5.33 | w | 11 |
| 18.7 | 4.74 | vw | 5 |
| 19.0 | 4.67 | vw | 5 |
| 19.4 | 4.58 | m | 15 |
| 19.7 | 4.50 | w | 12 |
| 20.0 | 4.45 | m | 25 |
| 20.2 | 4.39 | m | 28 |
| 20.5 | 4.34 | s | 39 |
| 22.0 | 4.04 | s | 33 |
| 22.2 | 4.00 | m | 18 |
| 23.0 | 3.87 | vw | 4 |
| 23.5 | 3.78 | vs | 72 |
| 24.1 | 3.69 | w | 7 |
| 24.3 | 3.66 | w | 6 |
| 25.2 | 3.53 | m | 23 |
| 26.0 | 3.42 | w | 12 |
| 26.2 | 3.40 | w | 15 |
| 26.6 | 3.35 | vw | 4 |
| 26.8 | 3.33 | m | 27 |
| 27.9 | 3.20 | w | 5 |
| 28.1 | 3.17 | vw | 3 |
| 28.7 | 3.11 | w | 12 |
| 29.0 | 3.08 | vw | 5 |
| 29.8 | 3.00 | w | 6 |
| 30.0 | 2.98 | vw | 4 |
| 30.7 | 2.91 | vw | 5 |
| 30.9 | 2.89 | vw | 3 |
| 31.1 | 2.87 | w | 8 |
| 31.5 | 2.84 | vw | 3 |
| 32.4 | 2.77 | vw | 2 |
| 32.9 | 2.72 | w | 10 |
| 33.1 | 2.70 | w | 5 |
| 33.6 | 2.67 | w | 7 |
| 33.8 | 2.65 | vw | 5 |
| 34.1 | 2.63 | w | 10 |
| 35.0 | 2.56 | w | 8 |
| 35.4 | 2.53 | w | 8 |
| 35.7 | 2.51 | vw | 5 |
| 36.5 | 2.46 | vw | 4 |
| 36.9 | 2.43 | vw | 4 |
| 37.9 | 2.37 | vw | 2 |
| 38.4 | 2.34 | w | 6 |
| 38.8 | 2.32 | vw | 3 |
| 39.0 | 2.31 | vw | 2 |
| 39.3 | 2.29 | vw | 3 |
| 39.7 | 2.27 | vw | 2 |
| 39.9 | 2.26 | w | 8 |
| 40.8 | 2.21 | vw | 2 |

### R-Ketamine Fumarate Salt Form A

In one aspect, the present disclosure provides an R-ketamine fumarate salt. In some embodiments, the R-ketamine fumarate salt is amorphous. In some embodiments, the R-ketamine fumarate salt is crystalline. In some embodiments, the R-ketamine fumarate salt is a crystalline polymorphic Form A. In some embodiments, the R-ketamine fumarate salt Form A can be characterized by the PXRD peaks set forth below in Table 3.

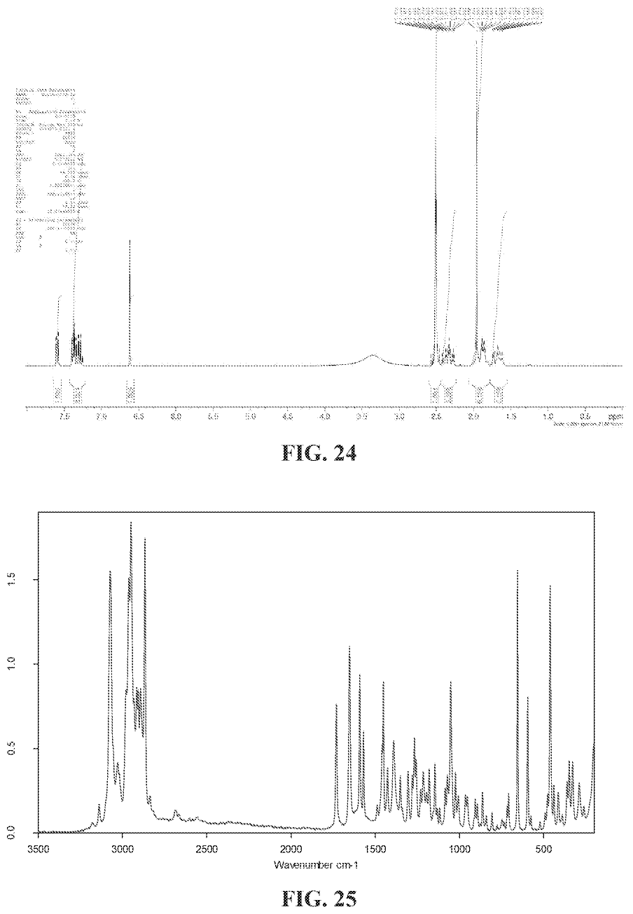

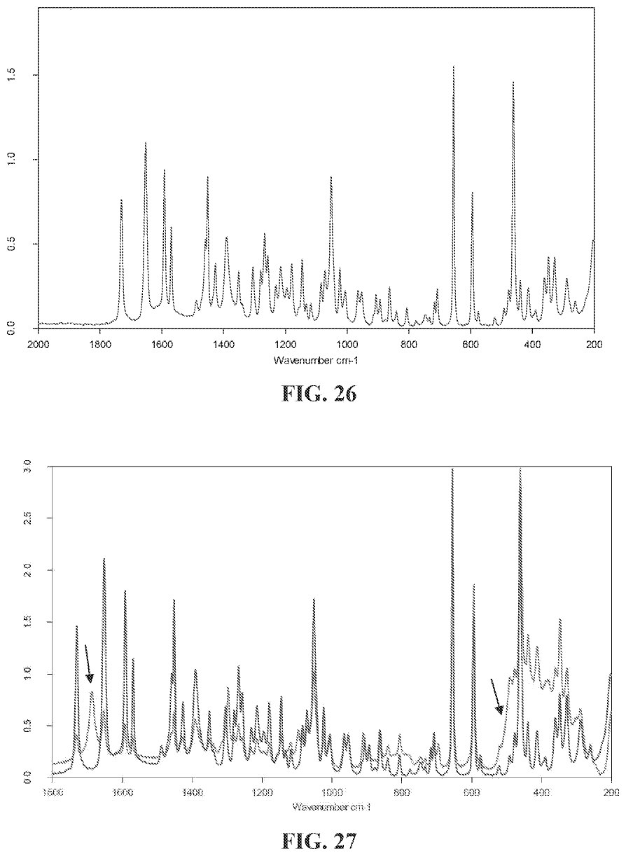

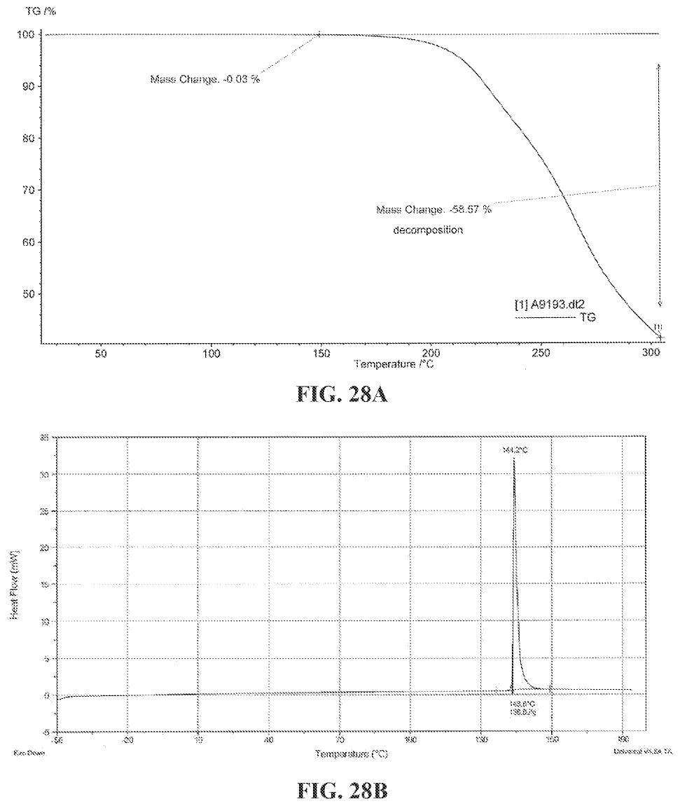

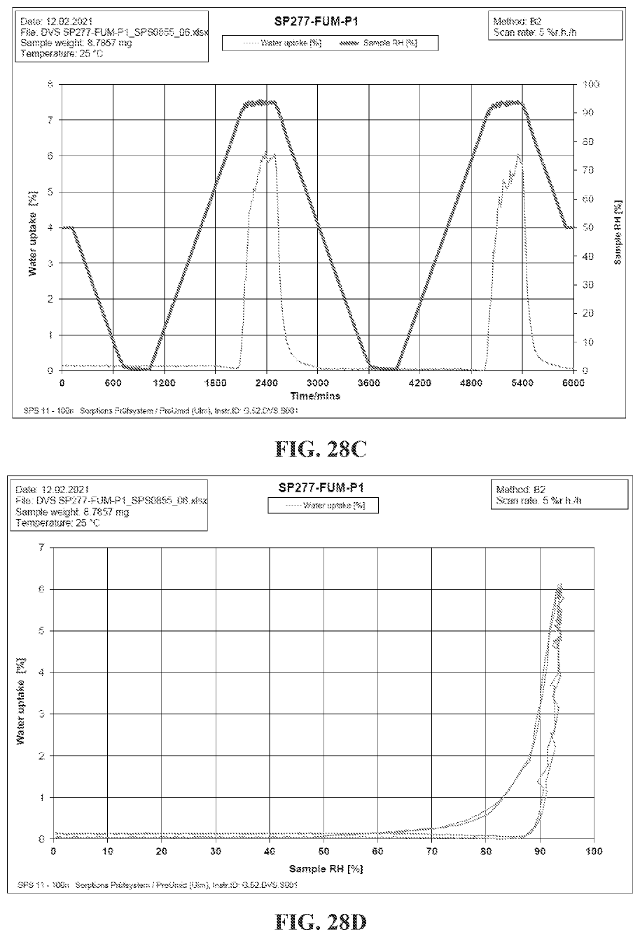

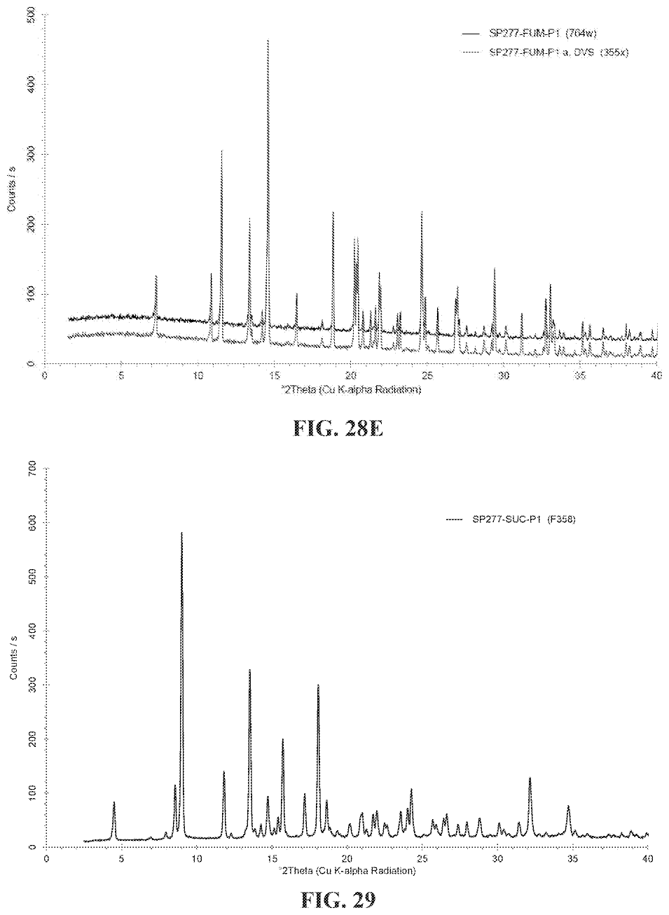

In some embodiments, the R-ketamine fumarate salt Form A is a crystalline polymorphic form characterized by PXRD spectrum substantially similar to that shown in FIG. 21. In some embodiments, the R-ketamine fumarate salt Form A is a crystalline polymorphic form characterized by FT-Raman spectrum substantially similar to that shown in FIG. 25 or 26. In some embodiments, the R-ketamine fumarate salt Form A is a crystalline polymorphic form characterized by TG-FTIR substantially similar to that shown in FIG. 28A. In some embodiments, the R-ketamine fumarate salt Form A is anhydrous.

In some embodiments, the R-ketamine fumarate salt Form A is a crystalline polymorphic form characterized by DSC having a melting peak at 144.2° C. In some embodiments, the R-ketamine fumarate salt Form A is a crystalline polymorphic form characterized by DSC having an associated enthalpy of 136.6 J/g.

In some embodiments, the R-ketamine fumarate salt Form A is characterized for ¹H-NMR substantially similar to FIG. 24.

In some embodiments, the R-ketamine fumarate salt Form A is a 1:1 R-ketamine:fumarate salt. In some embodiments, the R-ketamine fumarate salt Form A is a 2:1 R-ketamine:fumarate salt. In some embodiments, the R-ketamine fumarate salt Form A is a 1:2 R-ketamine:fumarate salt.

In some embodiments, the R-ketamine fumarate salt Form A is a crystalline polymorphic form characterized by a PXRD peak at 14.6 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 11.6 °2θ, and 14.6 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 11.6 °2θ, 13.4 °2θ, and 14.6 °2θ (±0.2 °2θ; ±0.1 °2θ; or 0.0 °2θ; Cu Kα1 radiation).

In some embodiments, the R-ketamine fumarate salt Form A is a crystalline polymorphic form characterized by PXRD peaks at 11.6 °2θ, 13.4 °2θ, and 14.6 °2θ (±0.2 °2θ; ±0.1 °2θ; or +0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 11.6 °2θ, 13.4 °2θ, 14.6 °2θ, and 18.9 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 11.6 °2θ, 13.4 °2θ, 14.6 °2θ, and 18.9 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 11.6 °2θ, 13.4 °2θ, 14.6 °2θ, and 24.6 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 11.6 °2θ, 13.4 °2θ, 14.6 °2θ, and 24.6 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 11.6 °2θ, 13.4 °2θ, 14.6 °2θ, 18.9 °2θ, and 24.6 °2θ (±0.2 °2θ; ±0.1 °2θ; or +0.0 °2θ; Cu Kα1 radiation).

In some embodiments, the R-ketamine fumarate salt Form A is a crystalline polymorphic form characterized by PXRD peaks at 11.6 °2θ, 13.4 °2θ, 14.6 °2θ, 18.9 °2θ, and 24.6 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 11.6 °2θ, 13.4 °2θ, 14.6 °2θ, 18.9 °2θ, 20.2 °2θ, and 24.6 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 11.6 °2θ, 13.4 °2θ, 14.6 °2θ, 18.9 °2θ, 20.2 °2θ, and 24.6 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 11.6 °2θ, 13.4 °2θ, 14.6 °2θ, 18.9 °2θ, 20.2 °2θ, 20.4 °2θ, and 24.6 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 11.6 °2θ, 13.4 °2θ, 14.6 °2θ, 18.9 °2θ, 20.2 °2θ, 20.4 °2θ, and 24.6 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 11.6 °2θ, 13.4 °2θ, 14.6 °2θ, 18.9 °2θ, 20.2 °2θ, 20.4 °2θ, 24.6 °2θ, and 29.4 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 11.6 °2θ, 13.4 °2θ, 14.6 °2θ, 18.9 °2θ, 20.2 °2θ, 20.4 °2θ, 24.6 °2θ, and 29.4 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 11.6 °2θ, 13.4 °2θ, 14.6 °2θ, 18.9 °2θ, 20.2 °2θ, 20.4 °2θ, 21.9 °2θ, 24.6 °2θ, 29.4 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 11.6 °2θ, 13.4 °2θ, 14.6 °2θ, 18.9 °2θ, 20.2 °2θ, 20.4 °2θ, 21.9 °2θ, 24.6 °2θ, 29.4 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 10.9 °2θ, 11.6 °2θ, 13.4 °2θ, 14.6 °2θ, 18.9 °2θ, 20.2 °2θ, 20.4 °2θ, 21.9 °2θ, 24.6 °2θ, and 29.4 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 10.9 °2θ, 11.6 °2θ, 13.4 °2θ, 14.6 °2θ, 18.9 °2θ, 20.2 °2θ, 20.4 °2θ, 21.9 °2θ, 24.6 °2θ, and 29.4 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 7.3 °2θ, 10.9 °2θ, 11.6 °2θ, 13.4 °2θ, 14.6 °2θ, 18.9 °2θ, 20.2 °2θ, 20.4 °2θ, 21.9 °2θ, 24.6 °2θ, and 29.4 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 7.3 °2θ, 10.9 °2θ, 11.6 °2θ, 13.4 °2θ, 14.6 °2θ, 18.9 °2θ, 20.2 °2θ, 20.4 °2θ, 21.9 °2θ, 24.6 °2θ, and 29.4 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 7.3 °2θ, 10.9 °2θ, 11.6 °2θ, 13.4 °2θ, 14.6 °2θ, 18.9 °2θ, 20.2 °2θ, 20.4 °2θ, 21.9 °2θ, 24.6 °2θ, 29.4 °2θ, and 33.0 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 7.3 °2θ, 10.9 °2θ, 11.6 °2θ, 13.4 °2θ, 14.6 °2θ, 18.9 °2θ, 20.2 °2θ, 20.4 °2θ, 21.9 °2θ, 24.6 °2θ, 29.4 °2θ, and 33.0 °2θ (L0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 7.3 °2θ, 10.9 °2θ, 11.6 °2θ, 13.4 °2θ, 14.6 °2θ, 18.9 °2θ, 20.2 °2θ, 20.4 °2θ, 21.9 °2θ, 24.6 °2θ, 26.9 °2θ, 29.4 °2θ, and 33.0 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 7.3 °2θ, 10.9 °2θ, 11.6 °2θ, 13.4 °2θ, 14.6 °2θ, 18.9 °2θ, 20.2 °2θ, 20.4 °2θ, 21.9 °2θ, 24.6 °2θ, 26.9 °2θ, 29.4 °2θ, and 33.0 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 7.3 °2θ, 10.9 °2θ, 11.6 °2θ, 13.4 °2θ, 14.6 °2θ, 16.5 °2θ, 18.9 °2θ, 20.2 °2θ, 20.4 °2θ, 21.9 °2θ, 24.6 °2θ, 26.9 °2θ, 29.4 °2θ, and 33.0 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 7.3 °2θ, 10.9 °2θ, 11.6 °2θ, 13.4 °2θ, 14.6 °2θ, 16.5 °2θ, 18.9 °2θ, 20.2 °2θ, 20.4 °2θ, 21.9 °2θ, 24.6 °2θ, 26.9 °2θ, 29.4 °2θ, and 33.0 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 7.3 °2θ, 10.9 °2θ, 11.6 °2θ, 13.4 °2θ, 14.6 °2θ, 16.5 °2θ, 18.9 °2θ, 20.2 °2θ, 20.4 °2θ, 21.9 °2θ, 24.6 °2θ, 24.9 °2θ, 26.9 °2θ, 29.4 °2θ, and 33.0 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 7.3 °2θ, 10.9 °2θ, 11.6 °2θ, 13.4 °2θ, 14.6 °2θ, 16.5 °2θ, 18.9 °2θ, 20.2 °2θ, 20.4 °2θ, 21.9 °2θ, 24.6 °2θ, 24.9 °2θ, 26.9 °2θ, 29.4 °2θ, and 33.0 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at two or more, or three or more peaks selected from the group consisting of 7.3 °2θ, 10.9 °2θ, 11.6 °2θ, 13.4 °2θ, 14.6 °2θ, 16.5 °2θ, 18.9 °2θ, 20.2 °2θ, 20.4 °2θ, 21.9 °2θ, 24.6 °2θ, 24.9 °2θ, 26.9 °2θ, 29.4 °2θ, 32.7 °2θ, and 33.0 °2θ (±0.2 °2θ; ±0.1 °2θ; or ±0.0 °2θ; Cu Kα1 radiation).

In some embodiments, the R-ketamine fumarate salt Form A is a crystalline polymorphic form characterized by PXRD peaks at 7.3 °2θ, 10.9 °2θ, 11.6 °2θ, 13.4 °2θ, 14.6 °2θ, 16.5 °2θ, 18.9 °2θ, 20.2 °2θ, 20.4 °2θ, 21.9 °2θ, 24.6 °2θ, 24.9 °2θ, 26.9 °2θ, 29.4 °2θ, 32.7 °2θ, and 33.0 °2θ (±0.2 °2θ; ±0.1 °2θ; or ±0.0 °2θ; Cu Kα1 radiation).

In some embodiments, the R-ketamine fumarate salt Form A is characterized by one, two, three, four, five, six, seven, eight, nine, ten, eleven, twelve, thirteen, fourteen, fifteen or sixteen PXRD peaks selected from those set forth in Table 3.

TABLE 3
R-ketamine Fumarate Salt Form A PXRD (±0.2 °2θ; ±0.1 °2θ;
or ±0.0 °2θ; Cu Kα1 radiation).

| Angle in | d-spacing | | |
|---|---|---|---|
| °2-theta | in Å | Intensity | Intensity % |
| 7.3 | 12.16 | m | 27 |
| 10.9 | 8.13 | m | 28 |
| 11.6 | 7.65 | vs | 71 |
| 13.4 | 6.62 | s | 50 |
| 14.2 | 6.23 | w | 14 |
| 14.6 | 6.07 | vs | 100 |
| 16.5 | 5.38 | m | 21 |
| 18.1 | 4.89 | w | 10 |
| 18.9 | 4.70 | s | 49 |
| 20.2 | 4.39 | s | 40 |
| 20.4 | 4.35 | s | 30 |
| 20.8 | 4.27 | w | 14 |
| 21.3 | 4.17 | w | 14 |
| 21.6 | 4.11 | m | 16 |
| 21.9 | 4.06 | m | 29 |
| 22.8 | 3.90 | w | 8 |
| 23.0 | 3.86 | w | 13 |
| 23.2 | 3.83 | w | 13 |
| 24.6 | 3.61 | s | 47 |
| 24.9 | 3.58 | m | 19 |
| 25.1 | 3.55 | w | 6 |
| 25.7 | 3.47 | m | 15 |
| 26.9 | 3.31 | m | 22 |
| 27.6 | 3.23 | w | 8 |
| 28.1 | 3.17 | w | 6 |
| 28.7 | 3.11 | w | 8 |
| 29.2 | 3.06 | w | 9 |
| 29.4 | 3.04 | s | 30 |
| 29.7 | 3.00 | w | 5 |
| 30.1 | 2.96 | w | 8 |
| 31.2 | 2.87 | w | 13 |
| 31.7 | 2.82 | vw | 4 |
| 32.1 | 2.79 | w | 5 |
| 32.7 | 2.73 | m | 19 |
| 33.0 | 2.71 | m | 24 |
| 33.2 | 2.69 | w | 11 |
| 33.7 | 2.66 | w | 6 |
| 33.9 | 2.64 | w | 6 |
| 34.7 | 2.59 | vw | 5 |
| 35.1 | 2.55 | w | 10 |
| 35.6 | 2.52 | w | 9 |
| 36.5 | 2.46 | w | 7 |
| 36.7 | 2.45 | vw | 4 |
| 37.0 | 2.43 | vw | 4 |
| 37.5 | 2.40 | vw | 3 |
| 37.7 | 2.38 | vw | 4 |
| 38.0 | 2.37 | w | 9 |
| 38.2 | 2.35 | w | 7 |
| 38.5 | 2.33 | vw | 4 |
| 38.9 | 2.31 | w | 6 |
| 39.7 | 2.27 | w | 7 |
| 40.1 | 2.25 | w | 9 |
| 40.8 | 2.21 | w | 9 |

### R-Ketamine Fumarate Salt Form B

In one aspect, the present disclosure provides an R-ketamine fumarate salt Form B. In some embodiments, the R-ketamine fumarate salt is amorphous. In some embodiments, the R-ketamine fumarate salt is crystalline. In some embodiments, the R-ketamine fumarate salt is a crystalline polymorphic form. In some embodiments, the R-ketamine fumarate salt is a crystalline polymorphic form characterized by characterized by the PXRD peaks set forth below in Table 4.

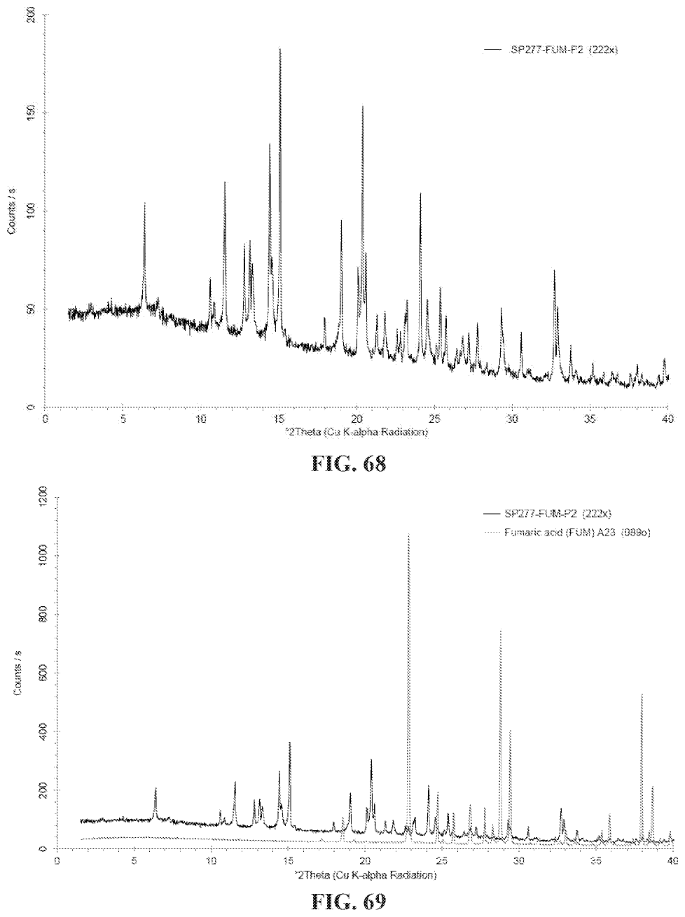

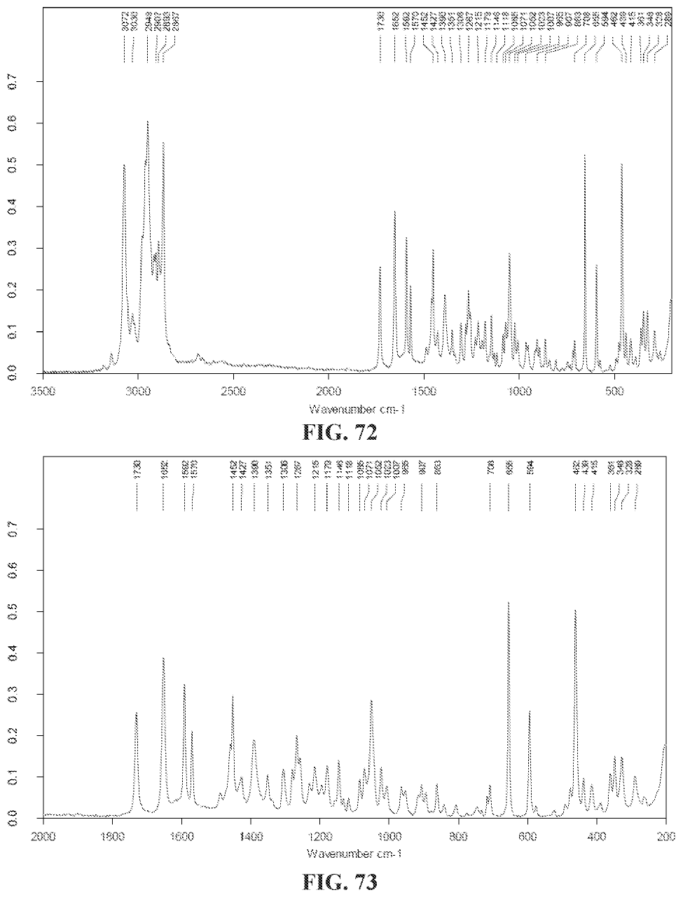

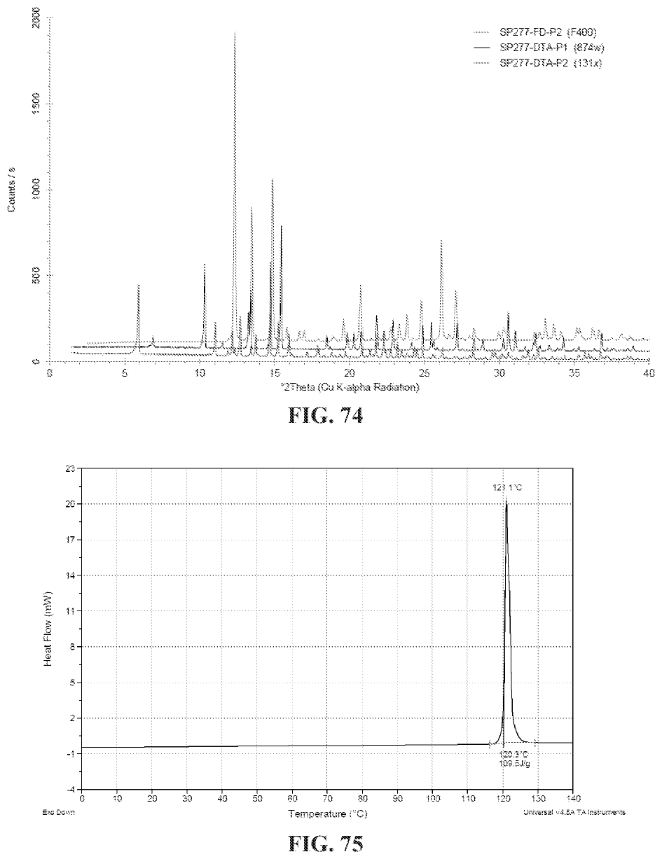

In some embodiments, the R-ketamine fumarate salt Form B is a crystalline polymorphic form characterized by PXRD spectrum substantially similar to that shown in FIG. 68. In some embodiments, the R-ketamine fumarate salt Form B is a crystalline polymorphic form characterized by FT-Raman spectrum substantially similar to that shown in FIG. 72 or 73.

In some embodiments, the R-ketamine fumarate salt Form B is a crystalline polymorphic form characterized by characterized for ¹H-NMR substantially similar to FIG. 71.

In some embodiments, the R-ketamine fumarate salt Form B is a 1:1 R-ketamine:fumarate salt. In some embodiments, the R-ketamine fumarate salt Form B is a 2:1 R-ketamine:fumarate salt. In some embodiments, the R-ketamine fumarate salt Form B is a 1:2 R-ketamine:fumarate salt.

In some embodiments, the R-ketamine fumarate salt Form B is a crystalline polymorphic form characterized by a PXRD peak at 15.1 °2θ (±0.2 °2θ; ±0.1 °2θ; or 0.0 °2θ; Cu Kα1 radiation).

In some embodiments, the R-ketamine fumarate salt Form B is a crystalline polymorphic form characterized by PXRD peaks at 15.1 °2θ, and 20.4 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 14.5 °2θ, 15.1 °2θ, and 20.4 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at 14.5 °2θ, 15.1 °2θ, and 20.4 °2θ (±0.2 °2θ; ±0.1 °2θ; or +0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 11.5 °2θ, 14.5 °2θ, 15.1 °2θ, and 20.4 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at 11.5 °2θ, 14.5 °2θ, 15.1 °2θ, and 20.4 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 11.5 °2θ, 14.5 °2θ, 15.1 °2θ, 20.4 °2θ, and 24.1 °2θ (±0.2 °2θ; ±0.1 °2θ; or +0.0 °2θ; Cu Kα1 radiation).

In some embodiments, the R-ketamine fumarate salt Form B is a crystalline polymorphic form characterized by PXRD peaks at 11.5 °2θ, 14.5 °2θ, 15.1 °2θ, 20.4 °2θ, and 24.1 °2θ (L0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 6.4 °2θ, 11.5 °2θ, 14.5 °2θ, 15.1 °2θ, 20.4 °2θ, and 24.1 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at 6.4 °2θ, 11.5 °2θ, 14.5 °2θ, 15.1 °2θ, 20.4 °2θ, and 24.1 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 6.4 °2θ, 11.5 °2θ, 14.5 °2θ, 15.1 °2θ, 19.0 °2θ, 20.4 °2θ, and 24.1 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at 6.4 °2θ, 11.5 °2θ, 14.5 °2θ, 15.1 °2θ, 19.0 °2θ, 20.4 °2θ, and 24.1 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 6.4 °2θ, 11.5 °2θ, 13.1 °2θ, 14.5 °2θ, 15.1 °2θ, 19.0 °2θ, 20.4 °2θ, and 24.1 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at 6.4 °2θ, 11.5 °2θ, 13.1 °2θ, 14.5 °2θ, 15.1 °2θ, 19.0 °2θ, 20.4 °2θ, and 24.1 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 6.4 °2θ, 11.5 °2θ, 12.8 °2θ, 13.1 °2θ, 14.5 °2θ, 15.1 °2θ, 19.0 °2θ, 20.4 °2θ, and 24.1 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at 6.4 °2θ, 11.5 °2θ, 12.8 °2θ, 13.1 °2θ, 14.5 °2θ, 15.1 °2θ, 19.0 °2θ, 20.4 °2θ, and 24.1 °2θ (±0.2 °2θ; ±0.1 °2θ; or 0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 6.4 °2θ, 11.5 °2θ, 12.8 °2θ, 13.1 °2θ, 14.5 °2θ, 15.1 °2θ, 19.0 °2θ, 20.4 °2θ, 20.6 °2θ, and 24.1 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at 6.4 °2θ, 11.5 °2θ, 12.8 °2θ, 13.1 °2θ, 14.5 °2θ, 15.1 °2θ, 19.0 °2θ, 20.4 °2θ, 20.6 °2θ, and 24.1 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 6.4 °2θ, 11.5 °2θ, 12.8 °2θ, 13.1 °2θ, 13.4 °2θ, 14.5 °2θ, 15.1 °2θ, 19.0 °2θ, 20.4 °2θ, 20.6 °2θ, and 24.1 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at 6.4 °2θ, 11.5 °2θ, 12.8 °2θ, 13.1 °2θ, 13.4 °2θ, 14.5 °2θ, 15.1 °2θ, 19.0 °2θ, 20.4 °2θ, 20.6 °2θ, and 24.1 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 6.4 °2θ, 11.5 °2θ, 12.8 °2θ, 13.1 °2θ, 13.4 °2θ, 14.5 °2θ, 15.1 °2θ, 19.0 °2θ, 20.4 °2θ, 20.6 °2θ, 24.1 °2θ, and 32.7 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at 6.4 °2θ, 11.5 °2θ, 12.8 °2θ, 13.1 °2θ, 13.4 °2θ, 14.5 °2θ, 15.1 °2θ, 19.0 °2θ, 20.4 °2θ, 20.6 °2θ, 24.1 °2θ, and 32.7 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 6.4 °2θ, 11.5 °2θ, 12.8 °2θ, 13.1 °2θ, 13.4 °2θ, 14.5 °2θ, 15.1 °2θ, 19.0 °2θ, 20.1 °2θ, 20.4 °2θ, 20.6 °2θ, 24.1 °2θ, and 32.7 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at 6.4 °2θ, 11.5 °2θ, 12.8 °2θ, 13.1 °2θ, 13.4 °2θ, 14.5 °2θ, 15.1 °2θ, 19.0 °2θ, 20.1 °2θ, 20.4 °2θ, 20.6 °2θ, 24.1 °2θ, and 32.7 °2θ (±0.2 °2θ; ±0.1 °2θ; or +0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 6.4 °2θ, 10.6 °2θ, 11.5 °2θ, 12.8 °2θ, 13.1 °2θ, 13.4 °2θ, 14.5 °2θ, 15.1 °2θ, 19.0 °2θ, 20.1 °2θ, 20.4 °2θ, 20.6 °2θ, 24.1 °2θ, and 32.7 °2θ (±0.2 °2θ; ±0.1 °2θ; or 0.0 °2θ; Cu Kα1 radiation).

In some embodiments, the R-ketamine fumarate salt Form B is a crystalline polymorphic form characterized by PXRD peaks at 6.4 °2θ, 10.6 °2θ, 11.5 °2θ, 12.8 °2θ, 13.1 °2θ, 13.4 °2θ, 14.5 °2θ, 15.1 °2θ, 19.0 °2θ, 20.1 °2θ, 20.4 °2θ, 20.6 °2θ, 24.1 °2θ, and 32.7 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 6.4 °2θ, 10.6 °2θ, 11.5 °2θ, 12.8 °2θ, 13.1 °2θ, 13.4 °2θ, 14.5 °2θ, 15.1 °2θ, 19.0 °2θ, 20.1 °2θ, 20.4 °2θ, 20.6 °2θ, 24.1 °2θ, 25.4 °2θ, and 32.7 °2θ (±0.2 °2θ; ±0.1 °2θ; or +0.0 °2θ; Cu Kα1 radiation).

In some embodiments, the R-ketamine fumarate salt Form B is a crystalline polymorphic form characterized by PXRD peaks at 6.4 °2θ, 10.6 °2θ, 11.5 °2θ, 12.8 °2θ, 13.1 °2θ, 13.4 °2θ, 14.5 °2θ, 15.1 °2θ, 19.0 °2θ, 20.1 °2θ, 20.4 °2θ, 20.6 °2θ, 24.1 °2θ, 25.4 °2θ, and 32.7 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at two or more, or three or more peaks selected from the group consisting of 6.4 °2θ, 7.2 °2θ, 10.6 °2θ, 11.5 °2θ, 12.8 °2θ, 13.1 °2θ, 13.4 °2θ, 14.5 °2θ, 15.1 °2θ, 19.0 °2θ, 20.1 °2θ, 20.4 °2θ, 20.6 °2θ, 24.1 °2θ, 25.4 °2θ, and 32.7 °2θ (±0.2 °2θ; ±0.1 °2θ; or ±0.0 °2θ; Cu Kα1 radiation).

In some embodiments, the R-ketamine fumarate salt Form B is a crystalline polymorphic form characterized by PXRD peaks at 6.4 °2θ, 7.2 °2θ, 10.6 °2θ, 11.5 °2θ, 12.8 °2θ, 13.1 °2θ, 13.4 °2θ, 14.5 °2θ, 15.1 °2θ, 19.0 °2θ, 20.1 °2θ, 20.4 °2θ, 20.6 °2θ, 24.1 °2θ, 25.4 °2θ, and 32.7 °2θ (±0.2 °2θ; ±0.1 °2θ; or ±0.0 °2θ; Cu Kα1 radiation).

In some embodiments, the R-ketamine fumarate salt Form B is characterized by one, two, three, four, five, six, seven, eight, nine, ten, eleven, twelve, thirteen, fourteen, fifteen or sixteen PXRD peaks selected from those set forth in Table 4.

TABLE 4
R-ketamine Fumarate Salt Form B PXRD (±0.2 °2θ; ±0.1 °2θ;
or ±0.0 °2θ; Cu Kα1 radiation).

| Angle in | d-spacing | | |
|---|---|---|---|
| °2-theta | in Å | Intensity | Intensity % |
| 6.4 | 13.84 | s | 57 |
| 7.2 | 12.21 | s | 31 |
| 10.6 | 8.34 | s | 36 |
| 10.8 | 8.15 | m | 29 |
| 11.5 | 7.66 | s | 63 |
| 12.8 | 6.91 | s | 46 |
| 13.1 | 6.73 | s | 47 |
| 13.4 | 6.62 | s | 38 |
| 14.5 | 6.13 | vs | 74 |
| 15.1 | 5.87 | vs | 100 |
| 18.0 | 4.94 | m | 25 |
| 19.0 | 4.66 | s | 52 |
| 20.1 | 4.41 | s | 37 |
| 20.4 | 4.35 | vs | 84 |
| 20.6 | 4.30 | s | 43 |
| 21.3 | 4.17 | m | 26 |
| 21.8 | 4.07 | m | 27 |
| 22.6 | 3.93 | m | 22 |
| 23.2 | 3.83 | m | 28 |
| 24.1 | 3.69 | s | 60 |
| 24.6 | 3.62 | s | 30 |
| 25.4 | 3.50 | s | 34 |
| 25.8 | 3.46 | m | 26 |
| 26.4 | 3.37 | m | 17 |
| 26.8 | 3.32 | m | 20 |
| 27.2 | 3.28 | m | 21 |
| 27.8 | 3.21 | m | 24 |
| 28.4 | 3.14 | w | 13 |
| 29.3 | 3.04 | m | 26 |
| 29.8 | 3.00 | w | 12 |
| 30.6 | 2.92 | m | 21 |
| 31.0 | 2.88 | w | 11 |
| 32.2 | 2.78 | w | 9 |
| 32.7 | 2.73 | s | 38 |
| 32.9 | 2.72 | m | 28 |
| 33.8 | 2.65 | m | 18 |
| 34.1 | 2.63 | w | 10 |
| 35.2 | 2.55 | w | 12 |
| 35.9 | 2.50 | w | 10 |
| 36.4 | 2.46 | w | 10 |
| 36.8 | 2.44 | w | 9 |
| 37.6 | 2.39 | w | 9 |
| 38.0 | 2.36 | w | 12 |
| 38.3 | 2.35 | w | 10 |
| 39.4 | 2.28 | w | 9 |
| 39.8 | 2.26 | w | 14 |
| 40.7 | 2.22 | m | 19 |

### R-Ketamine Succinate Salt

In one aspect, the present disclosure provides an R-ketamine succinate salt. In some embodiments, the R-ketamine succinate salt is amorphous. In some embodiments, the R-ketamine succinate salt is crystalline. In some embodiments, the R-ketamine succinate salt is a crystalline polymorphic form. In some embodiments, the R-ketamine succinate salt is a crystalline polymorphic form characterized by the PXRD peaks set forth below in Table 5.

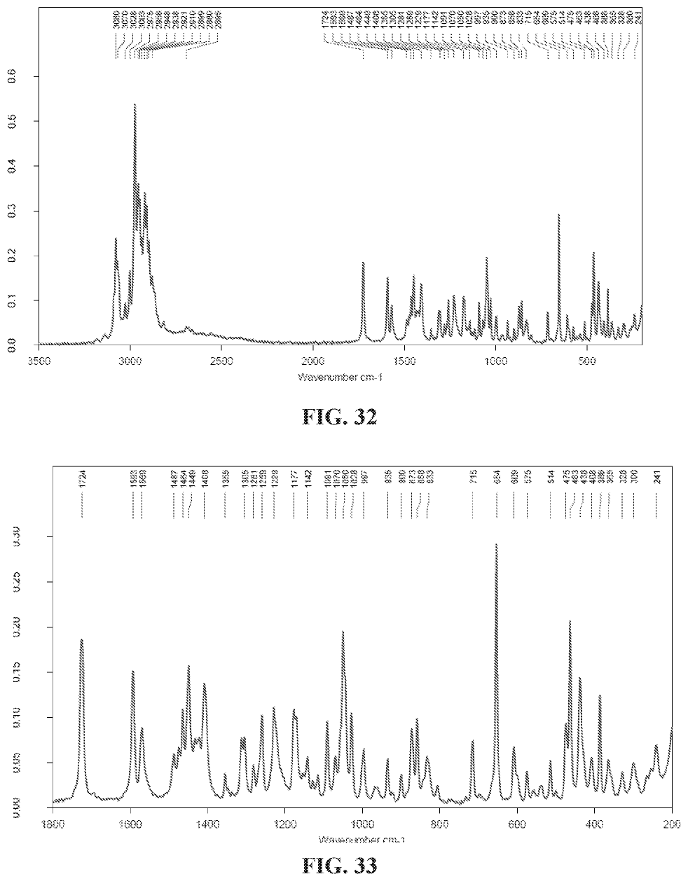

In some embodiments, the R-ketamine succinate salt is a crystalline polymorphic form characterized by PXRD spectrum is substantially similar to that shown in FIG. 29. In some embodiments, the R-ketamine succinate salt is a crystalline polymorphic form characterized by FT-Raman spectrum is substantially similar to that shown in FIG. 32. In some embodiments, the R-ketamine succinate salt is a hydrate. In some embodiments, the R-ketamine succinate salt is anhydrous.

In some embodiments, the R-ketamine succinate salt is a crystalline polymorphic form characterized by characterized by ¹H-NMR substantially similar to FIG. 31.

In some embodiments, the R-ketamine succinate salt is a 1:1 R-ketamine:succinate salt. In some embodiments, the R-ketamine succinate salt is a 2:1 R-ketamine:succinate salt. In some embodiments, the R-ketamine succinate salt is a 1:2 R-ketamine:succinate salt.

In some embodiments, the R-ketamine succinate salt is a crystalline polymorphic form characterized by a PXRD peak at 9.0 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 9.0 °2θ, and 13.5 °2θ (10.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 9.0 °2θ, 13.5 °2θ, and 18.1 °2θ (±0.2 °2θ; ±0.1 °2θ; or 0.0 °2θ; Cu Kα1 radiation).

In some embodiments, the R-ketamine succinate salt is a crystalline polymorphic form characterized by PXRD peaks at 9.0 °2θ, 13.5 °2θ, and 18.1 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 9.0 °2θ, 13.5 °2θ, 15.7 °2θ, and 18.1 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 9.0 °2θ, 13.5 °2θ, 15.7 °2θ, and 18.1 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 9.0 °2θ, 11.8 °2θ, 13.5 °2θ, 15.7 °2θ, and 18.1 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 9.0 °2θ, 11.8 °2θ, 13.5 °2θ, 15.7 °2θ, and 18.1 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 9.0 °2θ, 11.8 °2θ, 13.5 °2θ, 15.7 °2θ, 18.1 °2θ, and 32.2 °2θ (±0.2 °2θ; ±0.1 °2θ; or +0.0 °2θ; Cu Kα1 radiation).

In some embodiments, the R-ketamine succinate salt is a crystalline polymorphic form characterized by PXRD peaks at 9.0 °2θ, 11.8 °2θ, 13.5 °2θ, 15.7 °2θ, 18.1 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 8.6 °2θ, 9.0 °2θ, 11.8 °2θ, 13.5 °2θ, 15.7 °2θ, 18.1 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 8.6 °2θ, 9.0 °2θ, 11.8 °2θ, 13.5 °2θ, 15.7 °2θ, 18.1 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 8.6 °2θ, 9.0 °2θ, 11.8 °2θ, 13.5 °2θ, 15.7 °2θ, 18.1 °2θ, 24.3 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 8.6 °2θ, 9.0 °2θ, 11.8 °2θ, 13.5 °2θ, 15.7 °2θ, 18.1 °2θ, 24.3 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 8.6 °2θ, 9.0 °2θ, 11.8 °2θ, 13.5 °2θ, 15.7 °2θ, 17.2 °2θ, 18.1 °2θ, 24.3 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 8.6 °2θ, 9.0 °2θ, 11.8 °2θ, 13.5 °2θ, 15.7 °2θ, 17.2 °2θ, 18.1 °2θ, 24.3 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 8.6 °2θ, 9.0 °2θ, 11.8 °2θ, 13.5 °2θ, 14.7 °2θ, 15.7 °2θ, 17.2 °2θ, 18.1 °2θ, 24.3 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 8.6 °2θ, 9.0 °2θ, 11.8 °2θ, 13.5 °2θ, 14.7 °2θ, 15.7 °2θ, 17.2 °2θ, 18.1 °2θ, 24.3 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 8.6 °2θ, 9.0 °2θ, 11.8 °2θ, 13.5 °2θ, 14.7 °2θ, 15.7 °2θ, 17.2 °2θ, 18.1 °2θ, 18.7 °2θ, 24.3 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 8.6 °2θ, 9.0 °2θ, 11.8 °2θ, 13.5 °2θ, 14.7 °2θ, 15.7 °2θ, 17.2 °2θ, 18.1 °2θ, 18.7 °2θ, 24.3 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 4.5 °2θ, 8.6 °2θ, 9.0 °2θ, 11.8 °2θ, 13.5 °2θ, 14.7 °2θ, 15.7 °2θ, 17.2 °2θ, 18.1 °2θ, 18.7 °2θ, 24.3 °2θ, and 32.2 °2θ (10.2 °2θ; 10.1 °2θ; or ±0.0 °2θ; Cu Kα1 radiation).

In some embodiments, the R-ketamine succinate salt is a crystalline polymorphic form characterized by PXRD peaks at 4.5 °2θ, 8.6 °2θ, 9.0 °2θ, 11.8 °2θ, 13.5 °2θ, 14.7 °2θ, 15.7 °2θ, 17.2 °2θ, 18.1 °2θ, 18.7 °2θ, 24.3 °2θ, and 32.2 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 4.5 °2θ, 8.6 °2θ, 9.0 °2θ, 11.8 °2θ, 13.5 °2θ, 14.7 °2θ, 15.7 °2θ, 17.2 °2θ, 18.1 °2θ, 18.7 °2θ, 24.3 °2θ, 32.2 °2θ, and 34.7 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 4.5 °2θ, 8.6 °2θ, 9.0 °2θ, 11.8 °2θ, 13.5 °2θ, 14.7 °2θ, 15.7 °2θ, 17.2 °2θ, 18.1 °2θ, 18.7 °2θ, 24.3 °2θ, 32.2 °2θ, and 34.7 °2θ (10.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 4.5 °2θ, 8.6 °2θ, 9.0 °2θ, 11.8 °2θ, 13.5 °2θ, 14.7 °2θ, 15.7 °2θ, 17.2 °2θ, 18.1 °2θ, 18.7 °2θ, 24.0 °2θ, 24.3 °2θ, 32.2 °2θ, and 34.7 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 4.5 °2θ, 8.6 °2θ, 9.0 °2θ, 11.8 °2θ, 13.5 °2θ, 14.7 °2θ, 15.7 °2θ. 17.2 °2θ, 18.1 °2θ, 18.7 °2θ, 24.0 °2θ, 24.3 °2θ, 32.2 °2θ, and 34.7 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 4.5 °2θ, 8.6 °2θ, 9.0 °2θ, 11.8 °2θ, 13.5 °2θ, 14.7 °2θ, 15.7 °2θ, 17.2 °2θ, 18.1 °2θ, 18.7 °2θ, 22.0 °2θ, 24.0 °2θ, 24.3 °2θ, 32.2 °2θ, and 34.7 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 4.5 °2θ, 8.6 °2θ, 9.0 °2θ, 11.8 °2θ, 13.5 °2θ, 14.7 °2θ, 15.7 °2θ. 17.2 °2θ, 18.1 °2θ, 18.7 °2θ, 22.0 °2θ, 24.0 °2θ, 24.3 °2θ, 32.2 °2θ, and 34.7 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at two or more, or three or more peaks selected from the group consisting of 4.5 °2θ, 8.6 °2θ, 9.0 °2θ, 11.8 °2θ, 13.5 °2θ, 14.7 °2θ, 15.7 °2θ, 17.2 °2θ, 18.1 °2θ, 18.7 °2θ, 22.0 °2θ, 24.0 °2θ, 24.3 °2θ, 26.6 °2θ, 32.2 °2θ, and 34.7 °2θ (±0.2 °2θ; ±0.1 °2θ; or ±0.0 °2θ; Cu Kα1 radiation).

In some embodiments, the R-ketamine succinate salt is a crystalline polymorphic form characterized by PXRD peaks at 4.5 °2θ, 8.6 °2θ, 9.0 °2θ, 11.8 °2θ, 13.5 °2θ, 14.7 °2θ, 15.7 °2θ, 17.2 °2θ, 18.1 °2θ, 18.7 °2θ, 22.0 °2θ, 24.0 °2θ, 24.3 °2θ, 26.6 °2θ, 32.2 °2θ, and 34.7 °2θ (±0.2 °2θ; ±0.1 °2θ; or ±0.0 °2θ; Cu Kα1 radiation).

In some embodiments, the R-ketamine succinate salt is a crystalline polymorphic form characterized by one, two, three, four, five, six, seven, eight, nine, ten, eleven, twelve, thirteen, fourteen, fifteen or sixteen PXRD peaks selected from those set forth in Table 5.

TABLE 5
R-ketamine Succinate Salt PXRD (±0.2 °2θ; ±0.1 °2θ;
or ±0.0 °2θ; Cu Kα1 radiation).

| Angle in | d-spacing | | |
|---|---|---|---|
| °2-theta | in Å | Intensity | Intensity % |
| 4.5 | 19.6 | w | 14 |
| 6.9 | 12.74 | vw | 3 |
| 8.0 | 11.11 | vw | 5 |
| 8.6 | 10.32 | m | 20 |
| 9.0 | 9.80 | vs | 100 |
| 11.8 | 7.48 | m | 24 |
| 12.3 | 7.20 | vw | 5 |
| 13.5 | 6.54 | s | 56 |
| 13.9 | 6.38 | w | 6 |
| 14.3 | 6.20 | w | 7 |
| 14.7 | 6.01 | m | 16 |
| 15.2 | 5.84 | w | 6 |
| 15.4 | 5.75 | w | 10 |
| 15.7 | 5.63 | s | 34 |
| 16.7 | 5.32 | vw | 4 |
| 17.2 | 5.16 | m | 17 |
| 18.1 | 4.90 | s | 52 |
| 18.7 | 4.75 | w | 15 |
| 18.9 | 4.69 | w | 6 |
| 19.4 | 4.58 | w | 5 |
| 20.2 | 4.40 | w | 7 |
| 21.0 | 4.23 | w | 11 |
| 21.3 | 4.17 | w | 6 |
| 21.7 | 4.09 | w | 11 |
| 22.0 | 4.04 | w | 12 |
| 22.5 | 3.95 | w | 8 |
| 23.6 | 3.77 | w | 11 |
| 24.0 | 3.70 | w | 12 |
| 24.3 | 3.66 | m | 19 |
| 25.1 | 3.54 | vw | 4 |
| 25.7 | 3.46 | w | 9 |
| 25.9 | 3.44 | w | 7 |
| 26.4 | 3.37 | w | 9 |
| 26.6 | 3.35 | w | 11 |
| 27.4 | 3.25 | w | 7 |
| 28.0 | 3.19 | w | 8 |
| 28.8 | 3.10 | w | 9 |
| 29.5 | 3.03 | vw | 4 |
| 30.1 | 2.96 | w | 8 |
| 30.4 | 2.94 | w | 6 |
| 30.7 | 2.91 | vw | 4 |
| 31.4 | 2.84 | w | 8 |
| 32.2 | 2.78 | m | 22 |
| 32.8 | 2.73 | vw | 4 |
| 33.2 | 2.69 | vw | 5 |
| 34.1 | 2.63 | vw | 5 |
| 34.7 | 2.58 | w | 13 |
| 35.2 | 2.55 | w | 5 |
| 35.7 | 2.51 | vw | 4 |
| 36.0 | 2.50 | vw | 4 |
| 37.4 | 2.40 | vw | 4 |
| 37.7 | 2.38 | vw | 4 |
| 38.3 | 2.35 | vw | 5 |
| 38.9 | 2.31 | w | 5 |
| 39.3 | 2.29 | vw | 4 |
| 40.0 | 2.25 | vw | 5 |
| 40.4 | 2.23 | vw | 3 |

### R-Ketamine Sulfate Salt

In one aspect, the present disclosure provides an R-ketamine sulfate salt. In some embodiments, the R-ketamine sulfate salt is amorphous. In some embodiments, the R-ketamine sulfate salt is crystalline. In some embodiments, the R-ketamine sulfate salt is a crystalline polymorphic form. In some embodiments, the R-ketamine sulfate salt is a crystalline polymorphic form characterized by the PXRD peaks set forth below in Table 6.

In some embodiments, the R-ketamine sulfate salt is a crystalline polymorphic form characterized by a PXRD spectrum substantially similar to that shown in FIG. 36.

In some embodiments, the R-ketamine sulfate salt is a 1:1 R-ketamine:sulfate salt. In some embodiments, the R-ketamine sulfate salt is a 2:1 R-ketamine:sulfate salt. In some embodiments, the R-ketamine sulfate salt is a 1:2 R-ketamine:sulfate salt.

In some embodiments, the R-ketamine sulfate salt is a crystalline polymorphic form characterized by a PXRD peak at 19.8 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at 19.8 °2θ and 22.5 °2θ, (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at two or more, or three or more peaks selected from the group consisting of 19.8 °2θ, 22.5 °2θ, and 26.1 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at 19.8 °2θ, 22.5 °2θ, and 26.1 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at two or more, or three or more peaks selected from the group consisting of 19.8 °2θ, 20.1 °2θ, 22.5 °2θ, and 26.1 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at 19.8 °2θ, 20.1 °2θ, 22.5 °2θ, and 26.1 °2θ (±0.2 °2θ; ±0.1 °2θ; or +0.0 °2θ; Cu Kα1 radiation).

In some embodiments, the R-ketamine sulfate salt is a crystalline polymorphic form characterized by PXRD peaks at two or more, or three or more peaks selected from the group consisting of 19.8 °2θ, 20.1 °2θ, 22.5 °2θ, 24.5 °2θ, and 26.1 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at 19.8 °2θ, 20.1 °2θ, 22.5 °2θ, 24.5 °2θ, and 26.1 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at two or more, or three or more peaks selected from the group consisting of 19.8 °2θ, 20.1 °2θ, 22.5 °2θ, 24.5 °2θ, 26.1 °2θ, and 27.3 °2θ (±0.2 °2θ; ±0.1 °2θ; or +0.0 °2θ; Cu Kα1 radiation).

In some embodiments, the R-ketamine sulfate salt is a crystalline polymorphic form characterized by PXRD peaks at 19.8 °2θ, 20.1 °2θ, 22.5 °2θ, 24.5 °2θ, 26.1 °2θ, and 27.3 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at two or more, or three or more peaks selected from the group consisting of 19.8 °2θ, 20.1 °2θ, 22.5 °2θ, 24.5 °2θ, 26.1 °2θ, 27.3 °2θ, and 30.4 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at 19.8 °2θ, 20.1 °2θ, 22.5 °2θ, 24.5 °2θ, 26.1 °2θ, 27.3 °2θ, and 30.4 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at two or more, or three or more peaks selected from the group consisting of 19.8 °2θ, 20.1 °2θ, 22.5 °2θ, 24.5 °2θ, 26.1 °2θ, 27.3 °2θ, 30.4 °2θ, and 30.9 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at 19.8 °2θ, 20.1 °2θ, 22.5 °2θ, 24.5 °2θ, 26.1 °2θ, 27.3 °2θ, 30.4 °2θ, and 30.9 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at two or more, or three or more peaks selected from the group consisting of 19.8 °2θ, 20.1 °2θ, 22.5 °2θ, 24.5 °2θ, 26.1 °2θ, 27.3 °2θ, 30.4 °2θ, 30.9 °2θ, and 32.7 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at 19.8 °2θ, 20.1 °2θ, 22.5 °2θ, 24.5 °2θ, 26.1 °2θ, 27.3 °2θ, 30.4 °2θ, 30.9 °2θ, and 32.7 °2θ (±0.2 °2θ; 0.1 °2θ; or ±0.0 °2θ; Cu Kα1 radiation).

In some embodiments, the R-ketamine sulfate salt is a crystalline polymorphic form characterized by PXRD peaks at two or more, or three or more peaks selected from the group consisting of 19.8 °2θ, 20.1 °2θ, 22.5 °2θ, 24.5 °2θ, 26.1 °2θ, 27.3 °2θ, 30.4 °2θ, 30.9 °2θ, 32.7 °2θ, and 35.5 °2θ (±0.2 °2θ; ±0.1 °2θ; or ±0.0 °2θ; Cu Kα1 radiation).

In some embodiments, the R-ketamine sulfate salt is a crystalline polymorphic form characterized by PXRD peaks at 19.8 °2θ, 20.1 °2θ, 22.5 °2θ, 24.5 °2θ, 26.1 °2θ, 27.3 °2θ, 30.4 °2θ, 30.9 °2θ, 32.7 °2θ, and 35.5 °2θ (±0.2 °2θ; ±0.1 °2θ; or ±0.0 °2θ; Cu Kα1 radiation).

In some embodiments, the R-ketamine sulfate salt is a crystalline polymorphic form characterized by one, two, three, four, five, six, seven, eight, nine, or ten PXRD peaks selected from those set forth in Table 6.

TABLE 6
R-ketamine Sulfate Salt PXRD (±0.2 °2θ; ±0.1 °2θ;
or ±0.0 °2θ; Cu Kα1 radiation).

| Angle in | d-spacing | | |
|---|---|---|---|
| °2-theta | in Å | Intensity | Intensity % |
| 19.8 | 4.48 | s | 37 |
| 20.1 | 4.41 | s | 34 |
| 22.5 | 3.95 | s | 35 |
| 24.5 | 3.63 | s | 32 |
| 26.1 | 3.41 | s | 35 |
| 27.3 | 3.26 | m | 27 |
| 30.4 | 2.94 | m | 22 |
| 30.9 | 2.89 | m | 22 |
| 32.7 | 2.74 | m | 18 |
| 35.5 | 2.53 | m | 17 |

### R-Ketamine D-Tartrate Salt Form A

In one aspect, the present disclosure provides an R-ketamine D-tartrate salt. In some embodiments, the R-ketamine D-tartrate salt is amorphous. In some embodiments, the R-ketamine D-tartrate salt is crystalline. In some embodiments, the R-ketamine D-tartrate salt is a crystalline polymorphic Form A. In some embodiments, the R-ketamine D-tartrate salt Form A can be characterized by the PXRD peaks set forth below in Table 7.

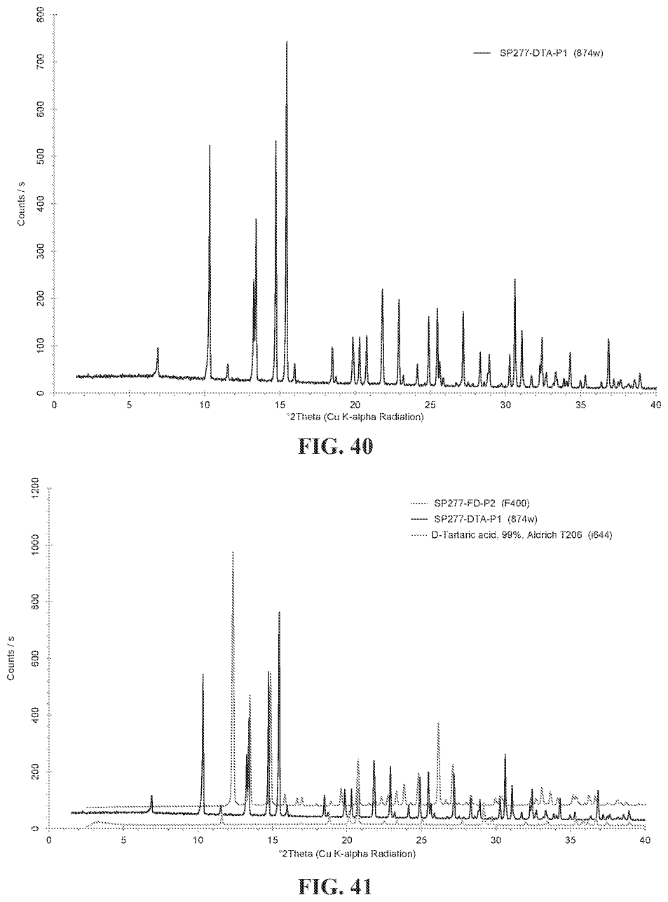

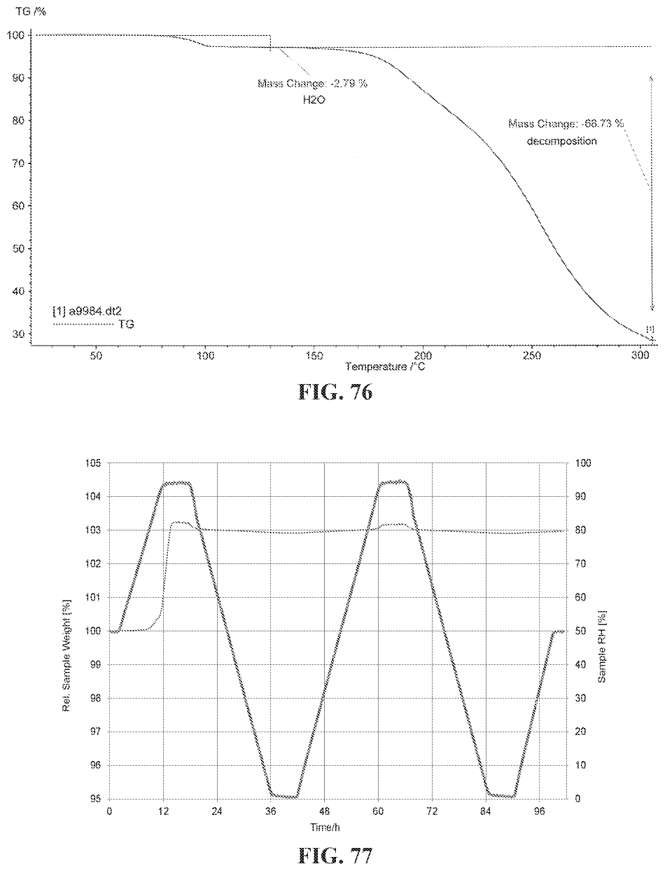

In some embodiments, the R-ketamine D-tartrate salt Form A PXRD spectrum is substantially similar to that shown in FIG. 40. In some embodiments, the R-ketamine D-tartrate salt Form A is a crystalline polymorphic form characterized by TG-FTIR substantially similar to that shown in FIG. 76. In some embodiments, the R-ketamine D-tartrate salt Form A is an ethanol solvate. In some embodiments, the R-ketamine D-tartrate salt Form A is a hydrate. In some embodiments, the R-ketamine D-tartrate salt Form A is an ethanol:water solvate. In some embodiments, the R-ketamine D-tartrate salt Form A is anhydrous.

In some embodiments, the R-ketamine D-tartrate salt Form A is a crystalline polymorphic form characterized by DSC substantially similar to that shown in FIG. 53. In some embodiments, the R-ketamine D-tartrate salt Form A is a crystalline polymorphic form characterized by a melting endotherm with a peak maximum at 108° C.

In some embodiments, the R-ketamine D-tartrate salt Form A is a 1:1 R-ketamine:D-tartaric acid salt. In some embodiments, the R-ketamine D-tartrate salt Form A is a 1:0.75 R-ketamine: D-tartaric acid salt. In some embodiments, the R-ketamine D-tartrate salt Form A is a 1:0.8 R-ketamine:D-tartaric acid salt. In some embodiments, the R-ketamine D-tartrate salt Form A is a 1:0.85 R-ketamine:D-tartaric acid salt. In some embodiments, the R-ketamine D-tartrate salt Form A is a 2:1 R-ketamine:D-tartaric acid salt. In some embodiments, the R-ketamine D-tartrate salt Form A is a 1:2 R-ketamine:D-tartaric acid salt.

In some embodiments, the R-ketamine D-tartrate salt is a crystalline polymorphic Form A characterized by a PXRD peak at 15.4 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 14.7 °2θ and 15.4 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 14.7 °2θ, and 15.4 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 14.7 °2θ, and 15.4 °2θ (±0.2 °2θ; ±0.1 °2θ; or 10.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.4 °2θ, 14.7 °2θ, and 15.4 °2θ (±0.2 °2θ; 0.1 °2θ; or 0.0 °2θ; Cu Kα1 radiation).

In some embodiments, the R-ketamine D-tartrate salt is a crystalline polymorphic Form A characterized by PXRD peaks at 10.3 °2θ, 13.4 °2θ, 14.7 °2θ, and 15.4 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.4 °2θ, 14.7 °2θ, 15.4 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.4 °2θ, 14.7 °2θ, 15.4 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.4 °2θ, 14.7 °2θ, 15.4 °2θ, 21.8 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.4 °2θ, 14.7 °2θ, 15.4 °2θ, 21.8 °2θ, and 30.62 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.4 °2θ, 14.7 °2θ, 15.4 °2θ, 21.8 °2θ, 22.9 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.4 °2θ, 14.7 °2θ, 15.4 °2θ, 21.8 °2θ, 22.9 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.4 °2θ, 14.7 °2θ, 15.4 °2θ, 21.8 °2θ, 22.9 °2θ, 25.5 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.4 °2θ, 14.7 °2θ, 15.4 °2θ, 21.8 °2θ, 22.9 °2θ, 25.5 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.2 °2θ, 13.4 °2θ, 14.7 °2θ, 15.4 °2θ, 21.8 °2θ, 22.9 °2θ, 25.5 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.2 °2θ, 13.4 °2θ, 14.7 °2θ, 15.4 °2θ, 21.8 °2θ, 22.9 °2θ, 25.5 °2θ, and 30.6 °2θ (±0.2 °2θ; ±0.1 °2θ; or 0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.2 °2θ, 13.4 °2θ, 14.7 °2θ, 15.4 °2θ, 21.8 °2θ, 22.9 °2θ, 25.5 °2θ, 27.2 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.2 °2θ, 13.4 °2θ, 14.7 °2θ, 15.4 °2θ, 21.8 °2θ, 22.9 °2θ, 25.5 °2θ, 27.2 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.2 °2θ, 13.4 °2θ, 14.7 °2θ, 15.4 °2θ, 21.8 °2θ, 22.9 °2θ, 24.9 °2θ, 25.5 °2θ, 27.2 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.2 °2θ, 13.4 °2θ, 14.7 °2θ, 15.4 °2θ, 21.8 °2θ, 22.9 °2θ, 24.9 °2θ, 25.5 °2θ, 27.2 °2θ, and 30.6 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.2 °2θ, 13.4 °2θ, 14.7 °2θ, 15.4 °2θ, 21.8 °2θ, 23.0 °2θ, 24.9 °2θ, 25.5 °2θ, 27.2 °2θ, 30.6 °2θ, and 31.1 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.2 °2θ, 13.4 °2θ, 14.7 °2θ, 15.4 °2θ, 21.8 °2θ, 22.9 °2θ, 24.9 °2θ, 25.5 °2θ, 27.2 °2θ, 30.6 °2θ, and 31.1 °2θ (L0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.2 °2θ, 13.4 °2θ, 14.7 °2θ, 15.4 °2θ, 19.9 °2θ, 21.8 °2θ, 22.9 °2θ, 24.9 °2θ, 25.5 °2θ, 27.2 °2θ, 30.6 °2θ, and 31.1 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.2 °2θ, 13.4 °2θ, 14.7 °2θ, 15.4 °2θ, 19.9 °2θ, 21.8 °2θ, 22.9 °2θ, 24.9 °2θ, 25.5 °2θ, 27.2 °2θ, 30.6 °2θ, and 31.1 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.2 °2θ, 13.4 °2θ, 14.7 °2θ, 15.4 °2θ, 19.9 °2θ, 20.3 °2θ, 21.8 °2θ, 22.9 °2θ, 24.9 °2θ, 25.5 °2θ, 27.2 °2θ, 30.6 °2θ, and 31.1 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.2 °2θ, 13.4 °2θ, 14.7 °2θ, 15.4 °2θ, 19.9 °2θ, 20.3 °2θ, 21.8 °2θ, 22.9 °2θ, 24.9 °2θ, 25.5 °2θ, 27.2 °2θ, 30.6 °2θ, and 31.1 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.2 °2θ, 13.4 °2θ, 14.7 °2θ, 15.4 °2θ, 19.9 °2θ, 20.3 °2θ, 20.8 °2θ, 21.8 °2θ, 22.9 °2θ, 24.9 °2θ, 25.5 °2θ, 27.2 °2θ, 30.6 °2θ, and 31.1 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.2 °2θ, 13.4 °2θ, 14.7 °2θ, 15.4 °2θ, 19.9 °2θ, 20.3 °2θ, 20.8 °2θ, 21.8 °2θ, 22.9 °2θ, 24.9 °2θ, 25.5 °2θ, 27.2 °2θ, 30.6 °2θ, and 31.1 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.2 °2θ, 13.4 °2θ, 14.7 °2θ, 15.4 °2θ, 19.9 °2θ, 20.3 °2θ, 20.8 °2θ, 21.8 °2θ, 22.9 °2θ, 24.9 °2θ, 25.5 °2θ, 27.2 °2θ, 30.6 °2θ, 31.1 °2θ, and 32.4 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.2 °2θ, 13.4 °2θ, 14.7 °2θ, 15.4 °2θ, 19.9 °2θ, 20.3 °2θ, 20.8 °2θ, 21.8 °2θ, 22.9 °2θ, 24.9 °2θ, 25.5 °2θ, 27.2 °2θ, 30.6 °2θ, 31.1 °2θ, and 32.4 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 10.3 °2θ, 13.2 °2θ, 13.4 °2θ, 14.7 °2θ, 15.4 °2θ, 19.9 °2θ, 20.3 °2θ, 20.8 °2θ, 21.8 °2θ, 22.9 °2θ, 24.9 °2θ, 25.5 °2θ, 27.2 °2θ, 30.6 °2θ, 31.1 °2θ, 32.4 °2θ, and 36.9 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 10.3 °2θ, 13.2 °2θ, 13.4 °2θ, 14.7 °2θ, 15.4 °2θ, 19.9 °2θ, 20.3 °2θ, 20.8 °2θ, 21.8 °2θ, 22.9 °2θ, 24.9 °2θ, 25.5 °2θ, 27.2 °2θ, 30.6 °2θ, 31.1 °2θ, 32.4 °2θ, and 36.9 °2θ (±0.2 °2θ; 0.1 °2θ; or ±0.0 °2θ; Cu Kα1 radiation).

In some embodiments, the R-ketamine D-tartrate salt is a crystalline polymorphic Form A characterized by PXRD peaks at two or more, or three or more peaks selected from the group consisting of 6.9 °2θ, 10.3 °2θ, 13.2 °2θ, 13.4 °2θ, 14.7 °2θ, 15.4 °2θ, 19.9 °2θ, 20.3 °2θ, 20.8 °2θ, 21.8 °2θ, 22.9 °2θ, 24.9 °2θ, 25.5 °2θ, 27.2 °2θ, 30.6 °2θ, 31.1 °2θ, 32.4 °2θ, and 36.9 °2θ (±0.2 °2θ; ±0.1 °2θ; or ±0.0 °2θ; Cu Kα1 radiation).

In some embodiments, the R-ketamine D-tartrate salt is a crystalline polymorphic Form A characterized by PXRD peaks at 6.9 °2θ, 10.3 °2θ, 13.2 °2θ, 13.4 °2θ, 14.7 °2θ, 15.4 °2θ, 19.9 °2θ, 20.3 °2θ, 20.8 °2θ, 21.8 °2θ, 22.9 °2θ, 24.9 °2θ, 25.5 °2θ, 27.2 °2θ, 30.6 °2θ, 31.1 °2θ, 32.4 °2θ, and 36.9 °2θ (±0.2 °2θ; ±0.1 °2θ; or ±0.0 °2θ; Cu Kα1 radiation).

In some embodiments, the R-ketamine D-tartrate salt Form A is characterized by one, two, three, four, five, six, seven, eight, nine, ten, eleven, twelve, thirteen, fourteen, fifteen, or sixteen PXRD peaks selected from those set forth in Table 7.

TABLE 7
R-ketamine D-tartrate Salt Form A PXRD (±0.2 °2θ; ±0.1 °2θ;
or ±0.0 °2θ; Cu Kα1 radiation).

| Angle in | d-spacing | | |
|---|---|---|---|
| °2-theta | in Å | Intensity | Intensity % |
| 6.9 | 12.86 | w | 13 |
| 10.3 | 8.56 | vs | 71 |
| 11.5 | 7.66 | w | 8 |
| 13.2 | 6.69 | m | 23 |
| 13.4 | 6.61 | s | 41 |
| 14.7 | 6.01 | vs | 72 |
| 15.4 | 5.73 | vs | 100 |
| 16.0 | 5.55 | w | 9 |
| 18.5 | 4.80 | w | 13 |
| 18.7 | 4.73 | vw | 5 |
| 19.9 | 4.47 | m | 16 |
| 20.3 | 4.37 | m | 16 |
| 20.8 | 4.27 | m | 16 |
| 21.8 | 4.07 | m | 30 |
| 22.9 | 3.88 | m | 27 |
| 23.2 | 3.83 | w | 5 |
| 24.1 | 3.68 | w | 8 |
| 24.9 | 3.58 | m | 22 |
| 25.5 | 3.49 | m | 24 |
| 25.9 | 3.44 | vw | 5 |
| 26.7 | 3.33 | vw | 3 |
| 27.2 | 3.28 | m | 23 |
| 27.5 | 3.24 | vw | 3 |
| 27.8 | 3.20 | vw | 3 |
| 28.3 | 3.15 | w | 12 |
| 28.6 | 3.12 | vw | 3 |
| 28.9 | 3.09 | w | 11 |
| 29.7 | 3.00 | vw | 3 |
| 30.3 | 2.95 | w | 11 |
| 30.6 | 2.92 | s | 32 |
| 31.1 | 2.87 | m | 18 |
| 31.7 | 2.82 | w | 5 |
| 32.4 | 2.76 | m | 16 |
| 32.7 | 2.74 | w | 6 |
| 33.4 | 2.68 | w | 6 |
| 33.9 | 2.65 | vw | 4 |
| 34.3 | 2.61 | w | 12 |
| 35.0 | 2.56 | vw | 4 |
| 35.3 | 2.54 | w | 5 |
| 36.4 | 2.47 | vw | 3 |
| 36.9 | 2.44 | m | 15 |
| 37.2 | 2.42 | vw | 4 |
| 37.6 | 2.39 | vw | 4 |
| 38.2 | 2.36 | vw | 3 |
| 38.6 | 2.33 | vw | 4 |
| 38.9 | 2.31 | w | 6 |
| 40.3 | 2.24 | w | 5 |
| 41.0 | 2.20 | vw | 5 |

### R-Ketamine D-Tartrate Salt Form B

In one aspect, the present disclosure provides an R-ketamine D-tartrate salt. In some embodiments, the R-ketamine D-tartrate salt is amorphous. In some embodiments, the R-ketamine D-tartrate salt is crystalline. In some embodiments, the R-ketamine D-tartrate salt is a crystalline polymorphic Form B. In some embodiments, the R-ketamine D-tartrate salt Form B can be characterized by the PXRD peaks set forth below in Table 8.

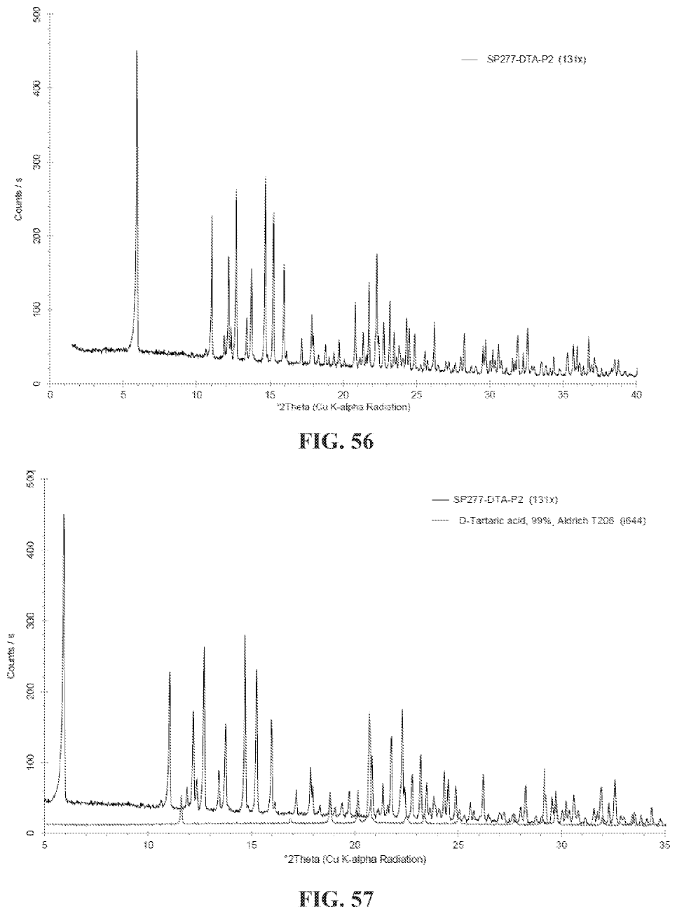

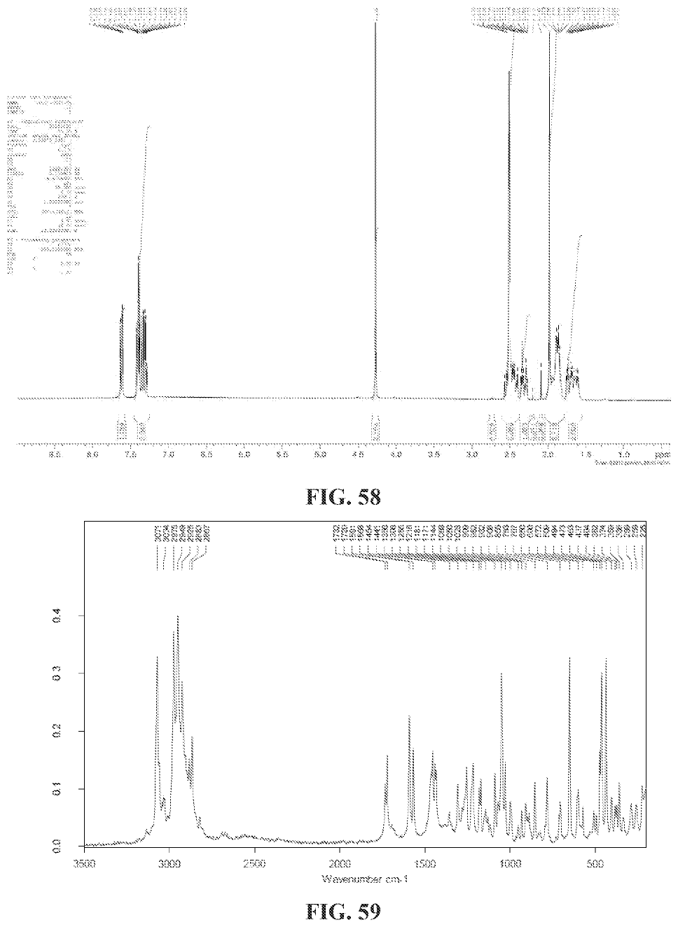

In some embodiments, the R-ketamine D-tartrate salt Form B is a crystalline polymorphic form characterized by a PXRD spectrum substantially similar to that shown in FIG. 57. In some embodiments, the R-ketamine D-tartrate salt Form B is a crystalline polymorphic form characterized by a FT-Raman spectrum substantially similar to that shown in FIG. 59. In some embodiments, the R-ketamine D-tartrate salt Form B is an acetone solvate. In some embodiments, the R-ketamine D-tartrate salt Form B is a hydrate. In some embodiments, the R-ketamine D-tartrate salt Form B is an acetone:water solvate. In some embodiments, the R-ketamine D-tartrate salt Form B is anhydrous. In some embodiments, the R-ketamine D-tartrate salt Form B is a crystalline polymorphic form characterized by a ¹H-NMR according to FIG. 58.

In some embodiments, the R-ketamine D-tartrate salt Form B is a 1:1 R-ketamine:D-tartaric acid salt. In some embodiments, the R-ketamine D-tartrate salt Form B is a 1:0.75 R-ketamine:D-tartaric acid salt. In some embodiments, the R-ketamine D-tartrate salt Form B is a 1:0.8 R-ketamine:D-tartaric acid salt. In some embodiments, the R-ketamine D-tartrate salt Form B is a 1:0.85 R-ketamine:D-tartaric acid salt. In some embodiments, the R-ketamine D-tartrate salt Form B is a 2:1 R-ketamine:D-tartaric acid salt. In some embodiments, the R-ketamine D-tartrate salt Form B is a 1:2 R-ketamine:D-tartaric acid salt.

In some embodiments, the R-ketamine D-tartrate salt is a crystalline polymorphic Form B characterized by a PXRD peak at 5.9 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ and 14.7 °2θ (±0.2 °2θ; ±0.1 °2θ; or 10.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 12.7 °2θ, and 14.7 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 12.7 °2θ, and 14.7 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.7 °2θ, and 14.7 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.7 °2θ, and 14.7 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.7 °2θ, and 14.7 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.7 °2θ, and 14.7 °2θ (±0.2 °2θ; ±0.1 °2θ; or +0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.7 °2θ, 14.7 °2θ, and 15.2 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.7 °2θ, 14.7 °2θ, and 15.2 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.7 °2θ, 14.7 °2θ, 15.2 °2θ, and 22.3 °2θ (±0.2 °2θ; ±0.1 °2θ; or +0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.7 °2θ, 14.7 °2θ, 15.2 °2θ, and 22.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.2 °2θ, 12.7 °2θ, 14.7 °2θ, 15.2 °2θ, and 22.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.2 °2θ, 12.7 °2θ, 14.7 °2θ, 15.2 °2θ, and 22.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.2 °2θ, 12.7 °2θ, 14.7 °2θ, 15.2 °2θ, 16.0 °2θ, and 22.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.2 °2θ, 12.7 °2θ, 14.7 °2θ, 15.2 °2θ, 16.0 °2θ, and 22.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.2 °2θ, 12.7 °2θ, 13.7 °2θ, 14.7 °2θ, 15.2 °2θ, 16.0 °2θ, and 22.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.2 °2θ, 12.7 °2θ, 13.7 °2θ, 14.7 °2θ, 15.2 °2θ, 16.0 °2θ, and 22.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.2 °2θ, 12.7 °2θ, 13.7 °2θ, 14.7 °2θ, 15.2 °2θ, 16.0 °2θ, 21.8 °2θ, and 22.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.2 °2θ, 12.7 °2θ, 13.7 °2θ, 14.7 °2θ, 15.2 °2θ, 16.0 °2θ, 21.8 °2θ, and 22.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.2 °2θ, 12.7 °2θ, 13.7 °2θ, 14.7 °2θ, 15.2 °2θ, 16.0 °2θ, 20.8 °2θ, 21.8 °2θ, and 22.3 °2θ (±0.2 °2θ; ±0.1 °2θ; or 0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.2 °2θ, 12.7 °2θ, 13.7 °2θ, 14.7 °2θ, 15.2 °2θ, 16.0 °2θ, 20.8 °2θ, 21.8 °2θ, and 22.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.2 °2θ, 12.7 °2θ, 13.7 °2θ, 14.7 °2θ, 15.2 °2θ, 16.0 °2θ, 20.8 °2θ, 21.8 °2θ, 22.3 °2θ, and 23.2 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.2 °2θ, 12.7 °2θ, 13.7 °2θ, 14.7 °2θ, 15.2 °2θ, 16.0 °2θ, 20.8 °2θ, 21.8 °2θ, 22.3 °2θ, and 23.2 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.2 °2θ, 12.7 °2θ, 13.7 °2θ, 14.7 °2θ, 15.2 °2θ, 16.0 °2θ, 17.9 °2θ, 20.8 °2θ, 21.8 °2θ, 22.3 °2θ, and 23.2 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.2 °2θ, 12.7 °2θ, 13.7 °2θ, 14.7 °2θ, 15.2 °2θ, 16.0 °2θ, 17.9 °2θ, 20.8 °2θ, 21.8 °2θ, 22.3 °2θ, and 23.2 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.2 °2θ, 12.7 °2θ, 13.4 °2θ, 13.7 °2θ, 14.7 °2θ, 15.2 °2θ, 16.0 °2θ, 17.9 °2θ, 20.8 °2θ, 21.8 °2θ, 22.3 °2θ, and 23.2 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.2 °2θ, 12.7 °2θ, 13.4 °2θ, 13.7 °2θ, 14.7 °2θ, 15.2 °2θ, 16.0 °2θ, 17.9 °2θ, 20.8 °2θ, 21.8 °2θ, 22.3 °2θ, and 23.2 °2θ (L0.2 °2θ; ±0.1 °2θ; or +0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.2 °2θ, 12.7 °2θ, 13.4 °2θ, 13.7 °2θ, 14.7 °2θ, 15.2 °2θ, 16.0 °2θ, 17.9 °2θ, 20.8 °2θ, 21.8 °2θ, 22.3 °2θ, 23.2 °2θ, and 24.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.2 °2θ, 12.7 °2θ, 13.4 °2θ, 13.7 °2θ, 14.7 °2θ, 15.2 °2θ, 16.0 °2θ, 17.9 °2θ, 20.8 °2θ, 21.8 °2θ, 22.3 °2θ, 23.2 °2θ, and 24.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.2 °2θ, 12.7 °2θ, 13.4 °2θ, 13.7 °2θ, 14.7 °2θ, 15.2 °2θ, 16.0 °2θ, 17.9 °2θ, 20.8 °2θ, 21.8 °2θ, 22.3 °2θ, 22.8 °2θ, 23.2 °2θ, and 24.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.2 °2θ, 12.7 °2θ, 13.4 °2θ, 13.7 °2θ, 14.7 °2θ, 15.2 °2θ, 16.0 °2θ, 17.9 °2θ, 20.8 °2θ, 21.8 °2θ, 22.3 °2θ, 22.8 °2θ, 23.2 °2θ, and 24.3 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at two or more, or three or more peaks selected from the group consisting of 5.9 °2θ, 11.0 °2θ, 12.2 °2θ, 12.7 °2θ, 13.4 °2θ, 13.7 °2θ, 14.7 °2θ, 15.2 °2θ, 16.0 °2θ, 17.9 °2θ, 20.8 °2θ, 21.8 °2θ, 22.3 °2θ, 22.8 °2θ, 23.2 °2θ, 24.3 °2θ, and 26.2 °2θ (±0.2 °2θ; ±0.1 °2θ; or ±0.0 °2θ; Cu Kα1 radiation).

In some embodiments, the R-ketamine D-tartrate salt is a crystalline polymorphic Form B characterized by PXRD peaks at 5.9 °2θ, 11.0 °2θ, 12.2 °2θ, 12.7 °2θ, 13.4 °2θ, 13.7 °2θ, 14.7 °2θ, 15.2 °2θ, 16.0 °2θ, 17.9 °2θ, 20.8 °2θ, 21.8 °2θ, 22.3 °2θ, 22.8 °2θ, 23.2 °2θ, 24.3 °2θ, and 26.2 °2θ (±0.2 °2θ; ±0.1 °2θ; or ±0.0 °2θ; Cu Kα1 radiation).

In some embodiments, the R-ketamine D-tartrate salt Form B form is characterized by one, two, three, four, five, six, seven, eight, nine, ten, eleven, twelve, thirteen, fourteen, fifteen, or sixteen PXRD peaks selected from those set forth in Table 8.

TABLE 8
R-ketamine D-tartrate Salt Form B PXRD (±0.2 °2θ; ±0.1 °2θ;
or ±0.0 °2θ; Cu Kα1 radiation).

| Angle in | d-spacing | | |
|---|---|---|---|
| °2-theta | in Å | Intensity | Intensity % |
| 5.9 | 14.93 | vs | 100 |
| 11.0 | 8.01 | s | 51 |
| 11.9 | 7.45 | w | 15 |
| 12.2 | 7.26 | s | 38 |
| 12.7 | 6.97 | s | 58 |
| 13.4 | 6.59 | m | 20 |
| 13.7 | 6.44 | s | 34 |
| 14.7 | 6.03 | s | 62 |
| 15.2 | 5.81 | s | 51 |
| 16.0 | 5.55 | s | 36 |
| 17.2 | 5.16 | w | 14 |
| 17.9 | 4.96 | m | 21 |
| 18.3 | 4.84 | w | 9 |
| 18.8 | 4.72 | w | 12 |
| 19.4 | 4.58 | w | 10 |
| 19.7 | 4.50 | w | 13 |
| 20.1 | 4.42 | w | 7 |
| 20.8 | 4.26 | m | 25 |
| 21.4 | 4.16 | m | 16 |
| 21.8 | 4.08 | s | 30 |
| 22.3 | 3.98 | s | 39 |
| 22.8 | 3.90 | m | 19 |
| 23.2 | 3.83 | m | 25 |
| 23.5 | 3.78 | m | 16 |
| 23.9 | 3.73 | w | 11 |
| 24.3 | 3.65 | m | 20 |
| 24.6 | 3.62 | w | 15 |
| 24.9 | 3.57 | w | 15 |
| 25.6 | 3.48 | w | 10 |
| 26.2 | 3.40 | m | 19 |
| 26.5 | 3.36 | w | 6 |
| 27.2 | 3.28 | w | 6 |
| 27.6 | 3.23 | w | 6 |
| 28.0 | 3.18 | w | 8 |
| 28.3 | 3.15 | m | 15 |
| 28.8 | 3.10 | w | 6 |
| 29.1 | 3.07 | w | 5 |
| 29.7 | 3.01 | w | 11 |
| 30.3 | 2.95 | w | 7 |
| 30.6 | 2.92 | w | 12 |
| 31.2 | 2.87 | vw | 5 |
| 31.6 | 2.83 | w | 7 |
| 31.9 | 2.80 | w | 15 |
| 32.3 | 2.77 | w | 9 |
| 32.6 | 2.74 | m | 17 |
| 33.0 | 2.72 | vw | 5 |
| 33.5 | 2.67 | w | 7 |
| 33.8 | 2.65 | w | 6 |
| 34.4 | 2.61 | w | 8 |
| 34.8 | 2.58 | vw | 5 |
| 35.3 | 2.54 | w | 9 |
| 35.7 | 2.51 | w | 12 |
| 36.0 | 2.49 | w | 11 |
| 36.4 | 2.47 | w | 5 |
| 36.8 | 2.44 | w | 14 |
| 37.2 | 2.42 | w | 8 |
| 37.7 | 2.39 | vw | 5 |
| 38.5 | 2.33 | w | 7 |
| 38.8 | 2.32 | w | 7 |
| 39.2 | 2.29 | vw | 4 |
| 39.6 | 2.27 | vw | 3 |
| 40.1 | 2.25 | vw | 5 |
| 40.4 | 2.23 | vw | 4 |

### R-Ketamine D-Tartrate Salt Form C

In one aspect, the present disclosure provides an R-ketamine D-tartrate salt. In some embodiments, the R-ketamine D-tartrate salt is amorphous. In some embodiments, the R-ketamine D-tartrate salt is crystalline. In some embodiments, the R-ketamine D-tartrate salt is a crystalline polymorphic Form C. In some embodiments, the R-ketamine D-tartrate salt Form C can be characterized by the PXRD peaks set forth below in Table 9.

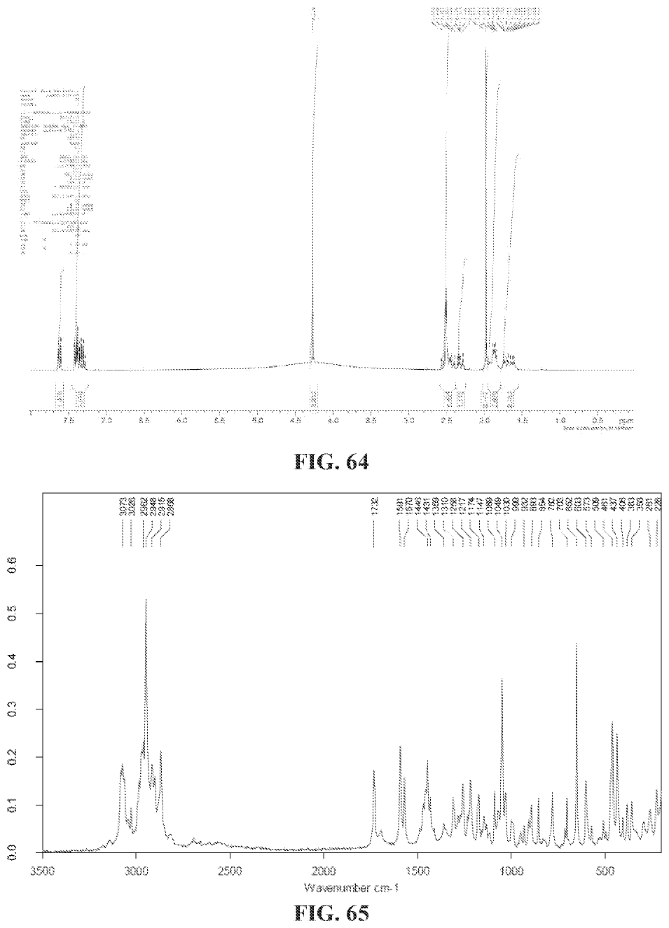

In some embodiments, the R-ketamine D-tartrate salt Form C is a crystalline polymorphic form characterized by a PXRD spectrum substantially similar to that shown in FIG. 61. In some embodiments, the R-ketamine D-tartrate salt Form C is a crystalline polymorphic form characterized by an FT-Raman spectrum substantially similar to that shown in FIG. 65. In some embodiments, the R-ketamine D-tartrate salt Form C is an ethyl acetate solvate. In some embodiments, the R-ketamine D-tartrate salt Form C is an methanol solvate. In some embodiments, the R-ketamine D-tartrate salt Form C is a hydrate. In some embodiments, the R-ketamine D-tartrate salt Form C is a methanol:ethyl acetate solvate. In some embodiments, the R-ketamine D-tartrate salt Form C is anhydrous. In some embodiments, the R-ketamine D-tartrate salt Form C is a crystalline polymorphic form characterized by a ¹H-NMR according to FIG. 64.

In some embodiments, the R-ketamine D-tartrate salt Form C is a 1:1 R-ketamine:D-tartaric acid salt. In some embodiments, the R-ketamine D-tartrate salt Form C is a 1:1.25 R-ketamine:D-tartaric acid salt. In some embodiments, the R-ketamine D-tartrate salt Form C is a 1:1.5 R-ketamine:D-tartaric acid salt. In some embodiments, the R-ketamine D-tartrate salt Form C is a 1:0.85 R-ketamine:D-tartaric acid salt. In some embodiments, the R-ketamine D-tartrate salt Form C is a 2:1 R-ketamine:D-tartaric acid salt. In some embodiments, the R-ketamine D-tartrate salt Form C is a 1:2 R-ketamine:D-tartaric acid salt. In some embodiments, the R-ketamine D-tartrate salt Form C is a 1:1.15 R-ketamine:D-tartaric acid salt.

In some embodiments, the R-ketamine D-tartrate salt is a crystalline polymorphic Form C characterized by a PXRD peak at 6.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by a PXRD peak at 14.0 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ and 11.0 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 11.0 °2θ and 14.0 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, and 12.2 °2θ (±0.2 °2θ; 0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 11.0 °2θ, 13.6 °2θ, and 14.0 °2θ (±0.2 °2θ; 0.1 °2θ; or 0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, and 12.2 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 11.0 °2θ, 13.6 °2θ, and 14.0 °2θ (±0.2 °2θ; 0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, and 14.0 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 13.6 °2θ, and 14.0 °2θ (±0.2 °2θ; ±0.1 °2θ; or ±0.0 °2θ; Cu Kα1 radiation). In some embodiments, the R-ketamine D-tartrate salt is a crystalline polymorphic Form C characterized by PXRD peaks at 6.1 °2θ, 11.0 °2θ, 12.2 °2θ, and 14.0 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 13.6 °2θ, and 14.0 °2θ (±0.2 °2θ; ±0.1 °2θ; or +0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.5 °2θ, and 14.0 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.5 °2θ, and 14.0 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.5 °2θ, 14.0 °2θ, and 21.3 °2θ (±0.2 °2θ; ±0.1 °2θ; or +0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.5 °2θ, 14.0 °2θ, and 21.3 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.5 °2θ, 14.0 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.5 °2θ, 15.6 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.5 °2θ, 14.0 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.5 °2θ, 15.6 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.5 °2θ, 14.0 °2θ, 15.6 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.5 °2θ, 14.6 °2θ, 15.6 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.5 °2θ, 14.0 °2θ, 15.6 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.5 °2θ, 14.6 °2θ, 15.6 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.5 °2θ, 14.0 °2θ, 15.6 °2θ, 18.4 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.0 °2θ, 13.5 °2θ, 14.6 °2θ, 15.6 °2θ, 18.4 °2θ, and 21.3 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.5 °2θ, 14.0 °2θ, 15.6 °2θ, 18.4 °2θ, 21.3 °2θ, and 24.1 °2θ (±0.2 °2θ; ±0.1 °2θ; or 0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.0 °2θ, 13.5 °2θ, 14.6 °2θ, 15.6 °2θ, 18.4 °2θ, and 21.3 °2θ (±0.2 °2θ; ±0.1 °2θ; or 0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.5 °2θ, 14.0 °2θ, 14.6 °2θ, 15.6 °2θ, 18.4 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.0 °2θ, 13.5 °2θ, 14.6 °2θ, 15.6 °2θ, 18.4 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.5 °2θ, 14.0 °2θ, 14.6 °2θ, 15.6 °2θ, 18.4 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.0 °2θ, 13.5 °2θ, 14.6 °2θ, 15.6 °2θ, 18.4 °2θ, 21.3 °2θ, and 24.1 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.5 °2θ, 14.0 °2θ, 14.6 °2θ, 15.6 °2θ, 18.4 °2θ, 21.3 °2θ, 24.1 °2θ, and 24.5 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 13.0 °2θ, 13.5 °2θ, 14.0 °2θ, 14.6 °2θ, 15.6 °2θ, 18.4 °2θ, 21.3 °2θ, and 24.1 °2θ (±0.2 °2θ; ±0.1 °2θ; or 0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.5 °2θ, 14.0 °2θ, 14.6 °2θ, 15.6 °2θ, 18.4 °2θ, 21.3 °2θ, 24.1 °2θ, and 24.5 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 13.0 °2θ, 13.5 °2θ, 14.0 °2θ, 14.6 °2θ, 15.6 °2θ, 18.4 °2θ, 21.3 °2θ, and 24.1 °2θ (±0.2 °2θ; 0.1 °2θ; or 0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 12.9 °2θ, 13.5 °2θ, 14.0 °2θ, 14.6 °2θ, 15.6 °2θ, 18.4 °2θ, 21.3 °2θ, 24.1 °2θ, and 24.5 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 12.9 °2θ, 13.5 °2θ, 14.0 °2θ, 14.6 °2θ, 15.6 °2θ, 18.4 °2θ, 21.3 °2θ, 24.1 °2θ, and 24.5 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 12.9 °2θ, 13.5 °2θ, 14.0 °2θ, 14.6 °2θ, 15.6 °2θ, 18.4 °2θ, 21.3 °2θ, 24.1 °2θ, and 24.5 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 12.9 °2θ, 13.5 °2θ, 14.0 °2θ, 14.6 °2θ, 15.6 °2θ, 17.6 °2θ, 18.4 °2θ, 21.3 °2θ, 24.1 °2θ, and 24.5 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 12.9 °2θ, 13.5 °2θ, 14.0 °2θ, 14.6 °2θ, 15.6 °2θ, 17.6 °2θ, 18.4 °2θ, 21.3 °2θ, 24.1 °2θ, and 24.5 (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 12.9 °2θ, 13.5 °2θ, 14.0 °2θ, 14.6 °2θ, 15.6 °2θ, 16.1 °2θ, 17.6 °2θ, 18.4 °2θ, 21.3 °2θ, 24.1 °2θ, and 24.5 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 12.9 °2θ, 13.5 °2θ, 14.0 °2θ, 14.6 °2θ, 15.6 °2θ, 16.1 °2θ, 17.6 °2θ, 18.4 °2θ, 21.3 °2θ, 24.1 °2θ, and 24.5 °2θ (±0.2 °2θ; ±0.1 °2θ; or +0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 12.9 °2θ, 13.5 °2θ, 14.0 °2θ, 14.6 °2θ, 15.6 °2θ, 16.1 °2θ, 17.6 °2θ, 18.4 °2θ, 21.3 °2θ, 22.1 °2θ, 24.1 °2θ, and 24.5 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 12.9 °2θ, 13.5 °2θ, 14.0 °2θ, 14.6 °2θ, 15.6 °2θ, 16.1 °2θ, 17.6 °2θ, 18.4 °2θ, 21.3 °2θ, 22.1 °2θ, 24.1 °2θ, and 24.5 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at two or more, or three or more peaks selected from the group consisting of 6.1 °2θ, 11.0 °2θ, 12.2 °2θ, 12.9 °2θ, 13.5 °2θ, 14.0 °2θ, 14.6 °2θ, 15.6 °2θ, 16.1 °2θ, 17.6 °2θ, 18.4 °2θ, 21.3 °2θ, 22.1 °2θ, 23.1 °2θ, 24.1 °2θ, and 24.5 °2θ (±0.2 °2θ; ±0.1 °2θ; or ±0.0 °2θ; Cu Kα1 radiation).

In some embodiments, the R-ketamine D-tartrate salt is a crystalline polymorphic Form C characterized by PXRD peaks at 6.1 °2θ, 11.0 °2θ, 12.2 °2θ, 12.9 °2θ, 13.5 °2θ, 14.0 °2θ, 14.6 °2θ, 15.6 °2θ, 16.1 °2θ, 17.6 °2θ, 18.4 °2θ, 21.3 °2θ, 22.1 °2θ, 23.1 °2θ, 24.1 °2θ, and 24.5 °2θ (±0.2 °2θ; ±0.1 °2θ; or ±0.0 °2θ; Cu Kα1 radiation).

In some embodiments, the R-ketamine D-tartrate salt Form C is characterized by one, two, three, four, five, six, seven, eight, nine, ten, eleven, twelve, thirteen, fourteen, fifteen, or sixteen PXRD peaks selected from those set forth in Table 9.

In some embodiments, the R-ketamine D-tartrate salt Form C is characterized by one, two, three, four, five, six, seven, eight, nine, ten, eleven, twelve, thirteen, fourteen, fifteen, or sixteen PXRD peaks selected from those set forth in Table 10.

TABLE 9
R-ketamine D-tartrate Salt Form C PXRD (±0.2 °2θ; ±0.1 °2θ;
or ±0.0 °2θ; Cu Kα1 radiation).

| Angle in | d-spacing | | |
|---|---|---|---|
| °2-theta | in Å | Intensity | Intensity % |
| 6.1 | 14.49 | vs | 100 |
| 11.0 | 8.06 | s | 67 |
| 12.2 | 7.25 | s | 69 |
| 12.6 | 7.04 | m | 19 |
| 12.9 | 6.84 | s | 34 |
| 13.5 | 6.53 | s | 56 |
| 14.0 | 6.32 | vs | 93 |
| 14.6 | 6.08 | s | 44 |
| 14.8 | 5.97 | m | 23 |
| 15.6 | 5.68 | s | 54 |
| 16.1 | 5.49 | m | 30 |
| 17.6 | 5.04 | s | 32 |
| 18.0 | 4.91 | m | 27 |
| 18.4 | 4.81 | s | 51 |
| 18.7 | 4.75 | m | 27 |
| 19.9 | 4.46 | w | 14 |
| 20.3 | 4.37 | m | 20 |
| 20.6 | 4.30 | m | 19 |
| 21.3 | 4.17 | s | 55 |
| 22.1 | 4.03 | m | 29 |
| 22.4 | 3.97 | w | 15 |
| 22.7 | 3.91 | m | 16 |
| 23.1 | 3.84 | m | 29 |
| 23.6 | 3.76 | m | 15 |
| 23.8 | 3.74 | m | 16 |
| 24.1 | 3.70 | s | 55 |
| 24.5 | 3.62 | s | 36 |
| 25.0 | 3.55 | m | 24 |
| 25.4 | 3.51 | m | 21 |
| 26.1 | 3.41 | w | 13 |
| 26.7 | 3.33 | m | 19 |
| 27.0 | 3.30 | w | 10 |
| 27.8 | 3.21 | m | 18 |
| 28.1 | 3.17 | m | 18 |
| 28.6 | 3.12 | w | 11 |
| 29.1 | 3.07 | w | 12 |
| 29.4 | 3.04 | w | 8 |
| 29.9 | 2.98 | m | 18 |
| 30.9 | 2.89 | m | 17 |
| 31.5 | 2.84 | w | 11 |
| 32.2 | 2.78 | m | 25 |
| 32.6 | 2.75 | w | 13 |
| 33.6 | 2.67 | w | 14 |
| 34.0 | 2.63 | w | 9 |
| 34.5 | 2.60 | w | 11 |
| 35.3 | 2.54 | w | 14 |
| 36.0 | 2.50 | w | 9 |
| 36.6 | 2.45 | w | 12 |
| 37.1 | 2.42 | m | 15 |
| 38.0 | 2.36 | w | 11 |
| 38.9 | 2.31 | w | 9 |
| 39.8 | 2.26 | w | 8 |
| 40.4 | 2.23 | w | 5 |
| 40.8 | 2.21 | w | 8 |

TABLE 10
R-ketamine D-tartrate Salt Form C PXRD (±0.2 °2θ; ±0.1 °2θ;
or ±0.0 °2θ; Cu Kα1 radiation).

| Angle in | d-spacing | | |
|---|---|---|---|
| °2-theta | in Å | Intensity | Intensity % |
| 6.1 | 14.45 | s | 63 |
| 11.0 | 8.05 | s | 69 |
| 12.2 | 7.24 | s | 63 |
| 12.6 | 7.03 | m | 21 |
| 13.0 | 6.83 | s | 41 |
| 13.6 | 6.52 | s | 65 |
| 14.0 | 6.32 | vs | 100 |
| 14.6 | 6.07 | s | 49 |
| 15.6 | 5.67 | s | 53 |
| 16.1 | 5.49 | m | 28 |
| 17.6 | 5.03 | m | 29 |
| 18.1 | 4.91 | m | 24 |
| 18.5 | 4.8 | s | 37 |
| 18.7 | 4.75 | m | 23 |
| 19.4 | 4.57 | w | 13 |
| 19.9 | 4.46 | w | 15 |
| 20.1 | 4.41 | m | 22 |
| 20.4 | 4.36 | m | 18 |
| 20.6 | 4.3 | m | 19 |
| 21.3 | 4.17 | s | 54 |
| 22.1 | 4.02 | s | 32 |
| 22.4 | 3.96 | m | 16 |
| 22.8 | 3.9 | w | 14 |
| 23.1 | 3.84 | m | 28 |
| 23.7 | 3.76 | m | 20 |
| 23.9 | 3.73 | m | 20 |
| 24.0 | 3.7 | s | 41 |
| 24.6 | 3.62 | s | 33 |
| 25.0 | 3.56 | m | 19 |
| 25.4 | 3.51 | m | 17 |
| 26.1 | 3.42 | w | 12 |
| 26.3 | 3.39 | w | 13 |
| 26.7 | 3.33 | m | 17 |
| 27.0 | 3.3 | w | 10 |
| 27.8 | 3.21 | w | 14 |
| 28.1 | 3.17 | m | 15 |
| 28.6 | 3.12 | w | 12 |
| 29.1 | 3.07 | w | 13 |
| 29.4 | 3.04 | w | 8 |
| 29.9 | 2.98 | m | 17 |
| 30.9 | 2.89 | m | 18 |
| 31.0 | 2.88 | m | 17 |
| 31.5 | 2.84 | w | 10 |
| 32.2 | 2.78 | m | 23 |
| 32.6 | 2.74 | w | 12 |
| 33.4 | 2.68 | w | 13 |
| 33.5 | 2.67 | w | 12 |
| 33.8 | 2.65 | w | 9 |
| 34.0 | 2.63 | w | 8 |
| 34.5 | 2.6 | w | 10 |
| 34.7 | 2.58 | w | 7 |
| 35.3 | 2.54 | w | 14 |
| 36.0 | 2.49 | w | 11 |
| 36.6 | 2.45 | w | 14 |
| 37.1 | 2.42 | m | 16 |
| 38.0 | 2.36 | w | 11 |
| 38.5 | 2.33 | w | 6 |
| 38.9 | 2.31 | w | 10 |
| 39.8 | 2.26 | w | 8 |
| 40.4 | 2.23 | w | 6 |
| 40.8 | 2.21 | w | 8 |

### R-Ketamine D-Tartrate Salt Form D

In one aspect, the present disclosure provides an R-ketamine D-tartrate salt. In some embodiments, the R-ketamine D-tartrate salt is amorphous. In some embodiments, the R-ketamine D-tartrate salt is crystalline. In some embodiments, the R-ketamine D-tartrate salt is a crystalline polymorphic Form D. In some embodiments, the R-ketamine D-tartrate salt Form D can be characterized by the PXRD peaks set forth below in Table 11.

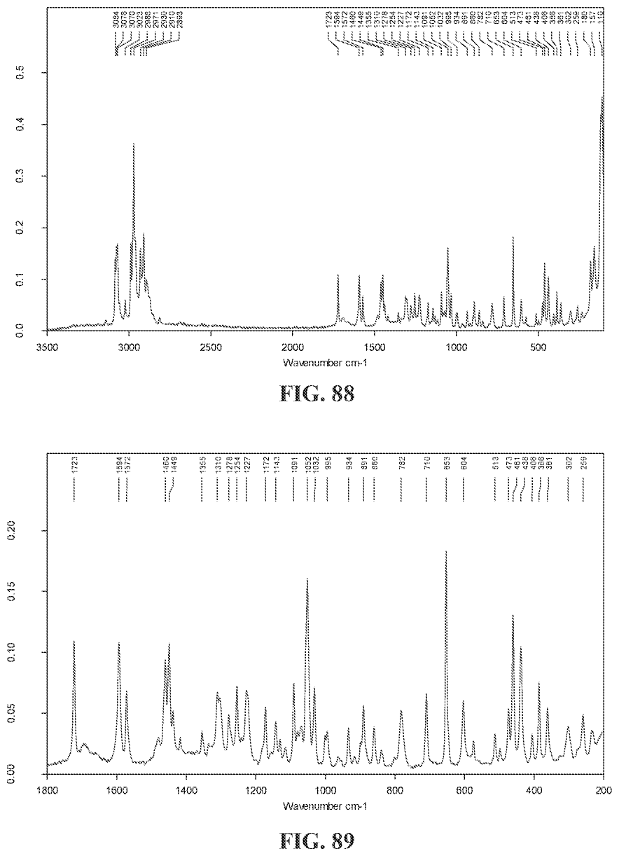

In some embodiments, the R-ketamine D-tartrate salt Form D is a crystalline polymorphic form characterized by a PXRD spectrum substantially similar to that shown in FIG. 87. In some embodiments, the R-ketamine D-tartrate salt Form D is a crystalline polymorphic form characterized by an FT-Raman spectrum substantially similar to that shown in FIG. 88. In some embodiments, the R-ketamine D-tartrate salt Form D is an ethyl acetate solvate. In some embodiments, the R-ketamine D-tartrate salt Form D is a methanol solvate. In some embodiments, the R-ketamine D-tartrate salt Form D is a THF solvate. In some embodiments, the R-ketamine D-tartrate salt Form D is a propanol solvate. In some embodiments, the R-ketamine D-tartrate salt Form D is an acetonitrile solvate. In some embodiments, the R-ketamine D-tartrate salt Form D is an acetone solvate. In some embodiments, the R-ketamine D-tartrate salt Form D is a hydrate. In some embodiments, the R-ketamine D-tartrate salt Form D is a methanol:ethyl acetate solvate. In some embodiments, the R-ketamine D-tartrate salt Form D is anhydrous. In some embodiments, the R-ketamine D-tartrate salt Form D is a crystalline polymorphic form characterized by a ¹H-NMR according to FIG. 95.

In some embodiments, the R-ketamine D-tartrate salt Form D is a 1:1 R-ketamine:D-tartaric acid salt. In some embodiments, the R-ketamine D-tartrate salt Form D is a 1:1.25 R-ketamine:D-tartaric acid salt. In some embodiments, the R-ketamine D-tartrate salt Form D is a 1:1.5 R-ketamine:D-tartaric acid salt. In some embodiments, the R-ketamine D-tartrate salt Form D is a 1:0.85 R-ketamine:D-tartaric acid salt. In some embodiments, the R-ketamine D-tartrate salt Form D is a 2:1 R-ketamine:D-tartaric acid salt. In some embodiments, the R-ketamine D-tartrate salt Form D is a 1:2 R-ketamine:D-tartaric acid salt.

In some embodiments, the R-ketamine D-tartrate salt is a crystalline polymorphic Form D characterized by a PXRD peak at 14.3 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by a PXRD peak at 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at 14.3 °2θ and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 12.7 °2θ, 14.3 °2θ, and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at 12.7 °2θ, 14.3 °2θ, and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 12.7 °2θ, 14.3 °2θ, 14.8 °2θ, and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 12.7 °2θ, 14.3 °2θ, 15.1 °2θ, and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at 12.7 °2θ, 14.3 °2θ, 14.8 °2θ, and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at 12.7 °2θ, 14.3 °2θ, 15.1 °2θ, and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 12.7 °2θ, 14.3 °2θ, 14.8 °2θ, 15.1 °2θ, and 19.8 °2θ (±0.2 °2θ; ±0.1 °2θ; or 0.0 °2θ; Cu Kα1 radiation).

In some embodiments, the R-ketamine D-tartrate salt is a crystalline polymorphic Form D characterized by PXRD peaks at 12.7 °2θ, 14.3 °2θ, 14.8 °2θ, 15.1 °2θ, and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 12.7 °2θ, 14.3 °2θ, 14.8 °2θ, 15.1 °2θ, 17.0 °2θ, and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at 12.7 °2θ, 14.3 °2θ, 14.8 °2θ, 15.1 °2θ, 17.0 °2θ, and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 7.6 °2θ, 12.7 °2θ, 14.3 °2θ, 14.8 °2θ, 15.1 °2θ, 17.0 °2θ, and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at 7.6 °2θ, 12.7 °2θ, 14.3 °2θ, 14.8 °2θ, 15.1 °2θ, 17.0 °2θ, and 19.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 7.6 °2θ, 12.7 °2θ, 14.3 °2θ, 14.8 °2θ, 15.1 °2θ, 17.0 °2θ, 19.8 °2θ, and 21.5 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 7.6 °2θ, 12.7 °2θ, 14.3 °2θ, 14.8 °2θ, 15.1 °2θ, 17.0 °2θ, 19.8 °2θ, and 22.7 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 7.6 °2θ, 12.7 °2θ, 14.3 °2θ, 14.8 °2θ, 15.1 °2θ, 17.0 °2θ, 19.8 °2θ, and 23.9 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 7.6 °2θ, 12.7 °2θ, 14.3 °2θ, 14.8 °2θ, 15.1 °2θ, 17.0 °2θ, 19.8 °2θ, and 24.8 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 7.6 °2θ, 12.7 °2θ, 14.3 °2θ, 14.8 °2θ, 15.1 °2θ, 17.0 °2θ, 19.8 °2θ, and 33.1 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks at two or more, or three or more peaks selected from the group consisting of 7.6 °2θ, 12.7 °2θ, 14.3 °2θ, 14.8 °2θ, 15.1 °2θ, 17.0 °2θ, 19.8 °2θ, and 38.0 °2θ (±0.2 °2θ; ±0.1 °2θ; or ±0.0 °2θ; Cu Kα1 radiation).

In some embodiments, the R-ketamine D-tartrate salt is a crystalline polymorphic Form D characterized by PXRD peaks 7.6 °2θ, 12.7 °2θ, 14.3 °2θ, 14.8 °2θ, 15.1 °2θ, 17.0 °2θ, and 19.8 °2θ, in addition to one or more, two or more, or three or more peaks selected from the group consisting of 21.5 °2θ, 22.7 °2θ, 23.9 °2θ, 24.8 °2θ, 27.4 °2θ, 27.5 °2θ, 33.1 °2θ, or 38.0 °2θ, (±0.2 °2θ; ±0.1 °2θ; or ±0.0 °2θ; Cu Kα1 radiation).

In some embodiments, the R-ketamine D-tartrate salt Form D is characterized by one, two, three, four, five, six, seven, eight, nine, ten, eleven, twelve, thirteen, fourteen, fifteen, or sixteen PXRD peaks selected from those set forth in Table 11.

In some embodiments, the R-Ketamine D-tartrate salt Form D is characterized by one or more peaks selected from those set forth in Table 11 that have a relative intensity of over 20%.

In some embodiments, the R-Ketamine D-tartrate salt Form D is characterized by two or more peaks selected from those set forth in Table 11 that have a relative intensity of over 20%.

In some embodiments, the R-Ketamine D-tartrate salt Form D is characterized by three or more peaks selected from those set forth in Table 11 that have a relative intensity of over 20%.

In some embodiments, the R-Ketamine D-tartrate salt Form D is characterized by four or more peaks selected from those set forth in Table 11 that have a relative intensity of over 20%.

In some embodiments, the R-Ketamine D-tartrate salt Form D is characterized by five or more peaks selected from those set forth in Table 11 that have a relative intensity of over 20%.

In some embodiments, the R-Ketamine D-tartrate salt Form D is characterized by six or more peaks selected from those set forth in Table 11 that have a relative intensity of over 20%.

TABLE 11
R-ketamine D-tartrate Salt Form D PXRD (±0.2 °2θ; ±0.1 °2θ;
or ±0.0 °2θ; Cu Kα1 radiation).

| Angle in | d-spacing | | |
|---|---|---|---|
| °2-theta | in Å | Intensity | Intensity % |
| 7.57 | 11.66 | s | 43 |
| 8.42 | 10.49 | m | 27 |
| 12.29 | 7.20 | m | 22 |
| 12.72 | 6.95 | vs | 87 |
| 13.43 | 6.59 | m | 25 |
| 14.32 | 6.18 | vs | 100 |
| 14.82 | 5.97 | s | 62 |
| 15.08 | 5.87 | s | 62 |
| 15.56 | 5.69 | m | 17 |
| 15.78 | 5.61 | m | 18 |
| 16.21 | 5.46 | m | 23 |
| 16.61 | 5.33 | m | 23 |
| 16.98 | 5.22 | s | 54 |
| 17.95 | 4.94 | m | 16 |
| 18.33 | 4.84 | m | 24 |
| 18.92 | 4.69 | m | 20 |
| 19.52 | 4.54 | m | 26 |
| 19.78 | 4.49 | vs | 100 |
| 20.71 | 4.28 | m | 26 |
| 20.93 | 4.24 | m | 22 |
| 21.48 | 4.13 | s | 33 |
| 22.65 | 3.92 | s | 33 |
| 22.85 | 3.89 | m | 25 |
| 23.94 | 3.71 | s | 33 |
| 24.40 | 3.64 | w | 14 |
| 24.76 | 3.59 | s | 33 |
| 25.58 | 3.48 | w | 11 |
| 25.83 | 3.45 | m | 17 |
| 26.10 | 3.41 | m | 16 |
| 26.73 | 3.33 | w | 15 |
| 26.97 | 3.30 | m | 17 |
| 27.36 | 3.26 | s | 31 |
| 27.50 | 3.24 | s | 30 |
| 28.31 | 3.15 | m | 20 |
| 28.72 | 3.11 | w | 10 |
| 29.10 | 3.07 | w | 9 |
| 29.87 | 2.99 | m | 17 |
| 30.21 | 2.96 | w | 14 |
| 30.42 | 2.94 | w | 12 |
| 30.71 | 2.91 | m | 17 |
| 31.11 | 2.87 | m | 18 |
| 31.40 | 2.85 | m | 26 |
| 32.30 | 2.77 | w | 15 |
| 32.47 | 2.75 | m | 24 |
| 32.77 | 2.73 | m | 24 |
| 33.12 | 2.70 | s | 32 |
| 33.32 | 2.69 | m | 22 |
| 33.63 | 2.66 | w | 14 |
| 33.78 | 2.65 | w | 12 |
| 34.05 | 2.63 | w | 9 |
| 34.57 | 2.59 | w | 11 |
| 34.82 | 2.57 | w | 14 |
| 35.60 | 2.52 | w | 13 |
| 36.65 | 2.45 | w | 9 |
| 36.96 | 2.43 | w | 13 |
| 37.18 | 2.42 | m | 16 |
| 37.57 | 2.39 | w | 8 |
| 38.04 | 2.36 | s | 32 |
| 38.28 | 2.35 | w | 11 |
| 38.65 | 2.33 | w | 9 |
| 38.87 | 2.31 | w | 12 |
| 39.62 | 2.27 | w | 8 |

### R-Ketamine Oxalate Salt

In one aspect, the present disclosure provides an R-ketamine oxalate salt. In some embodiments, the R-ketamine oxalate salt is amorphous. In some embodiments, the R-ketamine oxalate salt is crystalline. In some embodiments, the R-ketamine oxalate salt is a crystalline polymorphic form. In some embodiments, the R-ketamine oxalate salt is a crystalline polymorphic form characterized by the PXRD peaks set forth below in Table 12.

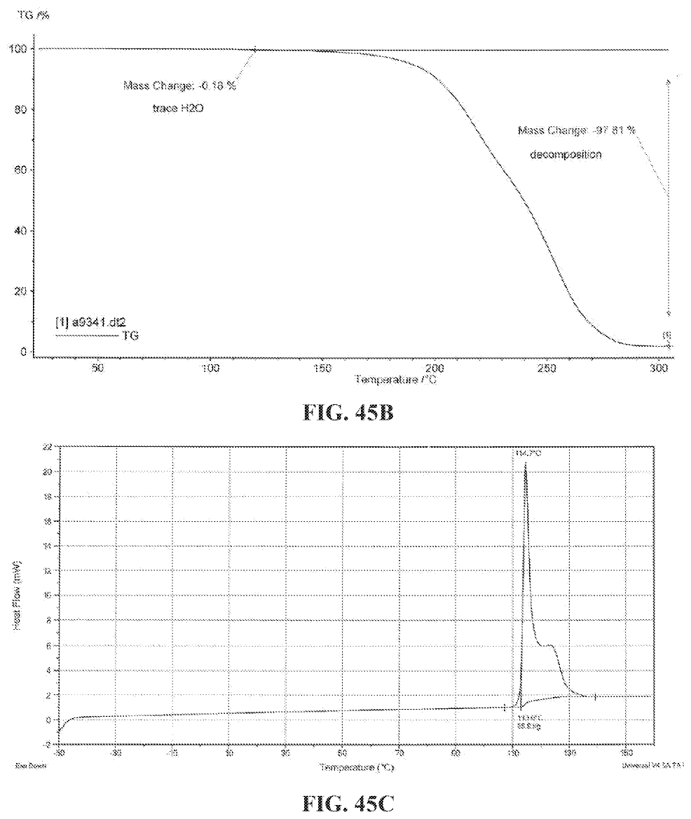

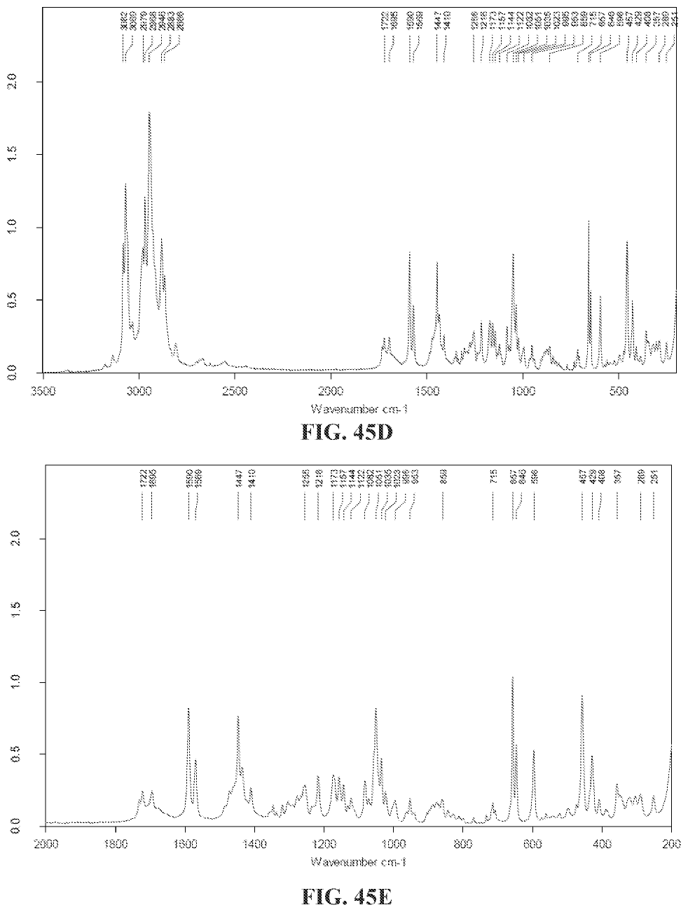

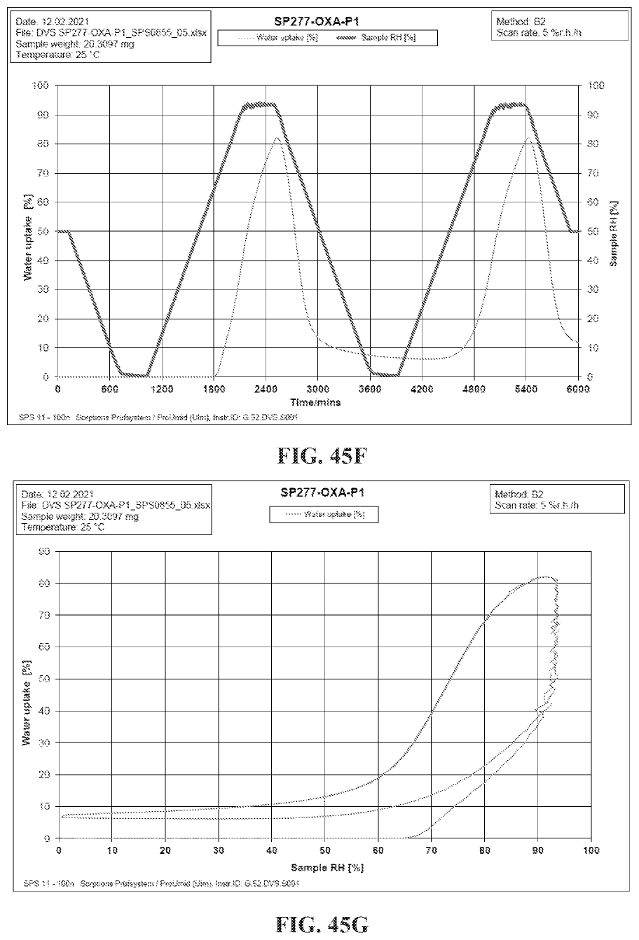

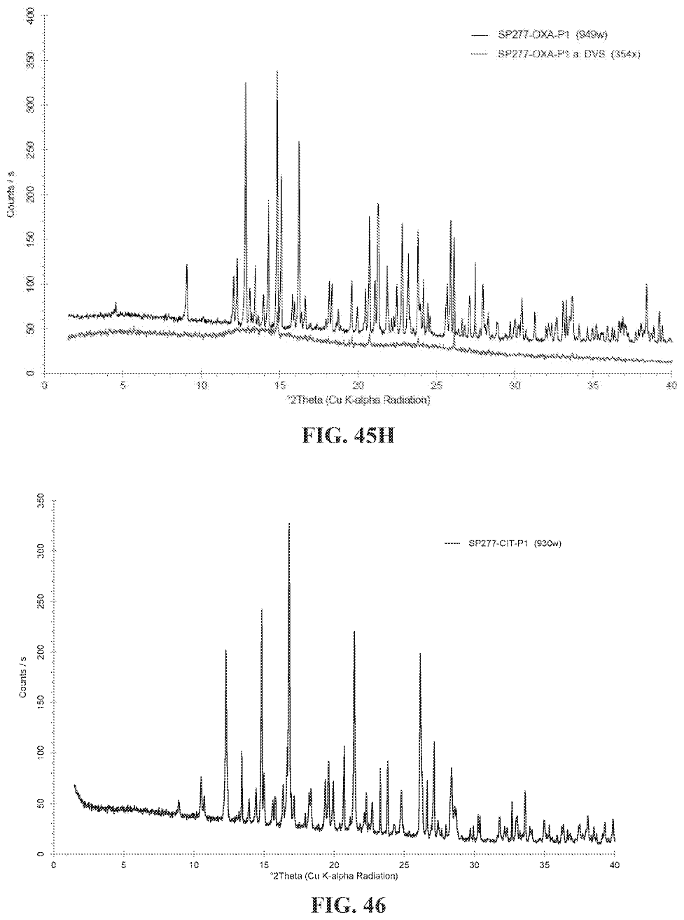

In some embodiments, the R-ketamine oxalate salt is a crystalline polymorphic form characterized by PXRD spectrum substantially similar to that shown in FIG. 43. In some embodiments, the R-ketamine oxalate salt is a crystalline polymorphic form characterized by an FT-Raman spectrum substantially similar to that shown in FIG. 45C. In some embodiments, the R-ketamine oxalate salt is a crystalline polymorphic form characterized by a DSC thermogram substantially similar to that of FIG. 45C. In some embodiments, the R-ketamine oxalate salt is a crystalline polymorphic form characterized by a DSC thermogram with a broad endothermic signal with a maximum at 115° C.

In some embodiments, the R-ketamine oxalate salt is a solvate. In some embodiments, the R-ketamine oxalate salt is a hydrate. In some embodiments, the R-ketamine oxalate salt is anhydrous.

In some embodiments, the R-ketamine oxalate salt is a 1:1 R-ketamine:oxalate salt. In some embodiments, the R-ketamine oxalate salt is a 1:2 R-ketamine:oxalate salt.

In some embodiments, the R-ketamine oxalate salt is a crystalline polymorphic form characterized by a PXRD peak at 14.8 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 12.8 °2θ and 14.8 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 12.8 °2θ, 14.8 °2θ and 16.2 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 12.8 °2θ, 14.8 °2θ and 16.2 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 12.8 °2θ, 14.8 °2θ, 15.1 °2θ, and 16.2 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 12.8 °2θ, 14.8 °2θ, 15.1 °2θ, and 16.2 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 12.8 °2θ, 14.3 °2θ, 14.8 °2θ, 15.1 °2θ, and 16.2 °2θ (±0.2 °2θ; ±0.1 °2θ; or 0.0 °2θ; Cu Kα1 radiation).

In some embodiments, the R-ketamine oxalate salt is a crystalline polymorphic form characterized by PXRD peaks at 12.8 °2θ, 14.3 °2θ, 14.8 °2θ, 15.1 °2θ, and 16.2 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 12.8 °2θ, 14.3 °2θ, 14.8 °2θ, 15.1 °2θ, 16.2 °2θ, and 21.3 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 12.8 °2θ, 14.3 °2θ, 14.8 °2θ, 15.1 °2θ, 16.2 °2θ, and 21.3 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 12.8 °2θ, 14.3 °2θ, 14.8 °2θ, 15.1 °2θ, 16.2 °2θ, 20.7 °2θ, and 21.3 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 12.8 °2θ, 14.3 °2θ, 14.8 °2θ, 15.1 °2θ, 16.2 °2θ, 20.7 °2θ, and 21.3 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 12.8 °2θ, 14.3 °2θ, 14.8 °2θ, 15.1 °2θ, 16.2 °2θ, 20.7 °2θ, 21.3 °2θ, and 25.9 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 12.8 °2θ, 14.3 °2θ, 14.8 °2θ, 15.1 °2θ, 16.2 °2θ, 20.7 °2θ, 21.3 °2θ, and 25.9 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 12.8 °2θ, 14.3 °2θ, 14.8 °2θ, 15.1 °2θ, 16.2 °2θ, 20.7 °2θ, 21.3 °2θ, 22.8 °2θ, and 25.9 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 12.8 °2θ, 14.3 °2θ, 14.8 °2θ, 15.1 °2θ, 16.2 °2θ, 20.7 °2θ, 21.3 °2θ, 22.8 °2θ, and 25.9 °2θ (10.2 °2θ; 10.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 12.8 °2θ, 14.3 °2θ, 14.8 °2θ, 15.1 °2θ, 16.2 °2θ, 20.7 °2θ, 21.3 °2θ, 22.8 °2θ, 23.8 °2θ, and 25.9 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 12.8 °2θ, 14.3 °2θ, 14.8 °2θ, 15.1 °2θ, 16.2 °2θ, 20.7 °2θ, 21.3 °2θ, 22.8 °2θ, 23.8 °2θ, and 25.9 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 12.8 °2θ, 14.3 °2θ, 14.8 °2θ, 15.1 °2θ, 16.2 °2θ, 20.7 °2θ, 21.3 °2θ, 22.8 °2θ, 23.8 °2θ, 25.9 °2θ, and 26.1 °2θ (±0.2 °2θ; ±0.1 °2θ; or 0.0 °2θ; Cu Kα1 radiation).

In some embodiments, the R-ketamine oxalate salt is a crystalline polymorphic form characterized by PXRD peaks at 12.8 °2θ, 14.3 °2θ, 14.8 °2θ, 15.1 °2θ, 16.2 °2θ, 20.7 °2θ, 21.3 °2θ, 22.8 °2θ, 23.8 °2θ, 25.9 °2θ, and 26.1 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 12.8 °2θ, 14.3 °2θ, 14.8 °2θ, 15.1 °2θ, 16.2 °2θ, 20.7 °2θ, 21.3 °2θ, 22.8 °2θ, 23.2 °2θ, 23.8 °2θ, 25.9 °2θ, and 26.1 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 12.8 °2θ, 14.3 °2θ, 14.8 °2θ, 15.1 °2θ, 16.2 °2θ, 20.7 °2θ, 21.3 °2θ, 22.8 °2θ, 23.2 °2θ, 23.8 °2θ, 25.9 °2θ, and 26.1 °2θ (±0.2 °2θ; 0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 12.3 °2θ, 12.8 °2θ, 14.3 °2θ, 14.8 °2θ, 15.1 °2θ, 16.2 °2θ, 20.7 °2θ, 21.3 °2θ, 22.8 °2θ, 23.2 °2θ, 23.8 °2θ, 25.9 °2θ, and 26.1 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 12.3 °2θ, 12.8 °2θ, 14.3 °2θ, 14.8 °2θ, 15.1 °2θ, 16.2 °2θ, 20.7 °2θ, 21.3 °2θ, 22.8 °2θ, 23.2 °2θ, 23.8 °2θ, 25.9 °2θ, and 26.1 °2θ (±0.2 °2θ; ±0.1 °2θ; or 0.0 °2θ; Cu Kα1 radiation).

In some embodiments, the R-ketamine oxalate salt is a crystalline polymorphic form characterized by PXRD peaks at two or more, or three or more peaks selected from the group consisting of 12.3 °2θ, 12.8 °2θ, 14.3 °2θ, 14.8 °2θ, 15.1 °2θ, 16.2 °2θ, 20.7 °2θ, 21.3 °2θ, 22.8 °2θ, 23.2 °2θ, 23.8 °2θ, 25.9 °2θ, 26.1 °2θ, and 27.5 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 12.3 °2θ, 12.8 °2θ, 14.3 °2θ, 14.8 °2θ, 15.1 °2θ, 16.2 °2θ, 20.7 °2θ, 21.3 °2θ, 22.8 °2θ, 23.2 °2θ, 23.8 °2θ, 25.9 °2θ, 26.1 °2θ, and 27.5 °2θ (±0.2 °2θ; ±0.1 °2θ; or +0.0 °2θ; Cu Kα1 radiation).

In some embodiments, the R-ketamine oxalate salt is a crystalline polymorphic form characterized by PXRD peaks at two or more, or three or more peaks selected from the group consisting of 9.1 °2θ, 12.3 °2θ, 12.8 °2θ, 14.3 °2θ, 14.8 °2θ, 15.1 °2θ, 16.2 °2θ, 20.7 °2θ, 21.3 °2θ, 22.8 °2θ, 23.2 °2θ, 23.8 °2θ, 25.9 °2θ, 26.1 °2θ, and 27.5 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 9.1 °2θ, 12.3 °2θ, 12.8 °2θ, 14.3 °2θ, 14.8 °2θ, 15.1 °2θ, 16.2 °2θ, 20.7 °2θ, 21.3 °2θ, 22.8 °2θ, 23.2 °2θ, 23.8 °2θ, 25.9 °2θ, 26.1 °2θ, and 27.5 °2θ (±0.2 °2θ; 15±0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 9.1 °2θ, 12.3 °2θ, 12.8 °2θ, 13.4 °2θ, 14.3 °2θ, 14.8 °2θ, 15.1 °2θ, 16.2 °2θ, 20.7 °2θ, 21.3 °2θ, 22.8 °2θ, 23.2 °2θ, 23.8 °2θ, 25.9 °2θ, 26.1 °2θ, and 27.5 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 9.1 °2θ, 12.3 °2θ, 12.8 °2θ, 13.4 °2θ, 14.3 °2θ, 14.8 °2θ, 15.1 °2θ, 16.2 °2θ, 20.7 °2θ, 21.3 °2θ, 22.8 °2θ, 23.2 °2θ, 23.8 °2θ, 25.9 °2θ, 26.1 °2θ, and 27.5 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at two or more, or three or more peaks selected from the group consisting of 9.1 °2θ, 12.3 °2θ, 12.8 °2θ, 13.4 °2θ, 14.3 °2θ, 14.8 °2θ, 15.1 °2θ, 16.2 °2θ, 20.7 °2θ, 21.3 °2θ, 21.9 °2θ, 22.8 °2θ, 23.2 °2θ, 23.8 °2θ, 25.9 °2θ, 26.1 °2θ, and 27.5 °2θ (±0.2 °2θ; ±0.1 °2θ; or ±0.0 °2θ; Cu Kα1 radiation).

In some embodiments, the R-ketamine oxalate salt is a crystalline polymorphic form characterized by PXRD peaks at 9.1 °2θ, 12.3 °2θ, 12.8 °2θ, 13.4 °2θ, 14.3 °2θ, 14.8 °2θ, 15.1 °2θ, 16.2 °2θ, 20.7 °2θ, 21.3 °2θ, 21.9 °2θ, 22.8 °2θ, 23.2 °2θ, 23.8 °2θ, 25.9 °2θ, 26.1 °2θ, and 27.5 °2θ (±0.2 °2θ; ±0.1 °2θ; or +0.0 °2θ; Cu Kα1 radiation).

In some embodiments, the R-ketamine oxalate salt is a crystalline polymorphic form characterized by one, two, three, four, five, six, seven, eight, nine, ten, eleven, twelve, thirteen, fourteen, fifteen, or sixteen PXRD peaks selected from those set forth in Table 12.

TABLE 12
R-ketamine Oxalate Salt PXRD (±0.2 °2θ; ±0.1 °2θ;
or ±0.0° 2θ; Cu Kα1 radiation).

| Angle in °2-theta | d-spacing in Å | Intensity | Intensity % |
|---|---|---|---|
| 4.5 | 19.63 | m | 16 |
| 9.1 | 9.76 | s | 31 |
| 12.0 | 7.35 | m | 27 |
| 12.3 | 7.21 | s | 33 |
| 12.8 | 6.90 | vs | 96 |
| 13.1 | 6.76 | m | 22 |
| 13.4 | 6.59 | s | 31 |
| 14.0 | 6.34 | m | 20 |
| 14.3 | 6.20 | s | 54 |
| 14.8 | 5.97 | vs | 100 |
| 15.1 | 5.87 | s | 63 |
| 15.9 | 5.59 | m | 19 |
| 16.2 | 5.46 | vs | 75 |
| 16.6 | 5.33 | m | 20 |
| 16.9 | 5.23 | w | 10 |
| 18.2 | 4.88 | m | 25 |
| 18.7 | 4.75 | w | 13 |
| 19.6 | 4.53 | m | 25 |
| 19.9 | 4.45 | m | 16 |
| 20.4 | 4.34 | m | 22 |
| 20.7 | 4.28 | s | 48 |
| 21.0 | 4.23 | w | 11 |
| 21.3 | 4.17 | s | 53 |
| 21.9 | 4.06 | s | 30 |
| 22.2 | 4.00 | w | 11 |
| 22.5 | 3.96 | m | 24 |
| 22.8 | 3.90 | s | 46 |
| 23.2 | 3.83 | s | 35 |
| 23.8 | 3.73 | s | 43 |
| 24.2 | 3.68 | m | 26 |
| 24.5 | 3.63 | m | 17 |
| 25.7 | 3.47 | m | 24 |
| 25.9 | 3.44 | s | 47 |
| 26.1 | 3.41 | s | 41 |
| 26.4 | 3.38 | w | 8 |
| 26.6 | 3.35 | w | 12 |
| 27.1 | 3.29 | m | 20 |
| 27.5 | 3.24 | s | 32 |
| 28.0 | 3.19 | m | 24 |
| 28.3 | 3.15 | w | 14 |
| 28.9 | 3.09 | w | 11 |
| 29.4 | 3.03 | w | 6 |
| 29.7 | 3.01 | w | 11 |
| 30.0 | 2.98 | w | 12 |
| 30.5 | 2.93 | m | 19 |
| 30.7 | 2.91 | w | 8 |
| 31.3 | 2.86 | w | 14 |
| 32.1 | 2.79 | w | 7 |
| 32.3 | 2.77 | w | 7 |
| 32.6 | 2.74 | w | 10 |
| 33.1 | 2.70 | m | 16 |
| 33.6 | 2.66 | m | 19 |
| 34.1 | 2.63 | w | 10 |
| 34.6 | 2.59 | w | 9 |
| 34.9 | 2.57 | w | 8 |
| 35.2 | 2.55 | w | 10 |
| 35.6 | 2.52 | w | 7 |
| 35.9 | 2.50 | w | 9 |
| 36.3 | 2.48 | w | 7 |
| 36.7 | 2.45 | w | 9 |
| 37.0 | 2.43 | w | 10 |
| 37.7 | 2.38 | w | 7 |
| 38.0 | 2.36 | w | 10 |
| 38.4 | 2.34 | m | 24 |
| 38.8 | 2.32 | w | 9 |
| 39.2 | 2.30 | w | 14 |
| 39.4 | 2.28 | w | 9 |
| 39.9 | 2.26 | vw | 5 |
| 40.5 | 2.23 | vw | 4 |

### R-Ketamine Citrate Salt

In one aspect, the present disclosure provides an R-ketamine citrate salt. In some embodiments, the R-ketamine citrate salt is amorphous. In some embodiments, the R-ketamine citrate salt is crystalline. In some embodiments, the R-ketamine citrate salt is a crystalline polymorphic form. In some embodiments, the R-ketamine citrate salt is a crystalline polymorphic form characterized by the PXRD peaks set forth below in Table 13.

In some embodiments, the R-ketamine citrate salt is a crystalline polymorphic form characterized by a PXRD spectrum substantially similar to that shown in FIG. 46. In some embodiments, the R-ketamine citrate salt is a crystalline polymorphic form characterized by a ¹H-NMR spectrum substantially similar to that shown in FIG. 48.

In some embodiments, the R-ketamine citrate salt is a solvate. In some embodiments, the R-ketamine citrate salt is a hydrate. In some embodiments, the R-ketamine citrate salt is anhydrous.

In some embodiments, the R-ketamine citrate salt is monocitrate salt. In some embodiments, the R-ketamine citrate salt is a 1:2 R-ketamine:citrate salt. In some embodiments, the R-ketamine citrate salt is a 2:1 R-ketamine:citrate salt. In some embodiments, the R-ketamine citrate salt is a 1:1 R-ketamine:citrate salt.

In some embodiments, the R-ketamine citrate salt is a crystalline polymorphic form characterized by a PXRD peak at 16.8 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 14.8 °2θ and 16.8 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three peaks selected from the group consisting of 14.8 °2θ, 16.8 °2θ, and 21.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 14.8 °2θ, 16.8 °2θ, and 21.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 12.3 °2θ, 14.8 °2θ, 16.8 °2θ, and 21.4 °2θ (10.2 °2θ; 10.1 °2θ; or 0.0 °2θ; Cu Kα1 radiation).

In some embodiments, the R-ketamine citrate salt is a crystalline polymorphic form characterized by PXRD peaks at 12.3 °2θ, 14.8 °2θ, 16.8 °2θ, and 21.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 12.3 °2θ, 14.8 °2θ, 16.8 °2θ, 21.4 °2θ, and 26.2 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 12.3 °2θ, 14.8 °2θ, 16.8 °2θ, 21.4 °2θ, and 26.2 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 12.3 °2θ, 14.8 °2θ, 16.8 °2θ, 20.7 °2θ, 21.4 °2θ, and 26.2 °2θ (±0.2 °2θ; ±0.1 °2θ; or +0.0 °2θ; Cu Kα1 radiation).

In some embodiments, the R-ketamine citrate salt is a crystalline polymorphic form characterized by PXRD peaks at 12.3 °2θ, 14.8 °2θ, 16.8 °2θ, 20.7 °2θ, 21.4 °2θ, and 26.2 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 12.3 °2θ, 13.4 °2θ, 14.8 °2θ, 16.8 °2θ, 20.7 °2θ, 21.4 °2θ, and 26.2 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 12.3 °2θ, 13.4 °2θ, 14.8 °2θ, 16.8 °2θ, 20.7 °2θ, 21.4 °2θ, and 26.2 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 12.3 °2θ, 13.4 °2θ, 14.8 °2θ, 16.8 °2θ, 20.7 °2θ, 21.4 °2θ, 26.2 °2θ, and 27.1 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 12.3 °2θ, 13.4 °2θ, 14.8 °2θ, 16.8 °2θ, 20.7 °2θ, 21.4 °2θ, 26.2 °2θ, and 27.1 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 12.3 °2θ, 13.4 °2θ, 14.8 °2θ, 16.8 °2θ, 19.6 °2θ, 20.7 °2θ, 21.4 °2θ, 26.2 °2θ, and 27.1 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 12.3 °2θ, 13.4 °2θ, 14.8 °2θ, 16.8 °2θ, 19.6 °2θ, 20.7 °2θ, 21.4 °2θ, 26.2 °2θ, and 27.1 °2θ (±0.2 °2θ; ±0.1 °2θ; or 0.0 °2θ; Cu Kα1 radiation).

In some embodiments, the R-ketamine citrate salt is a crystalline polymorphic form characterized by PXRD peaks at two or more, or three or more peaks selected from the group consisting of 12.3 °2θ, 13.4 °2θ, 14.8 °2θ, 16.8 °2θ, 19.6 °2θ, 20.7 °2θ, 21.4 °2θ, 23.8 °2θ, 26.2 °2θ, and 27.1 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 12.3 °2θ, 13.4 °2θ, 14.8 °2θ, 16.8 °2θ, 19.6 °2θ, 20.7 °2θ, 21.4 °2θ, 23.8 °2θ, 26.2 °2θ, and 27.1 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 12.3 °2θ, 13.4 °2θ, 14.8 °2θ, 16.8 °2θ, 19.6 °2θ, 20.7 °2θ, 21.4 °2θ, 23.8 °2θ, 26.2 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 12.3 °2θ, 13.4 °2θ, 14.8 °2θ, 16.8 °2θ, 19.6 °2θ, 20.7 °2θ, 21.4 °2θ, 23.8 °2θ, 26.2 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 12.3 °2θ, 13.4 °2θ, 14.8 °2θ, 16.8 °2θ, 19.6 °2θ, 20.7 °2θ, 21.4 °2θ, 23.3 °2θ, 23.8 °2θ, 26.2 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 12.3 °2θ, 13.4 °2θ, 14.8 °2θ, 16.8 °2θ, 19.6 °2θ, 20.7 °2θ, 21.4 °2θ, 23.3 °2θ, 23.8 °2θ, 26.2 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 10.5 °2θ, 12.3 °2θ, 13.4 °2θ, 14.8 °2θ, 16.8 °2θ, 19.6 °2θ, 20.7 °2θ, 21.4 °2θ, 23.3 °2θ, 23.8 °2θ, 26.2 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 10.5 °2θ, 12.3 °2θ, 13.4 °2θ, 14.8 °2θ, 16.8 °2θ, 19.6 °2θ, 20.7 °2θ, 21.4 °2θ, 23.3 °2θ, 23.8 °2θ, 26.2 °2θ, 27.1 °2θ, and 28.4 °2θ (±0.2 °2θ; ±0.1 °2θ; or 0.0 °2θ; Cu Kα1 radiation).

In some embodiments, the R-ketamine citrate salt is a crystalline polymorphic form characterized by PXRD peaks at two or more, or three or more peaks selected from the group consisting of 10.5 °2θ, 12.3 °2θ, 13.4 °2θ, 14.8 °2θ, 16.8 °2θ, 19.4 °2θ, 19.6 °2θ, 20.7 °2θ, 21.4 °2θ, 23.3 °2θ, 23.8 °2θ, 26.2 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 10.5 °2θ, 12.3 °2θ, 13.4 °2θ, 14.8 °2θ, 16.8 °2θ, 19.4 °2θ, 19.6 °2θ, 20.7 °2θ, 21.4 °2θ, 23.3 °2θ, 23.8 °2θ, 26.2 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 10.5 °2θ, 12.3 °2θ, 13.4 °2θ, 14.8 °2θ, 16.8 °2θ, 19.4 °2θ, 19.6 °2θ, 20.7 °2θ, 21.4 °2θ, 23.3 °2θ, 23.8 °2θ, 26.2 °2θ, 26.6 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 10.5 °2θ, 12.3 °2θ, 13.4 °2θ, 14.8 °2θ, 16.8 °2θ, 19.4 °2θ, 19.6 °2θ, 20.7 °2θ, 21.4 °2θ, 23.3 °2θ, 23.8 °2θ, 26.2 °2θ, 26.6 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 10.5 °2θ, 12.3 °2θ, 13.4 °2θ, 14.8 °2θ, 16.8 °2θ, 19.4 °2θ, 19.6 °2θ, 19.9 °2θ, 20.7 °2θ, 21.4 °2θ, 23.3 °2θ, 23.8 °2θ, 26.2 °2θ, 26.6 °2θ, 27.1 °2θ, and 28.4 °2θ (±0.2 °2θ; ±0.1 °2θ; or +0.0 °2θ; Cu Kα1 radiation).

In some embodiments, the R-ketamine citrate salt is a crystalline polymorphic form characterized by PXRD peaks at 10.5 °2θ, 12.3 °2θ, 13.4 °2θ, 14.8 °2θ, 16.8 °2θ, 19.4 °2θ, 19.6 °2θ, 19.9 °2θ, 20.7 °2θ, 21.4 °2θ, 23.3 °2θ, 23.8 °2θ, 26.2 °2θ, 26.6 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 10.5 °2θ, 12.3 °2θ, 13.4 °2θ, 14.8 °2θ, 16.5 °2θ, 16.8 °2θ, 19.4 °2θ, 19.6 °2θ, 19.9 °2θ, 20.7 °2θ, 21.4 °2θ, 23.3 °2θ, 23.8 °2θ, 26.2 °2θ, 26.6 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 10.5 °2θ, 12.3 °2θ, 13.4 °2θ, 14.8 °2θ, 16.5 °2θ, 16.8 °2θ, 19.4 °2θ, 19.6 °2θ, 19.9 °2θ, 20.7 °2θ, 21.4 °2θ, 23.3 °2θ, 23.8 °2θ, 26.2 °2θ, 26.6 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at two or more, or three or more peaks selected from the group consisting of 10.5 °2θ, 12.3 °2θ, 13.4 °2θ, 14.4 °2θ, 14.8 °2θ, 16.5 °2θ, 16.8 °2θ, 19.4 °2θ, 19.6 °2θ, 19.9 °2θ, 20.7 °2θ, 21.4 °2θ, 23.3 °2θ, 23.8 °2θ, 26.2 °2θ, 26.6 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by PXRD peaks at 10.5 °2θ, 12.3 °2θ, 13.4 °2θ, 14.4 °2θ, 14.8 °2θ, 16.5 °2θ, 16.8 °2θ, 19.4 °2θ, 19.6 °2θ, 19.9 °2θ, 20.7 °2θ, 21.4 °2θ, 23.3 °2θ, 23.8 °2θ, 26.2 °2θ, 26.6 °2θ, 27.1 °2θ, and 28.4 °2θ (±0.2 °2θ; ±0.1 °2θ; or ±0.0 °2θ; Cu Kα1 radiation).

In some embodiments, the R-ketamine citrate salt is a crystalline polymorphic form characterized by one, two, three, four, five, six, seven, eight, nine, ten, eleven, twelve, thirteen, fourteen, fifteen, or sixteen PXRD peaks selected from those set forth in Table 13.

TABLE 13
R-ketamine Citrate Salt PXRD (±0.2 °2θ; ±0.1 °2θ;
or ±0.0° 2θ; Cu Kα1 radiation).

| Angle in °2-theta | d-spacing in Å | Intensity | Intensity % |
|---|---|---|---|
| 8.9 | 9.90 | m | 16.2 |
| 10.5 | 8.40 | m | 23.3 |
| 10.7 | 8.24 | m | 17.6 |
| 12.3 | 7.19 | s | 61.7 |
| 13.4 | 6.59 | s | 31 |
| 13.9 | 6.35 | m | 16.7 |
| 14.4 | 6.14 | m | 20 |
| 14.8 | 5.96 | vs | 74 |
| 15.1 | 5.88 | m | 17.5 |
| 15.6 | 5.68 | m | 15 |
| 15.8 | 5.60 | m | 17.4 |
| 16.5 | 5.41 | m | 21 |
| 16.8 | 5.27 | vs | 100 |
| 17.1 | 5.17 | m | 17.6 |
| 17.9 | 4.94 | w | 12.3 |
| 18.3 | 4.84 | m | 17.5 |
| 18.9 | 4.70 | w | 8.9 |
| 19.4 | 4.58 | m | 22.4 |
| 19.6 | 4.53 | m | 28.1 |
| 19.9 | 4.45 | m | 22 |
| 20.7 | 4.28 | s | 32.6 |
| 21.1 | 4.20 | w | 11.2 |
| 21.4 | 4.14 | s | 67.4 |
| 22.3 | 3.99 | m | 18.7 |
| 22.7 | 3.91 | m | 15.6 |
| 23.3 | 3.82 | m | 25.8 |
| 23.8 | 3.73 | m | 28.1 |
| 24.3 | 3.66 | w | 8.9 |
| 24.8 | 3.59 | m | 19.3 |
| 25.4 | 3.51 | w | 7 |
| 26.2 | 3.40 | S | 60.6 |
| 26.6 | 3.34 | m | 22.2 |
| 27.1 | 3.29 | m | 29.7 |
| 27.4 | 3.25 | w | 10.2 |
| 28.0 | 3.19 | w | 8.9 |
| 28.4 | 3.14 | m | 26 |
| 28.7 | 3.11 | w | 12.8 |
| 29.7 | 3.00 | w | 7.7 |
| 29.9 | 2.98 | w | 8.5 |

### Methods of Use

### Disease Indications

The R-ketamine, salts, solid forms, and salt forms thereof of the present disclosure are useful in preparing a medicament for the prevention and/or treatment of various diseases or conditions.

The R-ketamine, salts, solid forms, and salt forms thereof herein are useful as a neuroprotective, preventive, or therapeutic agent for diseases or conditions associated with glutamatergic transmission, in particular, glutamatergic neurotransmission via an N-methyl-D-aspartate (hereinafter abbreviated as NMDA) receptor. Growing evidence suggests that abnormal glutamatergic transmission, e.g., by the NMDA receptor, is associated with the pathophysiology of mood disorders, and that the NMDA receptor also plays key roles in neurobiology.

The R-ketamine, salts, solid forms, and salt forms thereof herein are useful as a neuroprotective, preventive, or therapeutic agent for cognitive dysfunctions such as mood disorders, bipolar disorder, major depressive disorder, general anxiety disorder, panic disorder, obsessive compulsive disorder (OCD), post-traumatic stress disorder (PTSD), attention deficit hyperactivity disorder (ADHD), autism spectrum disorder (ASD), eating disorders, and substance use disorders (drug dependency).

The R-ketamine, salts, solid forms, and salt forms thereof are also useful treating neurodegenerative diseases and a number of medical diseases, including, but not limited to, cardiovascular diseases, cancer (also referred to as malignant tumors), inflammatory diseases, bone diseases and the like). Exemplary inflammatory and bone diseases include ulcerative colitis, Crohn's disease, rheumatoid arthritis, ankylosing spondylitis, insulin-dependent diabetes, Addison's disease, Goodpasture syndrome, IgA nephropathy, interstitial nephritis, Sjögren's syndrome, autoimmune pancreatitis, psoriasis, atopic dermatitis, pneumonia, chronic bronchitis, bronchial asthma, systemic lupus erythematosus (SLE), scleroderma, or delirium, and the bone disease is osteoporosis, osteolytic bone metastasis, and Paget's disease of bone. Since inflammatory diseases are chronic diseases that progress over a long period of time (years), beginning treatment early may prevent the progression of symptoms. Furthermore, by administering the R-ketamine, salts, solid forms, and salt forms thereof to patients having a genetic background that is potentially susceptible to inflammatory diseases before they exhibit symptoms, the R-ketamine, salts, solid forms, and salt forms thereof may also be used to prevent the onset of the inflammatory or bone disease.

The R-ketamine, salts, solid forms, and salt forms thereof are also useful in treating neurodegenerative diseases, such as Parkinson's disease, Alzheimer's disease, Lewy body dementia and the like. In some embodiments, the R-ketamine, salts, solid forms, and salt forms thereof treats one or more symptoms of the neurodegenerative disease.

The R-ketamine, salts, solid forms, and salt forms thereof are also useful in treating neurodevelopmental conditions or disorders, for example childhood or fetal neurodevelopmental disorders. Exemplary neurodevelopmental disorders include schizophrenia, autism spectrum disorder, attention-deficit/hyperactivity disorder, autism spectrum disorder and other learning disorders. Symptoms include cognitive impairment, such as impaired attention, reduced linguistic fluidity, reduced learning and retention of linguistic information, reduced processing speed, reduced declarative memory, impaired working memory, a reduced executive function, or a combination thereof. In some embodiments, the R-ketamine, salts, solid forms, and salt forms thereof treats one or more symptoms of a neurodevelopmental disorder. The R-ketamine, salts, solid forms, and salt forms thereof herein are useful as a neuroprotective, preventive, or therapeutic agent for diseases or conditions associated with brain dopamine loss. The R-ketamine solid forms, salts, and salt forms described herein may be used for preventing or treating diseases or conditions associated with a decrease of dopamine transporters (DAT). In particular, examples of dysfunctions or conditions of the brain dopamine nervous system include substance use disorders (drug dependency) known in abusers of stimulant drugs or cocaine, and R-ketamine is considered to be effective as a preventive or therapeutic drug for substance use disorders (drug dependency). It has been reported that dopamine transporter (DAT) is decreased in the brains of cocaine abusers and stimulant drug (methamphetamine) users, and it has been pointed out that the decrease in DAT is associated with cognitive dysfunction. In addition, the decrease in DAT in substance use disorder patients is similar to the decrease in DAT seen in some neurodegenerative diseases, for example Parkinson's disease patients.

In addition, the R-ketamine solid forms, salts, and salt forms thereof described herein may be involved in the signal mediated by the brain-derived neurotrophic factor BDNF which leads to it having a neuroprotective effect. Due to these neuroprotective effects, the R-ketamine solid forms, salts, and salt forms thereof described herein may be used not only for the prevention or treatment of depressive symptoms and neurodegenerative diseases, but also for the prevention or treatment of cognitive dysfunctions described above as the description of the term “cognitive dysfunction.”

By using the R-ketamine solid forms, salts, and salt forms thereof described herein as a preventive or therapeutic agent for a neurodevelopmental disorder, neurodegenerative disease, inflammatory or bone disease, or a cognitive dysfunction, it is possible to prevent the onset of the disease or disorder and to exert therapeutic effects such as alleviating and improving symptoms in patients suffering from a neurodevelopmental disorder, neurodegenerative disease, inflammatory or bone disease, or cognitive dysfunction. Since many of the disorders, diseases and dysfunctions described herein progress over a long period (measured in years), early treatment may also prevent the progression of, or reduce the severity of symptoms.

In addition, it may also be used for a patient with a genetic background potentially predisposing him or her to a neurodevelopmental disorder, neurodegenerative disease, inflammatory or bone disease, or a cognitive dysfunction to prevent the onset of the disorder, disease or dysfunction by administering it before the symptoms are exhibited.

In the present disclosure, the R-ketamine solid forms, salts, and salt forms thereof described herein thereof is used for the prevention or treatment of a neurodevelopmental disorder, neurodegenerative disease, inflammatory or bone disease, or a cognitive dysfunction, the administration of the drug is scheduled to be a long period of time.

Therefore, by using the R-ketamine solid forms, salts, and salt forms thereof described herein, it is possible to administer the drug for a long period of time without exerting side effects, so the R-ketamine solid forms, salts, and salt forms thereof described herein may be used as a preventive or therapeutic agent for a neurodevelopmental disorder, neurodegenerative disease, inflammatory or bone disease, or a cognitive dysfunction.

The R-ketamine solid forms, salts, and salt forms thereof described herein are useful as a preventative agent for a neurodevelopmental disorder, neurodegenerative disease, inflammatory or bone disease, or a cognitive dysfunction by preventing the onset of the disorder, disease or dysfunction, or as an agent for preventing the progression of the symptoms of the disorder, disease or dysfunction. In addition, the therapeutic agent may have a therapeutic effect of preventing the progression of the symptoms, and alleviating or improving the symptoms.

The R-ketamine solid forms, salts, and salt forms thereof of the present disclosure are useful in preparing a medicament for the prevention and/or treatment of one or more symptoms associated with any of the disorder, disease or dysfunctions described herein, for example depressive symptoms. In some embodiments, depressive symptoms include but are not limited a lack of interest in activities, changes in sleep pattern, changes in appetite, feelings of guilt, feelings of despair, lack of energy, trouble concentrating, stress, low mood or mood depression, impaired motivation, cognitive impairment, diminished ability to think, anxiety, insomnia, anhedonia and negative affect, anorexia, fatigue and suicidal thoughts. The R-ketamine solid forms, salts, and salt forms thereof of the present disclosure are useful in preparing a medicament for the prevention and/or treatment of depression. The R-ketamine solid forms, salts, and salt forms thereof of the present disclosure are useful in preparing a medicament for the prevention and/or treatment of treatment-resistant depression.

The R-ketamine solid forms, salts, and salt forms thereof of the present disclosure are useful in preparing a medicament for the prevention and/or treatment of a substance use disorder in a subject. In some embodiments, the substance use disorder comprises abuse of alcohol, marijuana, synthetic cannabinoids, opioids, stimulants, barbiturates, benzodiazepines, dextromethorphan (DXM), a sleep medication, khat, synthetic cathinones, cocaine, 3,4-methylenedioxymethamphetamine (MDMA), phencyclidine (PCP), lysergic acid diethylamide (LSD), psilocybin, an inhalant, Rohypnol, gamma-hydroxybutyric acid (GHB), N,N-Dimethyltryptamine (DMT), ayahuasea, mescaline, salvia, or nicotine.

The R-ketamine solid forms, salts, and salt forms thereof of the present disclosure are useful in preparing a medicament for the prevention and/or treatment of a substance use withdrawal symptom in a subject. In some embodiments, the substance use withdrawal symptom comprises a symptom of withdrawal from alcohol, marijuana, synthetic cannabinoids, opioids, stimulants, barbiturates, benzodiazepines, dextromethorphan (DXM), a sleep medication, khat, synthetic cathinones, cocaine, 3,4-methylenedioxymethamphetamine (MDMA), phencyclidine (PCP), lysergic acid diethylamide (LSD), psilocybin, an inhalant, Rohypnol, gamma-hydroxybutyric acid (GHB), N,N-Dimethyltryptamine (DMT), ayahuasca, mescaline, salvia, or nicotine.

Additional disorders, and symptoms thereof that can be treated by the R-ketamine solid forms, salts, and salt forms thereof of the present disclosure are described in WO 2015/037248 (PCT/JP2014/004730), WO 2019/213551 (PCT/US2019/030644), WO2019065900 (PCT/JP2018/036079), WO2019160057 (PCT/JP2019/005415), and WO2020138491 (PCT/JP2019/051605) which are incorporated by reference in their entireties.

### Routes of Administration

An R-ketamine solid form, salt or salt form described herein can be administered orally, nasally, intranasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

Dosage forms for the topical or transdermal administration of the R-ketamine solid forms, salts, and salt forms thereof described herein include powders, sprays, ointments, pastes, creams, lotions, gels, solutions, patches and inhalants. In one embodiment, the R-ketamine solid forms, salts, and salt forms thereof described herein may be mixed under sterile conditions with a pharmaceutically acceptable carrier, and with any preservatives, buffers or propellants that are required.

For administration by inhalation, the R-ketamine solid forms, salts, and salt forms thereof described herein of the disclosure can be delivered in the form of an aerosol spray from pressured container or dispenser, which can contain a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of, e.g., nasal sprays, rectal foam, or suppositories. For transdermal administration, an active compound can be formulated into an ointment, salve, gel, or cream as generally known in the art.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### Isolation of R-Ketamine

In some embodiments, any of the R-ketamine D-tartrate salts or polymorphic forms or R-ketamine L-tartrate salts or polymorphic forms described herein may be used in the isolation or preparation of R-ketamine. In some embodiments, any of the R-ketamine D-tartrate salts or polymorphic forms or R-ketamine L-tartrate salts or polymorphic forms described herein may be used in similar methods for isolating S-ketamine described in PCT/EP1997/002360 or PCT/BR2001/000075. In some embodiments, the isomer-pure R-ketamine D- or L-tartrate can be converted with hydrochloric acid into the corresponding hydrochloride.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort can be had to various other embodiments, modifications, and equivalents thereof which can suggest themselves to those skilled in the art without departing from the spirit of the present disclosure and/or scope of the appended claims.

| Abbreviation | Full name |
|---|---|
| Cyclodextrin | (2-Hydroxypropyl)-Beta-cyclodextrin |
| DCM | dichloromethane |
| dioxane | 1,4-dioxane |
| DMF | N,N-dimethylformamide |
| DMSO | dimethyl sulfoxide |
| DSC | differential scanning calorimetry |
| DVS | dynamic (water) vapor sorption |
| MEK | 2-butanone, methyl ethyl ketone |
| MTP | Microtiter plate |
| EtOH | ethanol (absolute) |
| EtOAc | Ethyl acetate |
| FT Raman | Fourier-transform Raman spectroscopy |
| MeCN/ACN | acetonitrile |
| MeOH | methanol |
| MIBK | methyl isobutyl ketone |
| MTP | microtiter plate |
| 1-PrOH | 1-propanol |
| 2-PrOH, IPA | 2-propanol |
| r.h. | relative humidity |
| r.t. | room temperature (22-25° C.) |
| TBME, MTBE | tert-butyl methyl ether |
| THF | tetrahydrofuran |
| T_{g} | glass transition temperature |
| TG-FTIR | thermogravimetry coupled to Fourier |
| | transform infrared spectroscopy |
| PTSA | para-toluenesulfonic acid; p- toluenesulfonic acid |
| PXRD, XPRD | powder X-ray diffraction |

### Experimental Procedures

### DSC

Differential scanning calorimetry was carried out with a TA Instruments Q2000 instrument (closed gold or aluminum sample pan, heating rates 5 or 10 or 20 K/min). The melting point is understood as the peak maximum.

### Dynamic Vapor Sorption

DVS measurements were performed with an SPS11-100n “Sorptions Prüfsystem” from ProUmid (formerly “Projekt Messtechnik”), August-Nagel-Str. 23, 89079 Ulm (Germany). About 5-20 mg of sample were put into an aluminum sample pan. Humidity change rates of 500 per hour were used. The applied measurement program is described as follows:

The sample was placed on an aluminum or platinum holder on top of a microbalance and allowed to equilibrate at 50% RH before starting the pre-defined humidity programs:
(1) 2 h at 50% RH
(2) 50→0% RH (5%/h); 5 h at 0% RH
(3) 0→95% RH (5%/h); 5 h at 95% RH
(4) 95→0% RH (5%/h); 5 h at 0% RH
(5) 0→95% RH (5%/h); 5 h at 95% RH
(6) 95→50% RH (5%/h); 2 h at 50% RH

### Classification of Hygroscopicity

The hygroscopicity was classified based on the mass gain at 85% RH relative to the initial mass as follows: deliquescent (sufficient water adsorbed to form a liquid), very hygroscopic (mass increase of ≥15%), hygroscopic (mass increase <15% but ≥2%), slightly hygroscopic (mass increase <2% but ≥0.2%), or non-hygroscopic (mass increase <0.2%).

### ¹H-NMR

Bruker DPX300 spectrometer; proton frequency of 300.13 MHz; 30° excitation pulse; recycle delay of 1 s; accumulation of 16 scans; deuterated DMSO as the solvent; solvent peak used for referencing; chemical shifts reported on the TMS scale.

### Powder X-Ray Diffraction

Stoe Stadi P equipped with a Mythen1K Detector; Cu-Kα1 radiation; standard measurement conditions: transmission; 40 kV and 40 mA tube power; curved Ge monochromator; 0.02°2θ step size, 48 s step time, 1.5-50.5°2θ scanning range; detector mode: step scan; 1 °2θ detector step; standard sample preparation: 10 to 20 mg sample was placed between two acetate foils; sample holder: Stoe transmission sample holder; the sample was rotated during the measurement. All sample preparation and measurement was done in an ambient air atmosphere.

### HPLC

Device from Agilent, Series 1100 (with Agilent 1260 Infinity degasser) with Chromeleon Version 6.8 software. Generic HPLC method used in this study is shown by the following parameters:

| Parameter | Description |
|---|---|
| column | Waters, XTerra MS C18, |
| | 100 × 4.6 mm, 5 μm (FK-CC01H) |
| sample dilution solvent | ACN |
| mobile phase A | Water + 25% NH3 1000/4 |
| mobile phase B | ACN |
| gradient | 0.0 min - 5% B |
| | 20.0 min - 95% B |
| | 20.1 min- 5% B |
| | 25 min - 5% B |
| flow | 1.0 mL min⁻¹ |
| injection volume | 10 μL |
| detection | 220 nm |
| column temperature | 25° C. |
| run time | 25 min |
| max. pressure | 79 bar |

HPLC method used to purify the R-ketamine fumarate salt Form A, the R-ketamine oxalate salt, and the R-ketamine saccharin salt

| Parameter | Description |
|---|---|
| column | Waters, XTerra MS C18, |
| | 100 × 4.6 mm, 5 μm (FK-CC01H) |
| sample dilution solvent | ACN/water 1:1 |
| mobile phase A | Water + 0.4% NH3 |
| mobile phase B | ACN |
| gradient | 0.0 min - 5% B |
| | 20.0 min - 95% B |
| | 20.5 min - 5% B |
| | 25 min - 5% B |
| flow | 1.0 mL min−1 |
| injection volume | 10 μL |
| detection | 220 nm |
| column temperature | 25° C. |
| run time | 25 min |
| max. pressure | 79 bar |

### Raman Spectroscopy

FT-Raman spectra were recorded on a Bruker MultiRAM FT-Raman system with a near infrared Nd:YAG laser operating at 1064 nm and a liquid nitrogen-cooled germanium detector. 64 scans with a resolution of 2 cm⁻¹ were accumulated in the range from 3500 to −50 cm⁻¹; however, only data above 100 cm⁻¹ are evaluated due to filter cutoff effects. Nominal laser powers are typically 100 or 300 mW.

### TG-FTIR

Thermogravimetric measurements were carried out with a Netzsch Thermo-Microbalance TG 209 coupled to a Bruker FTIR Spectrometer Vector 22 (sample pans with a pinhole, N2 atmosphere, heating rate 10° C./min).

### Example 1. Production of the R-Ketamine Free Base

The starting material was received as an R-ketamine hydrochloride salt. The first step was to produce the free base form from the hydrochloride salt. This HCl salt can be easily converted to the free base by neutralization with sodium bicarbonate solution and then extracted with dichloromethane. The resulting free base was characterized, and then used as starting material for the salt formation.

The R-ketamine hydrochloride salt was converted to the free base by neutralization with a sodium bicarbonate solution and then extracted with dichloromethane, 1.0 M sodium bicarbonate solution (100 ml) was slowly added to the aqueous R-ketamine hydrochloride solution (3.02 grams dissolved in 25 mL water, initial pH of 4) under stirring until the pH of the solution was around 9. Precipitation and foaming were observed immediately following the addition of the base. Stirring was continued overnight, and then the free base form of R-ketamine was extracted with dichloromethane (2×50 mL). The organic phase was separated, and the solvent was eliminated by evaporation in a rotatory evaporator. The resulting white solid was vacuum dried (<15 mbar, r.t.) in a round bottom flask over the weekend. After three days drying, 2.52 g of material was recovered (yield of 83%). The obtained material was characterized by PXRD (FIG. 1), TG-FTIR, FT-Raman (FIGS. 3 and 4), ¹H-NMR (FIG. 6), and DSC (FIG. 75). PXRD pattern is crystalline. DSC measurement revealed a melting peak at 121° C., with an associated enthalpy of about 110 J/g.

### Second Experiment for Producing the Free Base

R-ketamine HCL (3.3344 g) was dissolved in 25 mL of water under stirring at r.t. The pH was measured with pH-paper to be about 4. Sodium bicarbonate (8.399 g) was dissolved in water under sonication treatment in a 100 mL flask. A 1M aqueous sodium bicarbonate solution was obtained (pH 9) and 25 mL of the sodium bicarbonate solution was slowly added to the R-ketamine solution. Precipitation was observed as well as foaming. Water (25 mL) was added and pH was measured to be 7. The rest of the sodium bicarbonate solution was added: foaming was observed and 15 mL of water was added. The mixture was stirred at r.t. After overnight stirring, a solution was observed with some suspended material (flakes). Extraction was conducted with 2×50 mL of dichloromethane. The organic part was recovered in a round bottom flask for solvent evaporation with the rotary evaporator. The obtained residue was further dried under vacuum (<10 mbar, r.t.). After four days drying, 2.905 g of material was recovered.

Approximate solubilities were determined for the R-ketamine free base. These values were determined by adding small aliquots of solvent or solvent mixture to ˜10 mg of solid and shaking/sonicating for a short period of time at ambient conditions. These values are only approximations.

| Solvent System | Solubility [mg/ml] |
|---|---|
| 20% (2-hydroxypropyl)-beta-cyclodextrin in | 1.4 < S < 1.7 |
| water* | |
| Acetone | 111 < S < 222 |
| Acetonitrile | 89 < S < 133 |
| Ethanol | 62 < S < 83 |
| Ethyl acetate | 115 < S < 230 |
| Heptane | 5 < S < 9 |
| Methanol | 120 < S < 240 |
| 1-propanol | 60 < S < 79 |
| 2-propanol | 37 < S < 44 |
| THF | S > 288 |
| Water | S < 1 |

*The solubility of the HCl R-ketamine salt was over 100 mg/mL in aqueous 20% (2-hydroxypropyl)-beta-cyclodextrin solution.

### Scale-Up Experiment

R-ketamine hydrocholoride salt (10.0693 g) was dissolved in 85 mL of water under stirring at r.t. The pH was measured with pH-paper to be about 4-5. 29.4 g of sodium bicarbonate was dissolved in 350 mL of water under stirring at r.t. A 1M aqueous sodium bicarbonate solution was obtained (pH 9). 100 mL of the sodium bicarbonate solution was slowly added to the ketamine solution. Precipitation was observed as well as foaming; pH was of about 8. 100 mL of water was added and pH was measured to be 8. The rest of the sodium bicarbonate solution was added and a white good stirrable suspension was obtained (less foam was observed). 50 mL of water was added and pH was measured to be of about 8-9. The suspension was further stirred at r.t. After overnight stirring, a solution was observed with some suspended material (flakes) and foam. Extraction was conducted with 2×75 mL of dichloromethane. The organic part was recovered in a round bottom flask for solvent evaporation with the rotary evaporator. The obtained residue was further dried under vacuum (<10 mbar, r.t.). After two days drying, 8.9 g of material was recovered. PXRD pattern is crystalline and corresponds to the R-Ketamine free drug. 0.21% of water loss from 25 to 115° C. 0.22% DCM loss from 115° C. to 135° C. Decomposition is observed at higher temperature.

### Second Scale-Up Experiment

R-ketamine hydrocholoride salt (30.00 g) was dissolved in 255 mL of millipore water under stirring at r.t. The pH was measured with pH-paper to be about 4-5. 88.2 g of sodium bicarbonate was dissolved in 1050 mL of water under stirring at r.t. A 1 M aqueous sodium bicarbonate solution was produced (pH ˜9). 300 mL of the sodium bicarbonate solution was slowly added (10 mL-step) to the ketamine solution. Precipitation was observed as well as foaming. 100 mL of water was added and pH was about 8. The remaining sodium bicarbonate solution was added (20 mL-step) and a white suspension was obtained (less foam was observed). 200 mL of water was added and pH was measured to be about 8-9. The suspension was further stirred at r.t. After overnight stirring, a solution was observed with some suspended material and foam. Extraction was conducted with 3×750 mL of dichloromethane. The organic part was recovered in a round bottom flask for solvent evaporation with the rotary evaporator. The obtained residue was further dried under vacuum (<10 mbar, r.t.). After three days of drying, 25 g of material was recovered. PXRD pattern is crystalline and corresponds to R-ketamine free base. 0.53% of mass loss from 25° C. to 210° C. NMR spectrum consistent with ketamine structure. Residual DCM visible at 5.75 ppm (0.008 eq).

### Example 2. Formation and Evaluation of Various R-Ketamine Salts

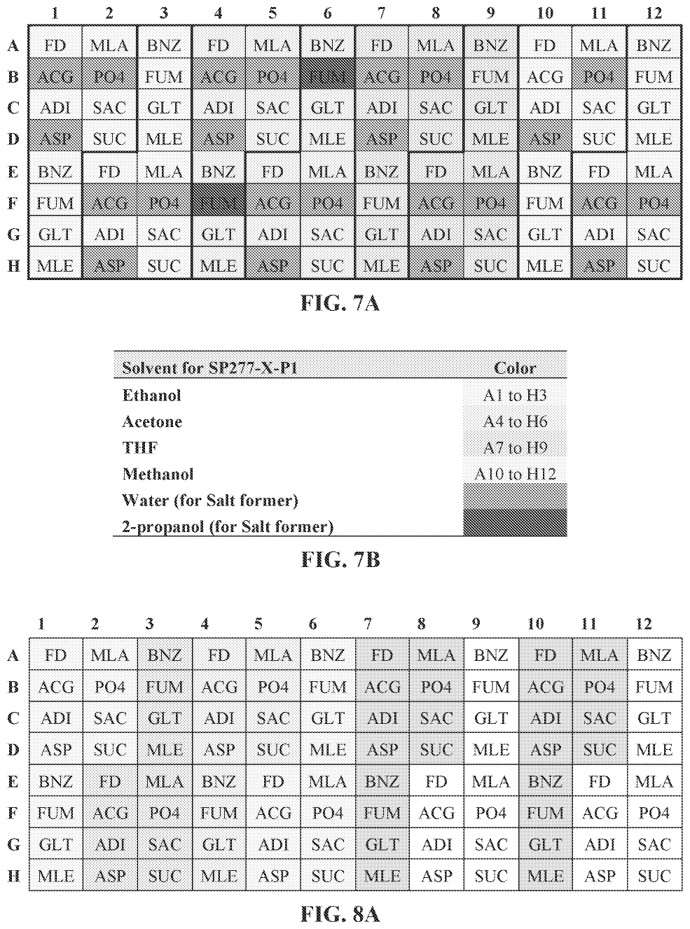

Salt crystallizations were carried out with eleven different salt/co-crystal formers, and the free base was used alone in blank experiments. The experiments were carried out in a quartz 96-microtiter plate according to the methods described in U.S. Pat. No. 7,504,071. The first step of the HTS was an evaporative plate, referred to as Phase 1 in this study. The experiments were carried out by adding a 0.05 M free base stock solution (Phase 1) to each well followed by the addition of the salt former stock solutions specified in Table 14 and according to the plate layout in FIG. 7A. The solvents were evaporated from each well under nitrogen flow at room temperature. The solid residues obtained in the wells were investigated by Raman microscopy.

To each well of a 96 well quartz plate was added 100 μL of a 0.05 M free base solution:
A 1-H3 in ethanol
A4-H6 in acetone
A7-H9 in THF
A10-1H12 in methanol

Then was added 100 μL of a 0.05 M solution of salt former in the corresponded solvent. N-Acetylglycine, aspartic acid and phosphoric acid were always added in water. Fumaric acid cannot be dissolved in acetone and was also added in 2-propanol. The solvent was evaporated at room temperature with nitrogen flow (300 mL/min for 2 days and then 4 days with 500 mL/min).

TABLE 14
List of stock solutions used for salt crystallization.

| Salt/Co-Crystal Former | Code | CAS-No | Formula | M |
|---|---|---|---|---|
| N-Acetylglycine | ACG | 543-24-8 | C₄H₇NO₃ | 117.1 |
| Adipic acid | ADI | 124-04-9 | C₆H₁₀O₄ | 146.14 |
| Aspartic acid | ASP | 56-84-8 | C₄H₇NO₄ | 133.1 |
| Benzoic acid | BNZ | 65-85-0 | C₇H₆O₂ | 122.12 |
| Fumaric acid | FUM | 110-17-8 | C₄H₄O₄ | 116.07 |
| Glutaric acid | GLT | 110-94-1 | C₅H₈O₄ | 132.12 |
| Maleic acid | MLE | 110-16-7 | C₄H₄O₄ | 116.07 |
| L-Malic acid | MLA | 97-67-6 | C₄H₆O₅ | 134.09 |
| Phosphoric acid | PO4 | 7664-38-2 | H₃O₄P | 98 |
| Saccharin | SAC | 81-07-2 | C₇H₅NO₃S | 183.18 |
| Succinic acid | SUC | 110-15-6 | C₄H₆O₄ | 118.09 |

Results from Phase 1: After solvent evaporation, visual investigation of the microtiter plate revealed several positions that contained solid and possibly crystalline material. Raman microscopy investigation was conducted for the whole plate. Crystalline material was examined and two to seven locations for Raman spectroscopy analysis were selected in each well.

The blank experiments with the free base alone were first examined, in order to see if another crystalline form or possibly a solvate of the free base have been formed. A new spectrum was obtained from a location in well A7. All the other measurements correspond to the free base reference apart from some cases where the band at 1700 cm-1 disappeared. This new Raman spectrum contains the bands of the free base reference; however, it presents an intense band at 1050 cm-1 and two new bands around 700 cm-1. Another polymorph of the free base could be formed during the experiment with THF. A THF-solvate of the free base can be another possible hypothesis.

TABLE 15
Results from the evaporation experiments of Phase 1. The table contains the
salt code, the well numbers with remarks and reference to the Raman data.

| Salt Code | Well # | Remarks |
|---|---|---|
| ADI | C1c | Raman spectra correspond to a mixture of the free base and |
| | | adipic acid but with shifts for the ADI bands. Additional band at |
| | | 734 cm⁻¹ |
| | | (FIG. 41). Observed in one well. |
| | | Crystalline material. |
| ADI | G2a, G2b, G8, G11 | Raman spectra correspond to a mixture of the free base and |
| | | adipic acid but with shifts for the ADI bands. |
| | | Observed in several wells. |
| | | Crystalline material. |
| BZN | E1, E4, A6, A9, A12 | Spectra correspond to mixture free base and benzoic acid with |
| | | slight shifts. Some BZN bands are missing. |
| | | Possible crystalline material. |
| BZN | E7 | Small amount of free base visible but some bands are missing |
| | | and new bands are observed. BZN is not observed. |
| | | Crystalline material |
| FUM | F1, F4, F7, F10, B6, | Raman spectra correspond to mixture free base and fumaric acid. |
| | B9, B12 | Some free base bands are missing and additional bands are |
| | | observed. |
| | | Crystalline material |
| | | Good lead |
| SAC | C2, C5, C8, G3, G6, | New spectra. Small amount of the free base, saccharin bands |
| | G9 | present but with shifts. Several new bands. |
| | | Crystalline material |
| | | Very good lead |
| SUC | D2, D8, H3, H9 | Spectra correspond to free base with shift and additional bands. |
| | | Succinic acid visible |
| | | Crystalline material |
| | | Good lead |

### Example 3: Slurry Crystallization

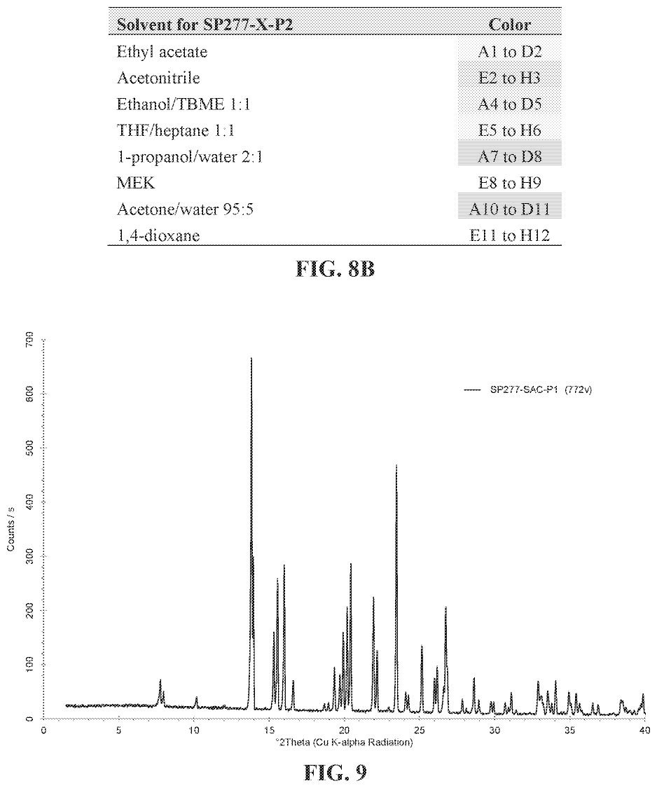

In a second crystallization experiment, henceforth referred to as Phase 2, eight solvent systems were selected for slurry equilibrations. 100 μL of the selected solvent system was added to the residue of Phase 1 (Example 2) for slurry equilibrations according to the layout in FIG. 8A (domain in color) and FIG. 8B. After two days shaking at room temperature, many of the wells contained solutions; however, in some wells, solid material was observed on the bottom of the well, and is probably the rest of the evaporative residues which could not be dissolved or slurried by the solvent systems. Afterwards, the solvents were evaporated under a nitrogen flow (200 mL/min) at room temperature over the weekend, and the obtained solid residues were investigated by polarized light microscopy. The positions containing crystalline material were investigated by Raman microscopy.

Results: After two days equilibration and solvent evaporation, the Phase 2 plate was investigated by light microscopy and crystalline material was observed in the majority of the wells. Raman investigation was conducted on the wells containing crystalline materials.

As for the previous evaporation plate, no salt formation was observed with N-acetylglycine, L-asparagine, glutaric acid, L-malic acid, maleic acid and phosphoric acid. Furthermore, no salt formation was observed with adipic acid in this case.

Salt formation was found at least with benzoic acid, fumaric acid, saccharin and succinic acid, and the obtained Raman spectra were the same as observed in the previous Phase 1 plate. Table 16 summarizes the obtained results for the plate from Phase 2.

TABLE 16
Results from the evaporation experiments. The table contains the salt
code, the well numbers and remarks and reference to the Raman data

| Salt Code | Well # | Remarks |
|---|---|---|
| BZN | E1, E4, E7, E10, | Spectra correspond to mixture free base and benzoic acid |
| | A3b, A6 A12 | with shifts. Some BZN bands are missing. |
| | | Possible crystalline material. |
| BZN | A3 | Free base visible but some bands are missing and new bands |
| | | are observed |
| | | Corresponds to well E7 from previous plate. |
| | | Crystalline material |
| FUM | F4, F1, B3, B9 | Spectra correspond to mixture free base and fumaric acid. |
| | | Some FD bands are missing and additional bands are |
| | | observed. |
| | | Corresponds to well F1 from previous plate. |
| | | Crystalline material |
| | | Good lead |
| FUM | B3, B9, F7 | Spectra similar to the previous lead with FUM but some |
| | | bands are missing. |
| | | Crystalline material |
| | | Good lead |
| SAC | G9, C2, C8, G3, | New spectra. Small amount of the free base, saccharin |
| | G6, G12 | bands are present with shifts. Several new bands. |
| | | Corresponds to well C8 from previous plate. |
| | | Crystalline material |
| | | Very good lead |
| SUC | H12, D5, H9 | Spectra corresponds to free base with shifts, and additional |
| | | bands (some bands corresponds to typical shift for the FD). |
| | | Corresponds to well D2 from previous plate. |
| | | Crystalline material |
| | | Poor/Intermediate lead |

During these salt formation experiments, several leads for potential salt formation were found with benzoic acid, fumaric acid, saccharin and succinic acid. Based on these results the saccharin salt was further scaled-up on a 100 g scale. The Raman investigation indicates a new crystalline compound and possibly a saccharin salt, but it should not be ruled out that another crystalline form of the free base could possibly be obtained.

### Example 4. R-Ketamine Saccharin Salt

R-ketamine free base (300.2 mg, 1.26 mmol) was dissolved in 10 mL of 2-propanol under stirring at room temperature. Short sonication treatment was used to dissolve the material. Saccharin 231.8 mg (1 eq, 1.26 mmol) was dissolved in 15 mL of 2-propanol at r.t. using sonication treatment. The solution with saccharin was slowly added to the free base solution under stirring at r.t. After the addition a clear solution was obtained but became cloudy after a couple of minutes and transformed to a fine suspension. After overnight stirring, a fine suspension was obtained and the vial was opened to allow solvent evaporation under stirring. After one day, ¼ of the solvent was evaporated and the suspension was filtered over fritted glass (porosity 4). The filter cake was dried 5 minutes on the filter with vacuum and 442 mg of powder was recovered (yield of 83%).

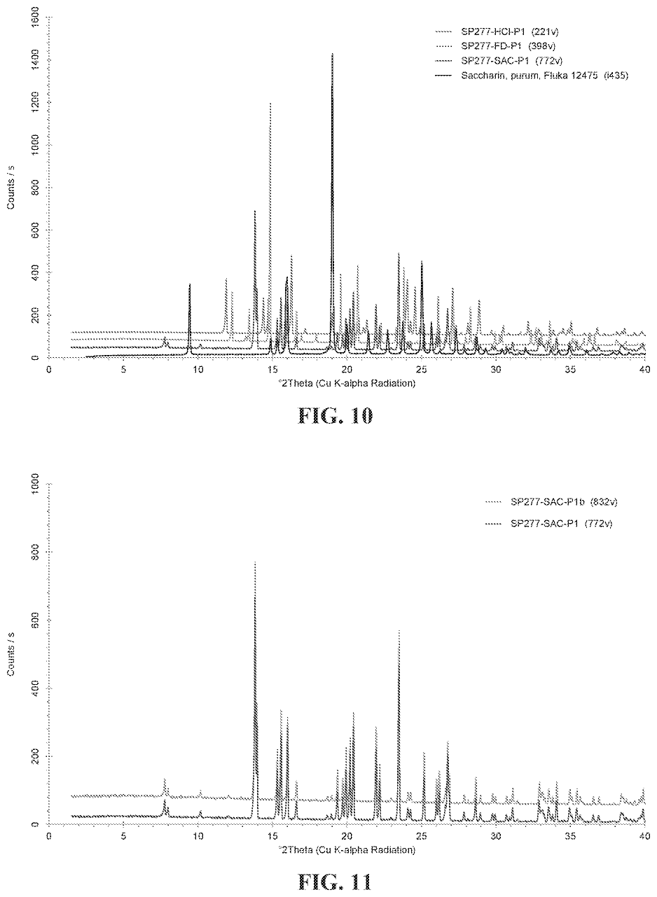

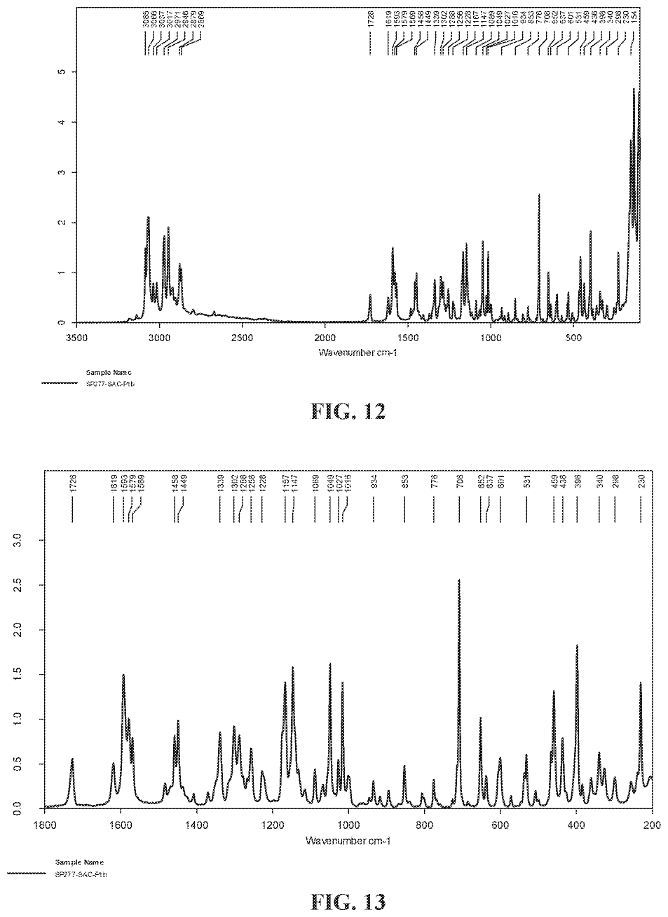

Salt formation was observed with saccharin during the salt/co-crystal experiments. Thus, an experiment was conducted attempting to produce a larger amount of the R-ketamine saccharin salt. The free base was dissolved in 2-propanol at room temperature. An equimolar amount of saccharin was also dissolved in 2-propanol, and this solution was slowly added to the free base solution under stirring at room temperature. A clear solution was observed after the addition and suspension formed after a couple of minutes. After two days stirring, the suspension was filtered, and the wet cake was analyzed by PXRD. A new crystalline PXRD pattern was obtained (FIG. 9), which is different from the PXRD patterns of the free base and saccharin (FIG. 10). The wet cake was dried at 5 mbar overnight at room temperature; the dried sample was submitted for PXRD investigation and no change in the PXRD pattern was observed (FIG. 11). The dried sample was used for further characterization.

An FT-Raman spectrum was recorded, and the obtained Raman spectrum was the same as obtained during the previous experiments (FIG. 14). Therefore, the initial results with saccharin could be successfully and reliably reproduced.

TG-FTIR shows 0.28% of mass loss up to 220° C., which corresponds to trace of water and 2-propanol (FIG. 15). Therefore, the obtained salt is an anhydrous form.

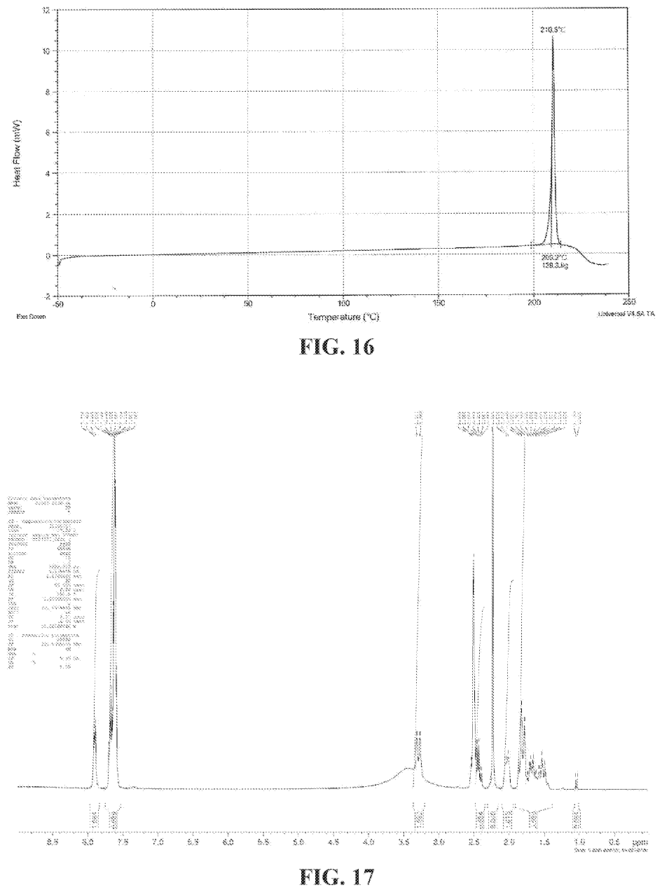

DSC measurement revealed a melting peak at 210.5° C., with an onset at 209.2° C. and an associated enthalpy of 139.3 J/g (FIG. 16). The melting peak of the hydrochloride salt was found at 270.5° C.

¹H-NMR measurement confirmed the one to one stoichiometry of the R-ketamine-saccharin salt; residual 2-propanol is also observed in the NMR spectrum (FIG. 17).

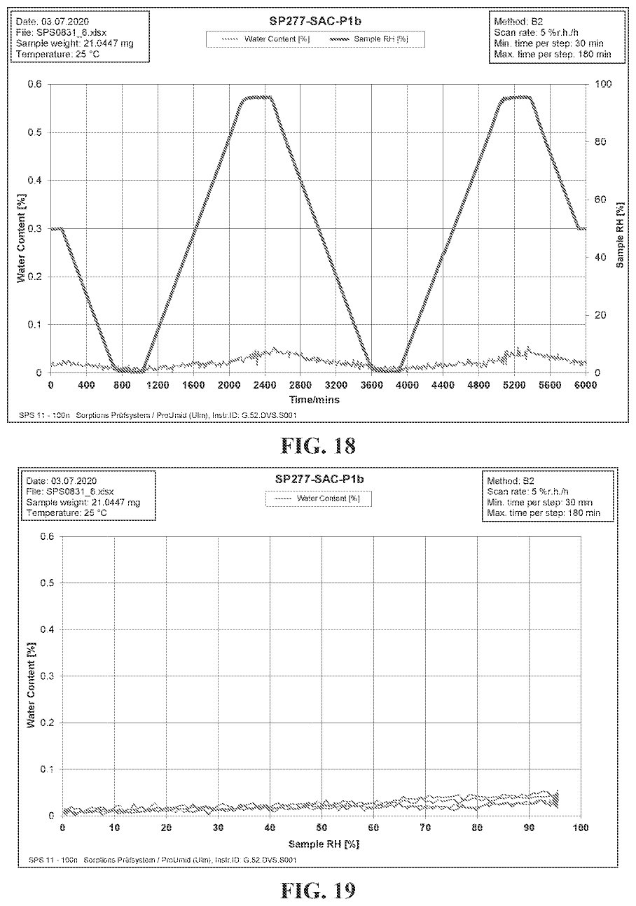

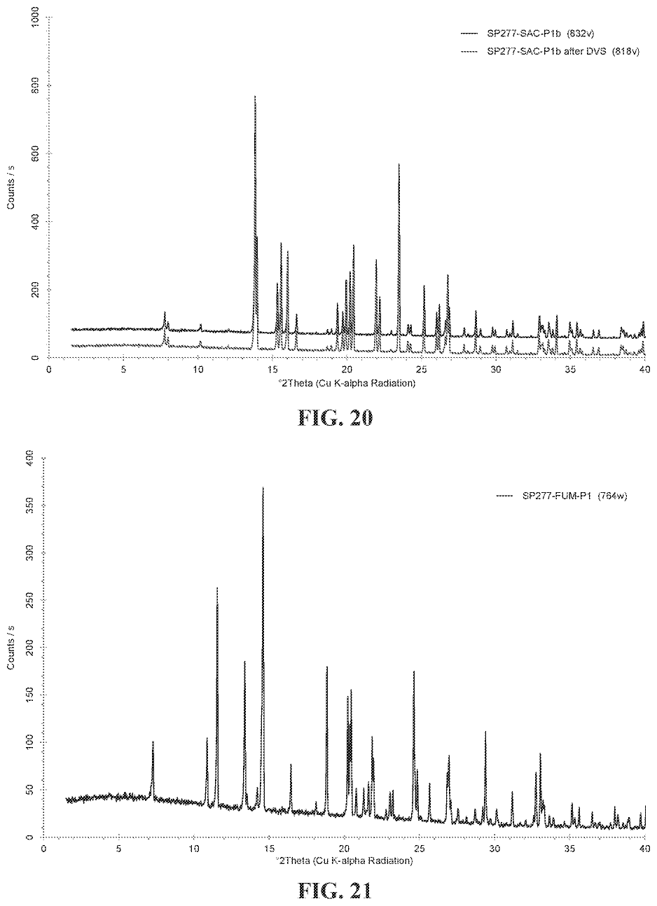

DVS measurement revealed a non-hygroscopic saccharin salt with less than 0.1% of water uptake upon storage at 95% relative humidity. The result from the DVS test is presented in FIG. 18 and FIG. 19. PXRD investigation was conducted on the sample after DVS and as expected, no change in the solid form was observed (FIG. 20).

The chemical identity of the R-ketamine saccharin salt was verified by elemental composition analysis using CHSNO contents determinations and TG-FTIR for the water and solvent content. The obtained results are summarized in Table 17. The obtained results fit very well with the theoretical content of a (1:1) saccharin salt.

TABLE 17
Result from CHNSO content analysis for R-ketamine saccharin salt
sample compared with the theoretical composition of a (1:1) solvent
and water-free salt with a molecular mass of 420.91 g/mol and the formula
C₂₀H₂₁N₂O₄ClS.

| | Experimental | Expected content for |
|---|---|---|
| | R-ketamine | a solvent and water-free |
| | saccharin | R-ketamine saccharin |
| Element | salt | salt |
| C | 56.9% | 57.1% |
| H | 4.9% | 5.0% |
| N | 6.5% | 6.7% |
| O | 15.2% | 15.2% |
| Cl | — | 8.4% |
| S | 7.5% | 7.6% |
| Water by TG-FTIR | 0.3% | 0% |

### Example 5. R-Ketamine Fumarate Salt Form A

R-ketamine free base (301.1 mg, 1.27 mmol) was dissolved in 4 mL of ethanol at r.t. with sonication treatment. Fumaric acid (73.5 mg, 0.63 mmol, 0.5 eq) was dissolved in 2 mL of ethanol at r.t. with sonication treatment. Then the fumaric solution was slowly added to the free base solution under stirring. After the addition a clear solution was obtained, and further stirring was conducted at r.t. with open vial. After 10 minutes, suspension started to form. After four hours, the suspension was filtered (centrifugal unit filter, PVDF, 0.22 μm, 5 min, 5000 rpm, r.t.). The obtained powder was dried at r.t., 5 mbar for 5 days.

Salt formation was observed with fumaric acid during the salt/co-crystal experiment. Therefore, an experiment was conducted in order to reproduce the R-ketamine fumarate salt.

In this experiment, the free base was dissolved in ethanol at room temperature. A half equivalent of fumaric acid was dissolved in ethanol and this solution was slowly added to the free base solution under stirring. After the addition a clear solution was obtained; a suspension started to form after ten minutes stirring at r.t. After four hours stirring, the obtained suspension was filtered, and the resulting powder was dried at r.t. with approx. 5 mbar. The obtained material was characterized by PXRD, FT-Raman, H-NMR and TG-FTIR.

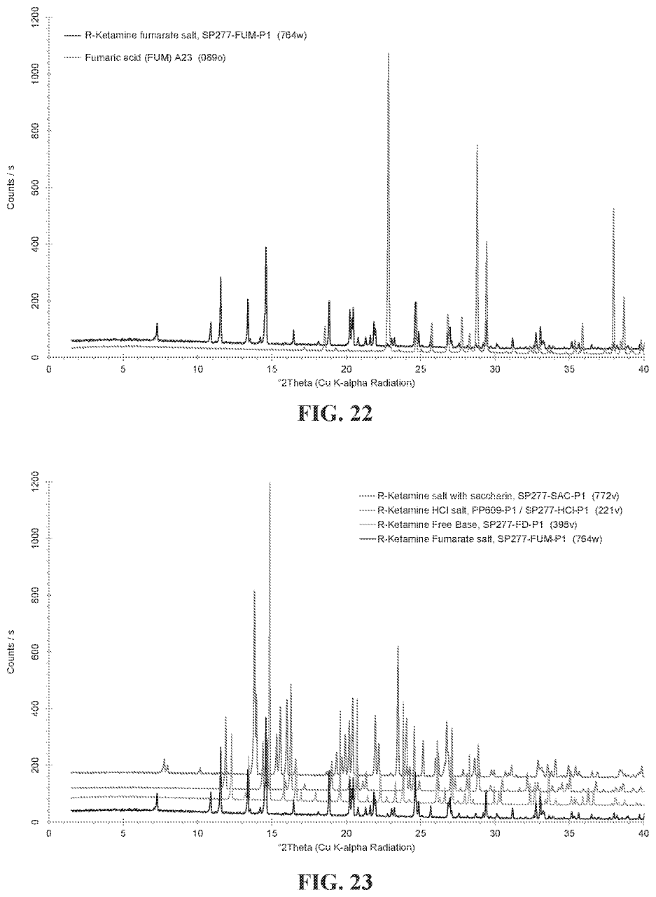

A new crystalline PXRD pattern was obtained for the fumarate salt sample (FIG. 21); this pattern is different from the fumaric acid reference (FIG. 22) and also from the PXRD patterns of the free base and the salts with saccharin and HCl (FIG. 23).

¹H-NMR was measured for the fumarate salt sample; 0.5 equivalent of fumaric acid was found and confirmed the free base to acid 2:1 ratio. The ¹H-NMR is depicted in FIG. 24.

FT-Raman was measured on the fumarate salt sample and the spectrum is depicted in FIG. 25 and FIG. 26. Additionally, FIG. 27 shows an overlay with the lead obtained during the HTS experiments; the spectra are different. Therefore, one can assume that the 1 to 1 salt was obtained during the HTS experiment instead of the 2:1 free base to fumaric acid salt.

TG-FTIR measurement revealed only trace of water with 0.03% mass loss from 25 to 150° C. Therefore, this sample of the R-ketamine fumarate salt Form A is a non-solvated form, essentially without any residual solvent. Furthermore, DSC analysis revealed a sharp melting peak at 144.2° C. with an associated enthalpy of 136.6 J/g (FIG. 28B)

The R-ketamine fumarate salt Form A was also measured by DVS; the obtained curves are depicted in FIG. 28C and FIG. 28D. This fumarate salt takes up to 6% of water upon storage at 95% relative humidity and the sample lost all its water when the humidity is scanned back to 0% r.h. PXRD investigation was conducted on the sample after DVS and no change in the crystalline form was observed FIG. 28E.

### Example 6. R-Ketamine Succinate Salt

R-ketamine free base (200.3 mg, 0.84 mmol) was dissolved in 5 mL of 2-propanol at r.t. Short sonication treatment was used to dissolve the material and 49.7 mg of succinic acid (0.5 eq, ˜0.42 mmol) was dissolved in 2 mL of 2-propanol at r.t. using sonication treatment. The solution with succinic acid was slowly added to the free base solution under stirring at r.t. After the addition a clear solution was obtained. Further stirring was conducted at r.t. with open vial. After 5 days, a solution was obtained with some material; further evaporation was conducted with nitrogen flow at r.t. After overnight evaporation, a glassy residue was obtained, and 1 mL of acetone was added. After vortex treatment, a solution was obtained, and further stirring was conducted at r.t. After 10 minutes a thick suspension was obtained, and additional 1 mL of acetone was added. A solution resulted and 2 mL of heptane was slowly added. A cloudy solution formed and transformed in a thick suspension. Filtration was conducted using centrifugal unit filter (PTFE, 0.22 m, 5 min, 5000 rpm, r.t.). The obtained filter cake was dried overnight at r.t., vacuum <5 mbar.

A possible crystalline salt was observed with succinic acid. Therefore, an experiment was conducted in order to see if this salt can be reproduced in larger scale, i.e., 200 mg. An R-ketamine succinate salt was obtained with 0.5 equivalent of succinic acid in acetone by precipitation with heptane (1:1 ratio) in the experiment. The obtained material was characterized by PXRD, FT-Raman, ¹H-NMR and TG-FTIR.

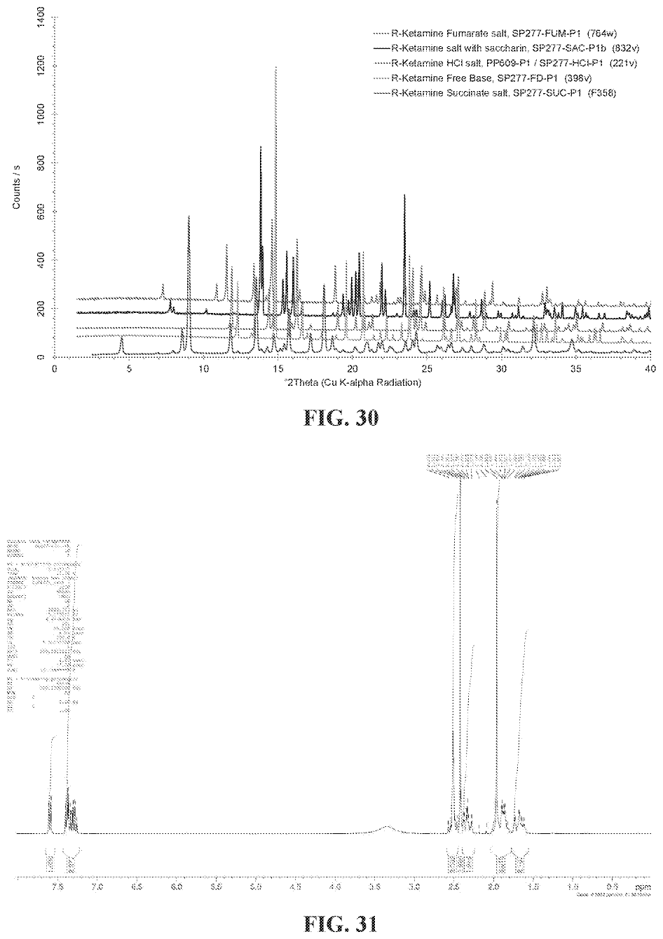

PXRD analysis revealed a new crystalline pattern (FIG. 29) which is different from the other crystalline forms (FIG. 30).

The ¹H-NMR spectrum (FIG. 31) of the R-ketamine succinate salt sample is consistent with the R-ketamine structure and one equivalent of succinic acid was found; this is somewhat surprising because the experiment was started with 0.5 equivalent of succinic acid.

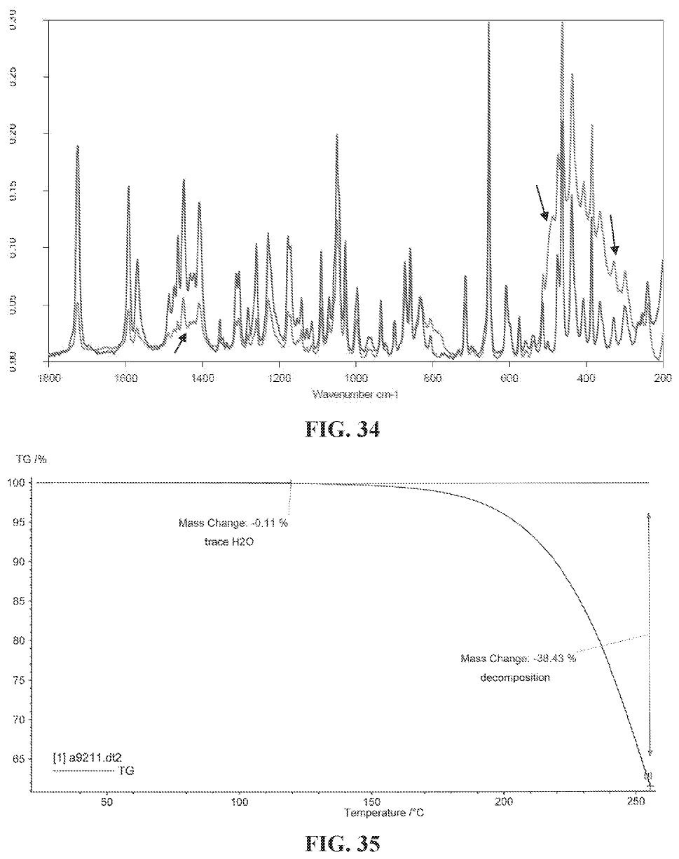

The FT-Raman spectrum of the succinate salt (FIG. 32 and FIG. 33) is the same as obtained during the screening experiments (FIG. 34). Therefore the 1:1 succinate salt obtained during screening experiments could be successfully and reliably reproduced.

TG-FTIR measurement revealed only trace of water with 0.11% mass loss from 25 to 120° C. and therefore a solvent-free salt was produced (FIG. 35).

### Example 7. R-Ketamine Sulfate Salt

R-ketamine free base (190.8 mg, 0.803 mmol) was dissolved in 2 mL of methanol at r.t. under stirring. Then 4.01 mL (0.5 eq, 0.402 mmol) of a sulfuric acid 0.1M aqueous solution was slowly added. After the addition a clear solution was obtained. After overnight stirring at r.t., a solution was still observed, and further stirring was conducted open vial. After four days, still a solution; further evaporation was conducted with nitrogen flow. A gel with glassy residue was obtained. 500 μL of methanol was added and the material was dissolved. 3 mL of TBME were slowly added but no precipitation was observed. The solution was further stirred, open vial. Dried material was obtained and 500 μL of TBME was added. A solution was obtained with sticky material on the side. 200 μL of ethanol was added and a suspension formed. After 4 hours of stirring, the suspension was filtered using centrifugal unit filter (PTFE, 0.22 μm, 5 min, 5000 rpm, r.t.). The obtained filter cake was dried overnight at r.t., vacuum <5 mbar.

Salt formation with sulfuric acid was tried in methanol. However, no precipitation was observed and the obtained solution was evaporated. TBME was added to the solid residue and sticky material formed. Ethanol was added and a suspension could be stirred at r.t. After filtration, the material was dried overnight under vacuum and then characterized. A second experiment was conducted but the resultant material was amorphous.

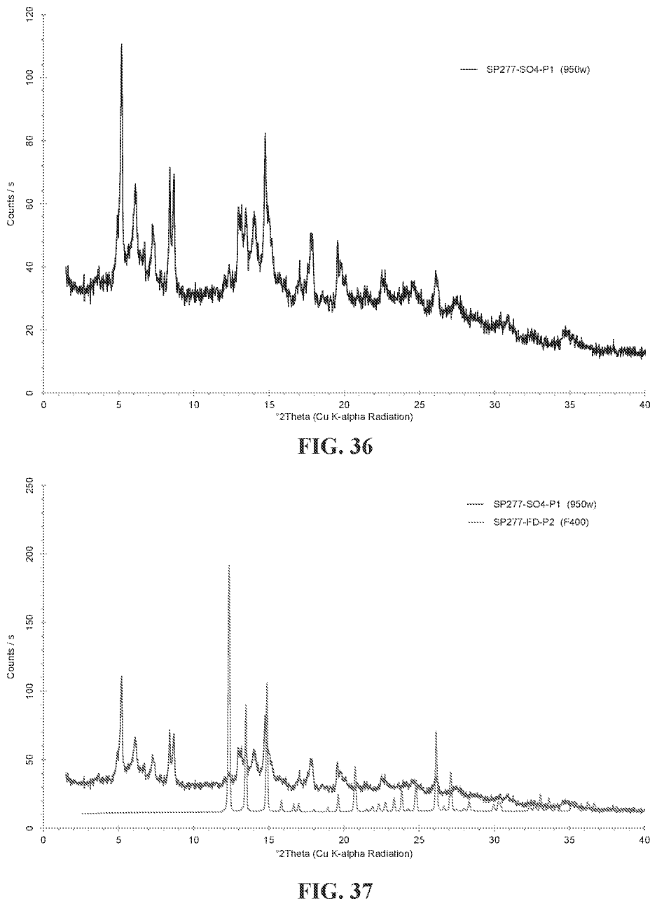

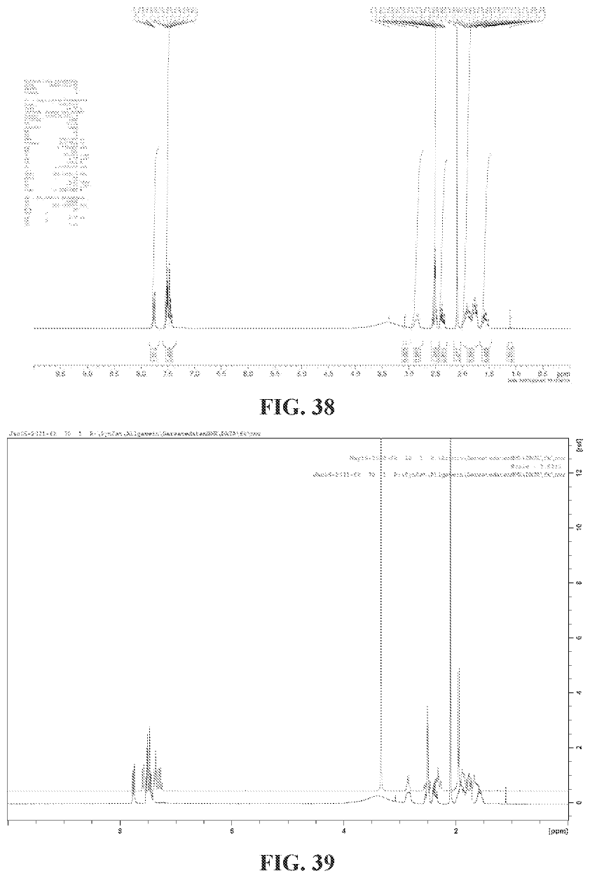

PXRD analysis on the dried material revealed a new PXRD pattern but with low crystallinity (FIG. 36). FIG. 37 shows an overlay with the free base starting material.

¹H-NMR was conducted on the sample (FIG. 38); the obtained spectrum is consistent with the R-ketamine structure. Furthermore, changes (shifts, multiplicity, etc.) are observed compared to the NMR spectrum of the starting material and suggest structural changes (FIG. 39). Therefore, salt formation can be considered. Further investigations on the sample, notably with elemental analysis, are being conducted in order to determine the equivalence in sulphur. Some residual TBME (<0.02 eq) is observed at 1.11 ppm and 3.08 ppm.

R-ketamine free base (296.9 mg, 1.25 mmol) was dissolved in 2 mL of acetone at r.t. under sonication treatment. Then 625 uL (0.5 eq, 0.63 mmol) of a sulphuric acid 2N (1M) aqueous solution was slowly added. After the addition a clear solution was obtained and further stirring was continued with open vial. A gel was obtained and 1 mL of 2-propanol was added. After sonication treatment, a solution was obtained. After two days equilibration, no precipitation was observed and 2 mL of heptane was slowly added. An emulsion with gel part was obtained and didn't crystallize after one day of stirring. The solvents were then evaporated under nitrogen flow and a white solid residue was obtained. 2 mL of TBME was added and a suspension formed. After stirring over a weekend, a thick suspension was observed. Filtration was conducted using a centrifugal unit filter (PTFE, 0.22 μm, 5 min, 5000 rpm, r.t.) and the reactor was flushed with the filtrate. The recovered filter cake was dried under vacuum (<5 mbar) at room temperature. After overnight drying, 293 mg of powder was recovered which resulted in an amorphous PXRD.

### Example 8. R-Ketamine D-Tartrate Salt Form A

R-ketamine free base (196.3 mg, 0.826 mmol) was dissolved in 2.5 mL of ethanol at r.t. under stirring and 62 mg of D-tartaric acid (0.413 mmol, 0.5 eq) was dissolved in 2 mL of ethanol at r.t. Then the tartaric solution was slowly added to the free base solution under stirring. After the addition a clear solution was obtained and further stirring was conducted at r.t. After overnight stirring at r.t., a solution was still observed and further stirring was conducted open vial. After four days, solid material was obtained on the side with a solution. The material was scratched with a spatula and the mixture was further stirred at r.t. After overnight stirring, a suspension formed. Filtration was conducted using centrifugal unit filter (PTFE, 0.22 μm, 5 min, 5000 rpm, r.t.). The obtained filter cake was dried overnight at r.t., vacuum <5 mbar.

Salt formation was conducted in ethanol with D-tartaric acid. The obtained dried material was first characterized with PXRD. A new and crystalline PXRD was obtained (FIG. 40). FIG. 41 shows an overlay of the new pattern with the free base starting material and D-tartaric acid.

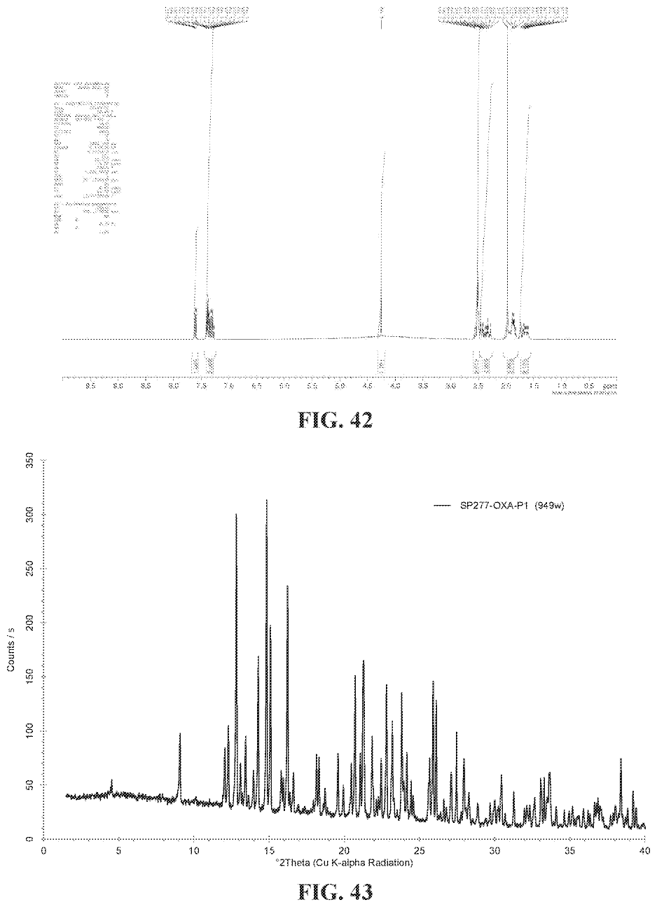

¹H-NMR on the R-ketamine D-tartrate salt sample revealed a spectrum which is consistent with the R-ketamine structure (FIG. 42). Some changes are observed compared to the spectrum of the starting material and confirmed structural change in the environment. Approximately 0.8 equivalent of tartaric acid was found at 4.25 ppm. A broad shoulder is observed under the tartaric peak and therefore the exact equivalence is difficult to predict but could be between 0.75 and 0.85.

The DSC thermogram of the R-ketamine D-tartrate salt Form A is presented in FIG. 53. DSC shows a fairly sharp melting endotherm with a peak maximum at 108° C. which is followed by two smaller signals. The current knowledge is not sufficient to provide further interpretation for the smaller signals. The event near 200° C. is possibly due to chemical degradation.

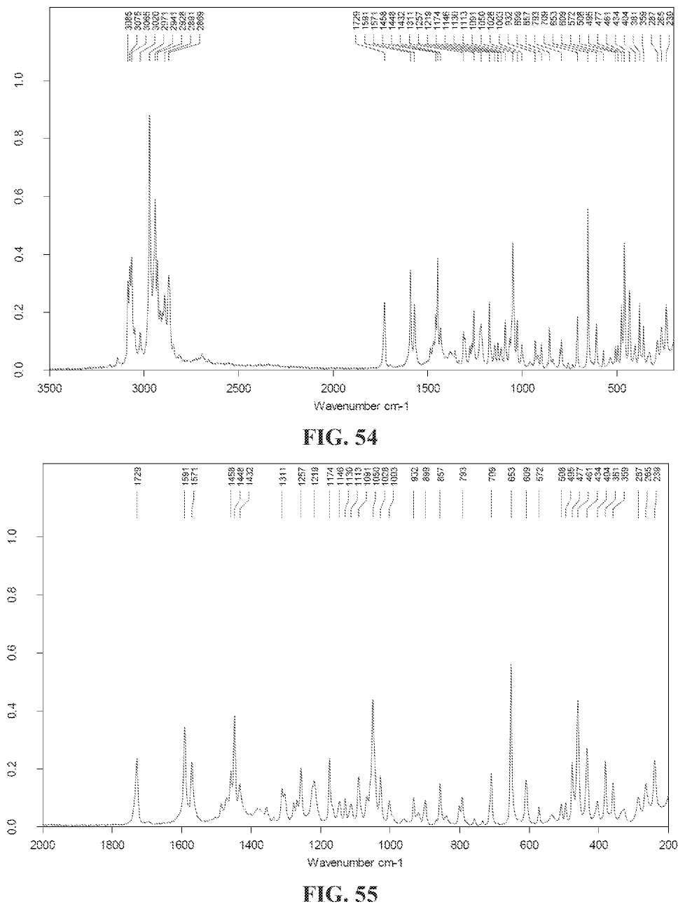

An FT-Raman spectrum was recorded for the R-ketamine D-tartrate salt Form A. FIG. 54 shows the full range of the spectrum and FIG. 55 shows the zoomed-in fingerprint region (from 200 cm-1 to 2000 cm-1).

Reanalysis of the material after 13 months of storage in a closed vial at ambient conditions gave a PXRD pattern corresponding to R-ketamine D-tartrate salt Form A. TG-FTIR shows a mass loss of 2.8%, which is essentially attributable to water (FIG. 76). The mass loss is associated with a rather clear step that begins at about 80° C. This step suggests a stoichiometric hydrate; however, the 2.8% loss would correspond to 0.6 waters per salt entity and a hemi-hydrate would contain 2.3% water. This seems unusual and a single crystal structure might be required to clarify the stoichiometry.

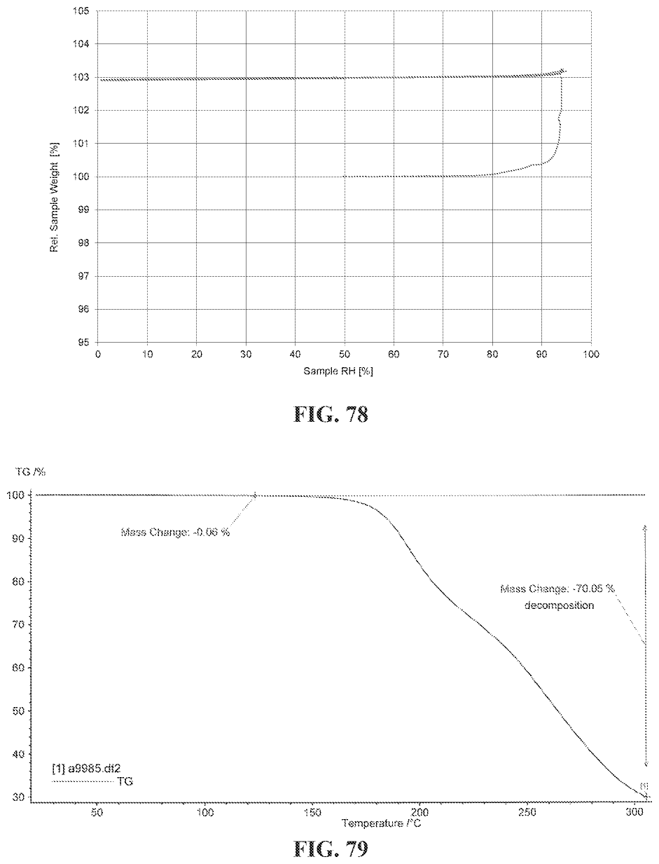

DVS measurement shows that above 70% relative humidity an additional water uptake of about 3% occurs. In course of the measurement, this water content does not change much anymore. The water sorption is irreversible under the test conditions. The result from the DVS test is presented in FIG. 77 and FIG. 78. The solid recovered after the DVS test was investigated using FT-Raman, which gave a mixture of R-ketamine D-tartrate salt Form D and free base.

### Second Experiment for Producing R-Ketamine D-Tartrate Salt Form A

R-ketamine free base (195.2 mg) was dissolved in 3.25 mL of ethanol, and 0.5 equivalents of D-tartaric acid was added in form of a solution in ethanol (1.25 mL). The solution was seeded with a few crystals of R-ketamine D-tartrate salt Form A. The solution was let to stir at r.t. for a day and fine particles were then observed. The suspension was let stir at r.t. for 3 more days but most of the crystalline material stuck to the glass wall of the vial. The material was resuspended and the open vial was kept under stirring at r.t. for 5 more days. Crystals were then obtained and examined by PXRD, which gave a mixture of R-ketamine D-tartrate salt Form A, D, and the crystalline free base form.

### Example 9. R-Ketamine Oxalate Salt

R-ketamine free base (180.7 mg, 0.76 mmol) was dissolved in 2 mL of acetonitrile at r.t. And 34.2 mg of oxalic acid (0.5 eq, 0.38 mmol) was dissolved in 1 mL of acetonitrile at r.t. The oxalic acid solution was slowly added to the free base solution under stirring. After the addition, no precipitation was observed and the solution was further stirred at r.t., open vial. A paste was obtained and 500 μL of TBME was added. Precipitation was observed. After four days of stirring at r.t., some material stuck to the side. 100 μL of ethanol was added and a nice suspension formed. After four hours of stirring, the suspension was filtered using centrifugal unit filter (PTFE, 0.22 μm, 5 min, 5000 rpm, r.t.). The obtained filter cake was dried overnight at r.t., vacuum <5 mbar.

Salt formation with oxalic acid was tried in acetonitrile. However, no precipitation was observed and the obtained solution was evaporated. TBME was added to the solid residue and sticky material formed. Ethanol was added and a suspension could be stirred at r.t. After filtration, the material was dried overnight under vacuum and then characterized.

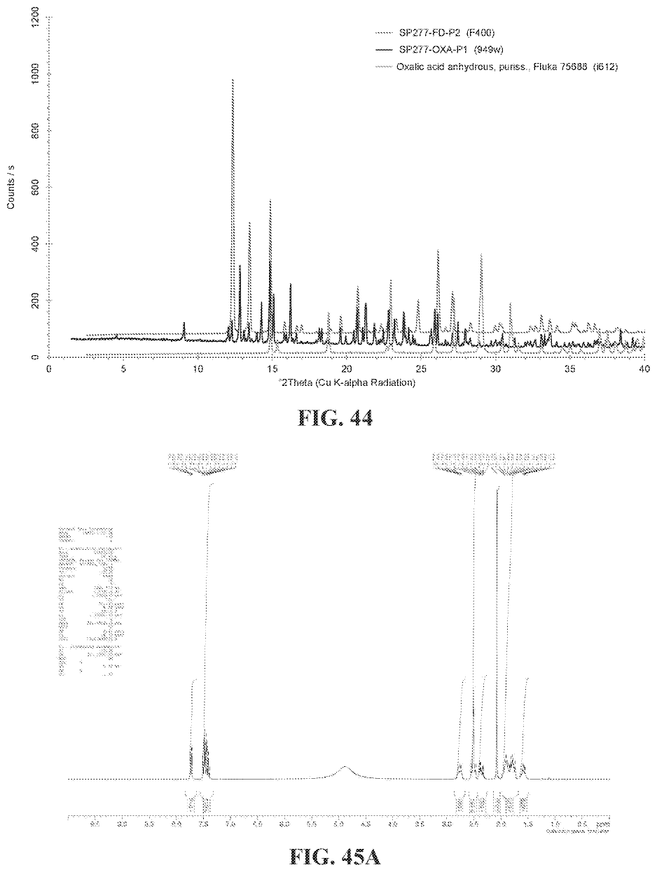

PXRD analysis revealed a new and crystalline pattern (FIG. 43). FIG. 44 shows comparison of the PXRD patterns of this experiment, the free base starting material and oxalic acid.

The ¹H-NMR spectrum of this sample is consistent with the R-ketamine structure and as for the previous salts, changes compared to the spectrum of the free base suggest a new structural environment and therefore salt formation can be considered (FIG. 45A). Further aspects of the chemical identity were verified by elemental composition analysis using CHN, O and Cl content determinations. The obtained results are summarized in Table 18. These results confirm the obtaining of a free base to oxalic acid 2:1 ratio.

### Scale-Up Experiment

R-ketamine free base (1.5192 g, 6.4 mmol) was dissolved in 15 mL of ethyl acetate at 40° C. with sonication treatment. 288 mg of oxalic acid (3.2 mmol, 0.5 eq) was dissolved in 3 mL of ethyl acetate at 40° C. Then the oxalic solution was slowly added to the free base solution under stirring at r.t. After the addition precipitation was observed but transformed rapidly into sticky material. After overnight stirring at r.t., a white suspension was obtained and some sticky material was still observed on the reactor side. After another day of stirring at r.t., the suspension was filtered over fritted glass filter (porosity 4). The reactor was flushed with 3 mL of the mother liquor. The obtained filter cake was dried on the filter for 1 hour with applied vacuum. 955 mg of white powder was recovered (yield of 63%). PXRD pattern corresponds to the previously identified form. NMR spectrum is consistent with R-ketamine structure. Trace of ethyl acetate detected (0.02 eq).

TABLE 18
Result from CHNO and Cl content analysis for R-ketamine
Oxalate Salt sample compared with the theoretical composition
of a solvent-free one to one oxalate salt with a molecular
mass of 327.76 g/mol and the formula C₁₅H₁₈NO₅Cl
and with a hemioxalate salt; i.e., free base to oxalic
acid 2:1, with a molecular mass of 282.75 g/mol and the
formula C₁₄H₁₇NO₃Cl.

| | | Expected | Expected |
|---|---|---|---|
| | Experimental | content for | content for |
| | R-ketamine | an oxalate | an oxalate |
| Element | oxalate salt | salt 1:1 | salt 2:1 |
| C | 58.2% | 55.0% | 59.5% |
| H | 6.0% | 5.5% | 6.1% |
| N | 4.6% | 4.3% | 5.0% |
| O | 19.2% | 24.4% | 17.0% |
| Cl | 12% | 10.8% | 12.5% |
| Solvent Content | — | 0% | 0% |

TG-FTIR shows only a trace of residual water (0.2%, FIG. 45B). DSC thermogram for sample of the R-ketamine oxalate salt is depicted in FIG. 45C and revealed a broad endothermic signal with a maximum at 115° C. which probably corresponds to the melting peak of the oxalate salt.

An FT-Raman spectrum was recorded for sample of the R-ketamine oxalate salt. FIG. 45D shows the full range of the spectrum and FIG. 45E shows the zoomed-in fingerprint region (from 200 cm-1 to 2000 cm-1).

DVS measurement revealed a very hygroscopic oxalate salt with more than 80% water uptake upon storage at 95% relative humidity. The result of the DVS test is presented in FIG. 45F and FIG. 45G. PXRD investigation was conducted on the sample after DVS and dramatically loss of crystallinity was observed FIG. 45H.

### Example 10. R-Ketamine Citrate Salt

R-ketamine free base (191.1 mg, 0.804 mmol) was dissolved in 4 mL of 2-propanol under sonication treatment at r.t. and 77.2 mg of citric acid (0.5 eq, 0.402 mmol) was dissolved in 2.5 mL of 2-propanol under sonication treatment at r.t. The citric acid solution was slowly added to the free base solution under stirring. After the addition, no precipitation was observed and the solution was further stirred at r.t., open vial. After one day, no change was observed, and further evaporation was conducted under nitrogen flow. A gel with glassy residue was obtained and 500 μL of TBME was added. Precipitation was observed. After two days of stirring at r.t., the suspension was filtered using centrifugal unit filter (PTFE, 0.22 μm, 5 min, 5000 rpm, r.t.). The obtained filter cake was dried overnight at r.t., vacuum <5 mbar.

Salt formation was tried with R-ketamine and citric acid in 2-propanol. However, no precipitation was observed, and the obtained solution was evaporated. TBME was added to the solid residue and suspension formed. After filtration, the material was dried overnight under vacuum and then characterized.

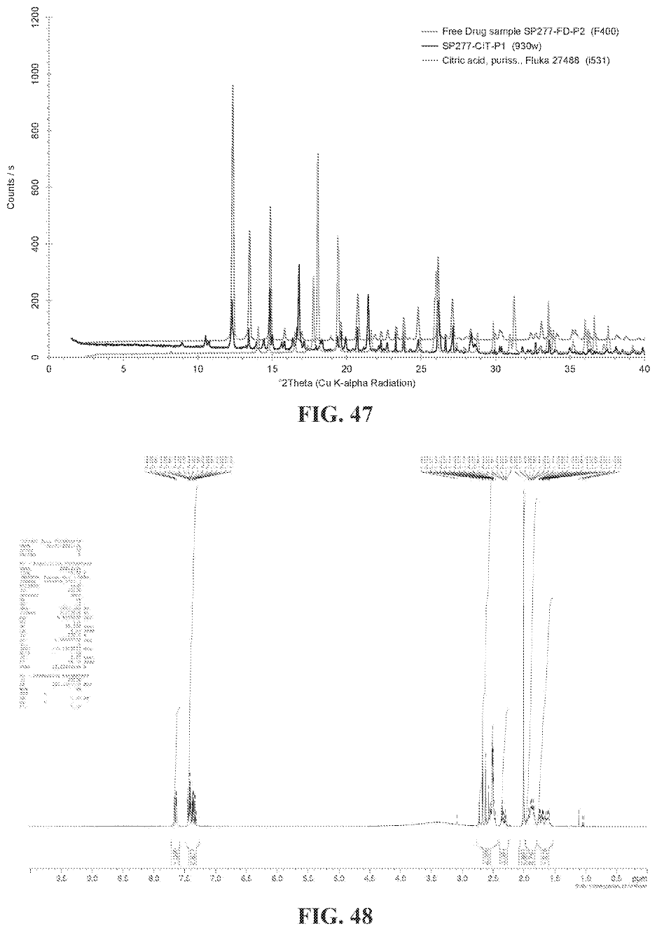

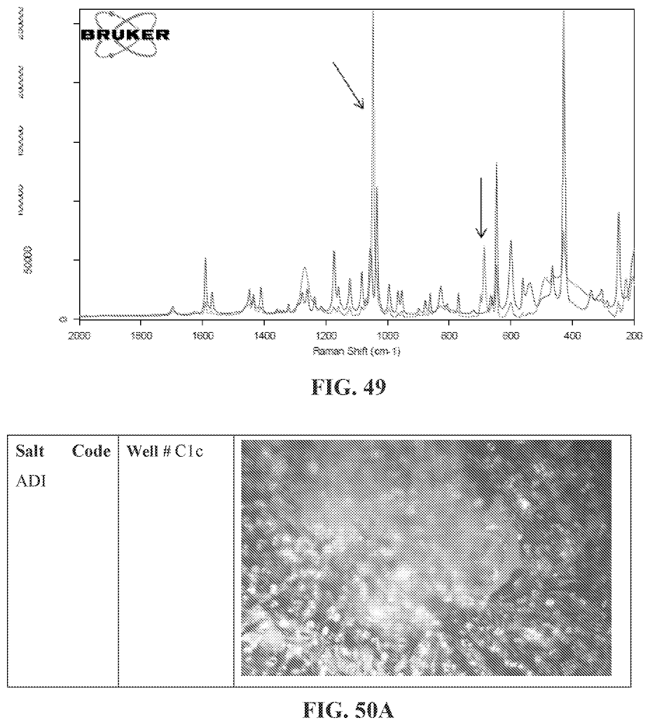

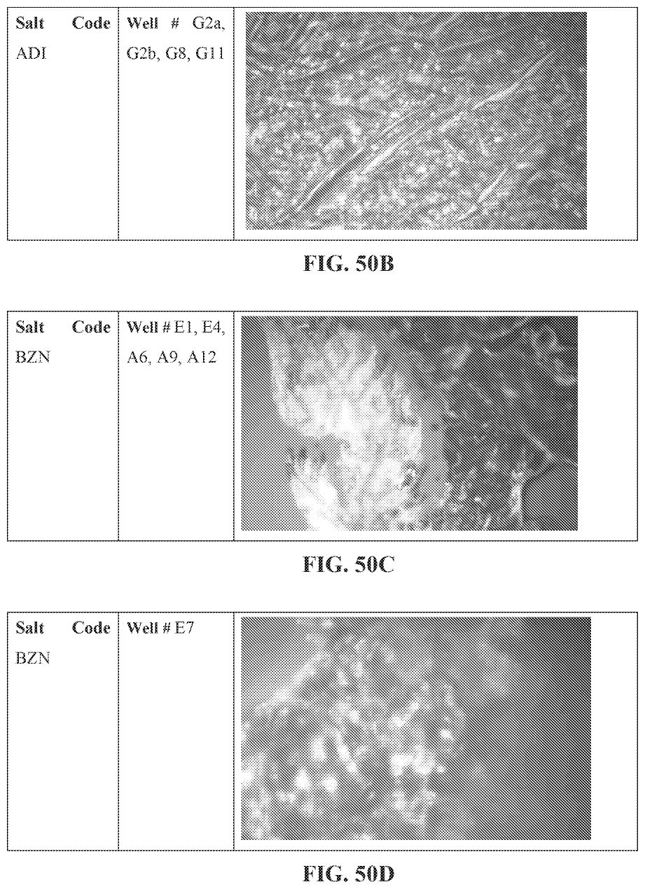

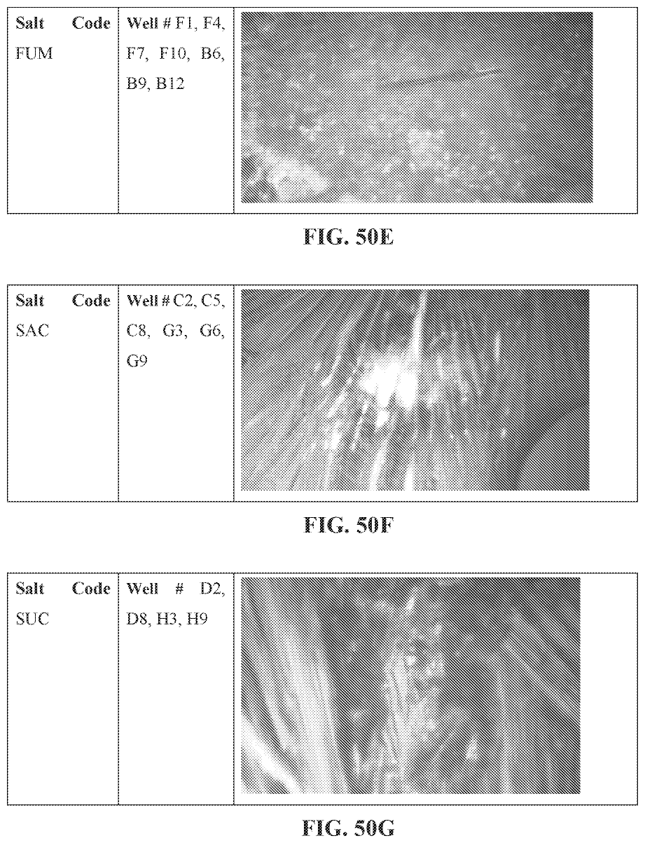

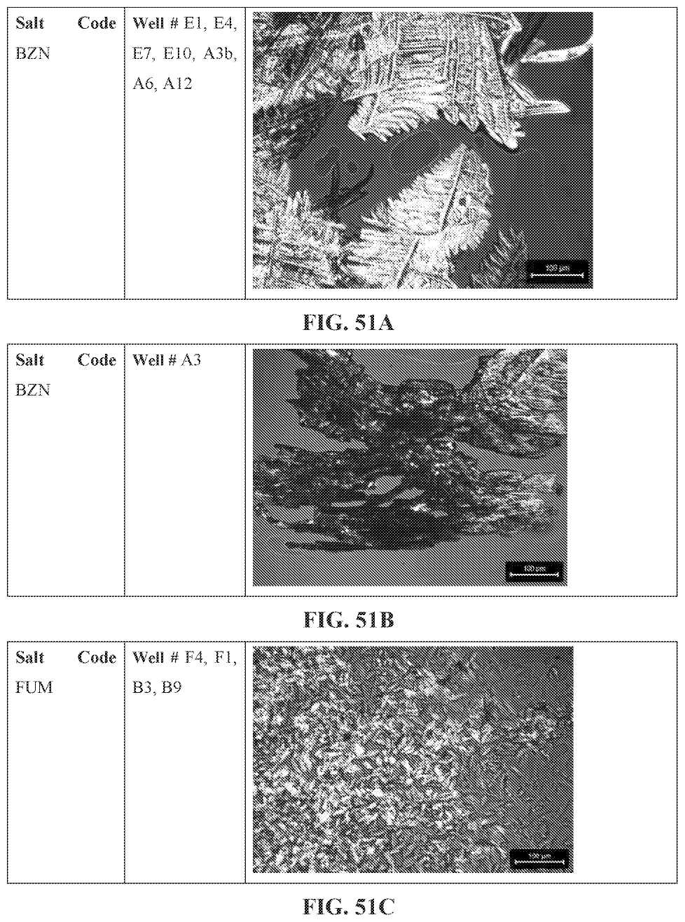

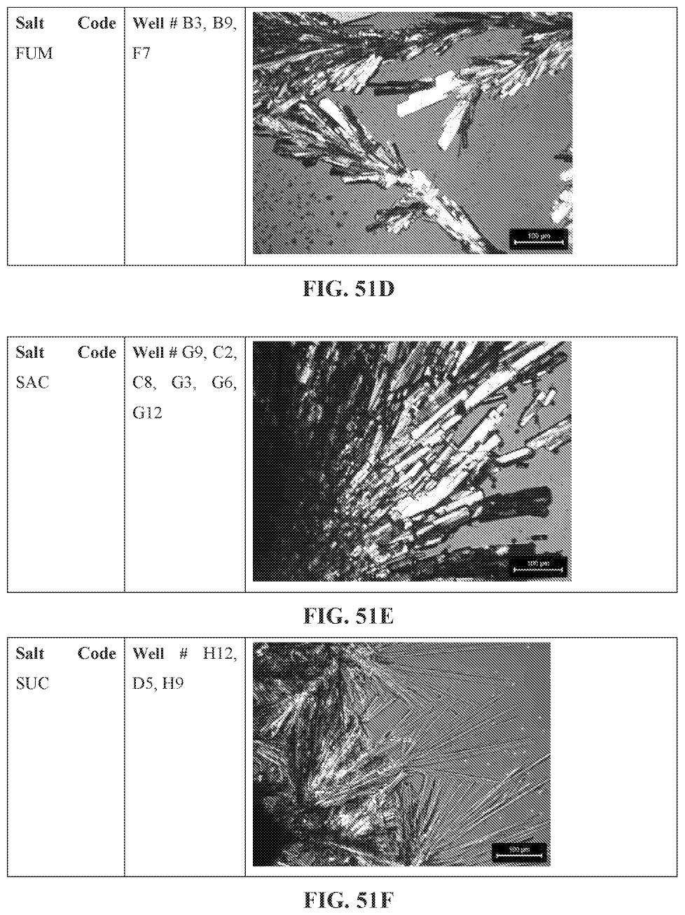

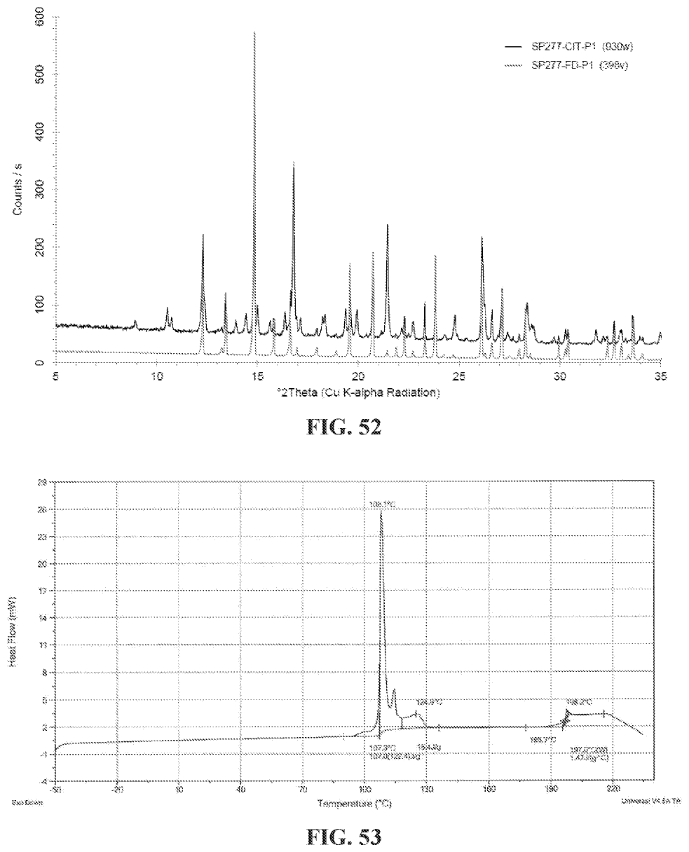

PXRD analysis revealed crystalline material (FIG. 46); the obtained PXRD pattern contains some reflection of the R-ketamine free base with additional reflections which are not attributed to citric acid (FIG. 47). Therefore, this sample is not a pure phase but believed to contain a citrate salt, tentatively a monocitrate salt. Thus an excess amount of free base is conceivable. The overlay with the R-ketamine free base is shown in FIG. 52.

¹H-NMR was conducted on the sample and the spectrum is shown in FIG. 48. Residual 2-propanol (1.04 ppm) and residual TBME (1.11 ppm and 3.08 ppm) are observed. The interpretation is difficult in the range 2 to 3 ppm because of peak superposition. Otherwise the signals are consistent with the R-ketamine structure. The quadruplet at 2.6 ppm cannot be attributed to the R-ketamine and is very unlikely attributable to citric acid.

### Example 11. pH and Solubility Determinations

Precise solubility determinations were conducted for the salts of R-ketamine with saccharin, fumaric acid and oxalic acid in water at 25° C. 50 mg of R-ketamine saccharin salt was suspended in 0.5 mL of water, and the suspension was equilibrated (700 rpm) at 25° C. for one day. 10 to 15 mg of R-ketamine fumarate salt and R-ketamine oxalate salt were also equilibrated in 0.5 mL of water at 25° C. for one day. After 5 minutes of equilibration, a solution was obtained for the fumarate sample and solutions with some small particles were observed for the fumarate and oxalate salts after one day of equilibration3. Filtration was then conducted and the recovered saturated solutions were submitted for HPLC; furthermore the pH was measured at 24° C. (Table 19).

While the R-ketamine hydrochloride salt has a relatively high solubility in water (63<S<125 mg/mL), the solubility of the saccharin salt is lower with 6.6 mg/mL. The solubilities of the R-ketamine fumarate and the R-ketamine oxalate salts are higher than the solubility of the R-ketamine saccharin salt.

A resulting pH of 5.7 was obtained for the oxalate after one day stirring; this result is interesting because this pH is almost neutral.

TABLE 19
HPLC precise solubility determination for the R-ketamine salts.
The pH of the saturated solution was determined at 24° C.

| | Solubility | Initial | pH after |
|---|---|---|---|
| | [mg/mL] | pH | 24 hours |
| R-ketamine | 6.64 | 4.2 (after 1 h) | 4.0 |
| saccharin salt | | | |
| R-ketamine | 17.7* | 4.1 (after 3 h) | 4.1 |
| fumarate salt Form | | | |
| A | | | |
| R-ketamine oxalate | 19.2* | 5.6 (after 3 h) | 5.7 |
| salt | | | |

*Because solutions with some particles were obtained after one day of equilibration, the obtained solubility values may be underestimated.

Furthermore, approximate solubility determinations for the R-ketamine free base, the R-ketamine hydrochloride salt and the R-ketamine saccharin salt were conducted in 20% (w/w) aqueous (2-Hydroxypropyl)-Beta-cyclodextrin (cyclodextrin) solution and also in 20% (w/w) cyclodextrin in buffer pH 7. These values were determined by adding small aliquots of solvent or solvent mixture to ˜10 mg of solid and shaking/sonicating for a short period of time at ambient conditions. The solubilities were estimated by visual observations. These values are only approximations. Table 20 summarized the obtained solubility values, and the pH of the resulting solutions.

TABLE 20
Solubility values of the R-ketamine free base, the R-ketamine
HCl salt and the R-ketamine saccharin salt in 20% (w/w) aqueous
cyclodextrin solution and in 20% (w/w) cyclodextrin in buffer
pH 7. The pH of the obtained solutions was measured at 24° C.

| | |
|---|---|
| Approximate Solubility [mg/mL] | pH |

20% (w/w) aqueous cyclodextrin

| | | |
|---|---|---|
| Free base | 1.5~1.7 | 8.5 |
| HCl salt | 199~249 | 4.1 |
| Saccharin salt | 28~37 | 5 |

20% (w/w) cyclodextrin in buffer pH 7 solution

| | | |
|---|---|---|
| Free base | 2~2.5 | 7.1 |
| HCl salt | 191~239 | 5.4 |
| Saccharin salt | 26~35 | 6.3 |

### Example 12. R-Ketamine D-Tartrate Salt Form B

R-ketamine free base (293.4 mg, 1.23 mmol) was dissolved in 2.0 mL of acetone at r.t. under sonication treatment. 92.6 mg of D-tartaric acid (0.62 mmol, 0.5 eq) was dissolved in 3.0 mL of ethanol at r.t. under sonication treatment. Then the tartaric solution was slowly added to the free base solution under stirring. Precipitation was immediately observed with some sticky material. After four days stirring at r.t., a white suspension was obtained and filtration was conducted using centrifugal unit filter (PTFE, 0.22 μm, 5 min, 5000 rpm, r.t.). The obtained filter cake was dried overnight at r.t., vacuum <5 mbar. 213 mg of substance was recovered. A sample with a new PXRD pattern was obtained (FIG. 56). The pattern is different from D-tartaric acid as shown in FIG. 57. This pattern is different from the previous R-ketamine D-tartrate salt Form A and from the free base; an overlay of the three patterns is depicted in FIG. 74. NMR spectrum is consistent with R-ketamine structure.

¹H-NMR on sample R-ketamine D-tartrate Form B revealed one equivalent of tartaric acid; therefore a 1:1 salt was obtained in FIG. 58. Residual (0.01 eq) acetone is also visible.

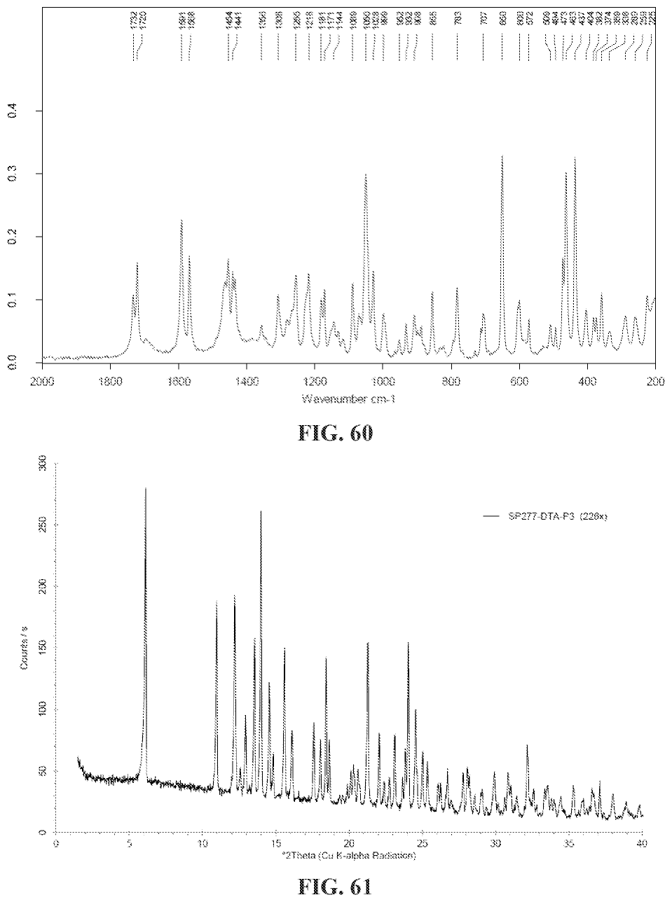

An FT-Raman spectrum was recorded R-ketamine D-tartrate Form B. FIG. 59 shows the full range of the spectrum and FIG. 60 shows the zoomed-in fingerprint region (from 200 cm-1 to 2000 cm-1).

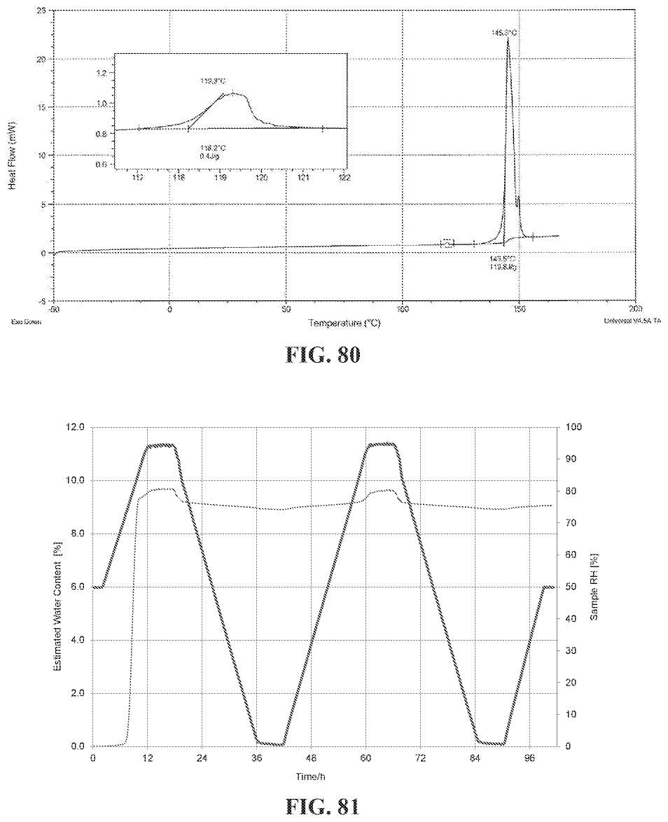

Reanalysis of the material after 12 months of storage in a closed vial at ambient conditions gave a PXRD pattern corresponding to R-ketamine D-tartrate salt Form B. TG-FTIR analysis shows that the crystalline form is essentially free of residual solvent, however, thermal decomposition begins at about 150° C. (FIG. 79). DSC measurement revealed the main melting peak at 145° C. From the DSC result it is not possible to determine as to whether R-ketamine D-tartrate salt Forms B and C are monotropically or enantiotropically related. Based on the small additional peak at 150° C., it seems that R-ketamine D-tartrate salt Form B has partially converted to R-ketamine D-tartrate salt Form C during the DSC test. Due to the suspected phase conversion a meaningful value for the enthalpy of fusion cannot be evaluated. The very small signal at 119° C. likely corresponds to a trace of free base present in the sample (see FIG. 80).

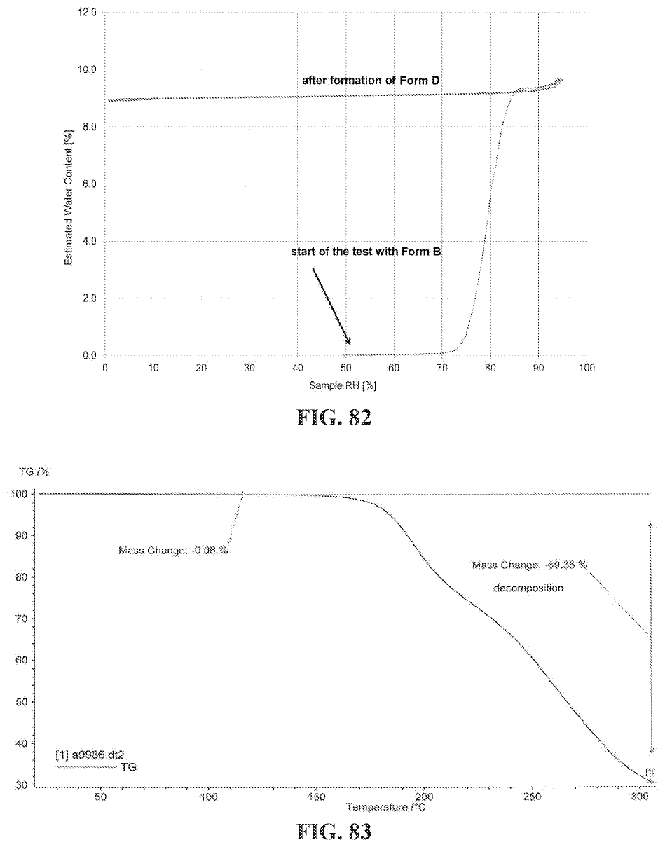

The material was also examined by DVS. The result from the DVS test is presented in FIG. 81 and FIG. 82. Above 70% relative humidity, R-ketamine D-tartrate salt Form B begins to absorb substantial amounts of water that goes up to a water content of about 8.5%. In course of the measurement, this water content does not change much anymore. The water sorption is irreversible under the test conditions and corresponds to the water content of R-ketamine D-tartrate salt Form D. The solid recovered after the DVS test was investigated using FT-Raman, which corresponds to R-ketamine D-tartrate salt Form D.

### Example 13. R-Ketamine D-Tartrate Salt Form C (MeOH)

R-ketamine free base (249.2 mg, 1.05 mmol) was dissolved in 2.0 mL of methanol at r.t. under sonication treatment. 78.7 mg of D-tartaric acid (0.52 mmol, 0.5 eq) was dissolved in 1.0 mL of methanol at r.t. under sonication treatment. Then the tartaric solution was slowly added to the free base solution under stirring. No precipitation was observed after the addition. A solution was still observed after two days of equilibration and the solvent was evaporated with nitrogen flow. After 4 hours, a wet residue was obtained and 1.0 mL of ethyl acetate was added. A solution was obtained after shaking. After overnight stirring, a suspension formed and was then filtered using a centrifugal unit filter (PTFE, 0.22 μm, 5 min, 5000 rpm, r.t.). The obtained filter cake was dried over 2 days at r.t., vacuum <5 mbar. 151.3 mg of substance was obtained. A new PXRD pattern was obtained as the R-ketamine D-tartrate salt Form C, with one additional peak at 14.8° that corresponds to free base. NMR spectrum is consistent with R-ketamine structure.

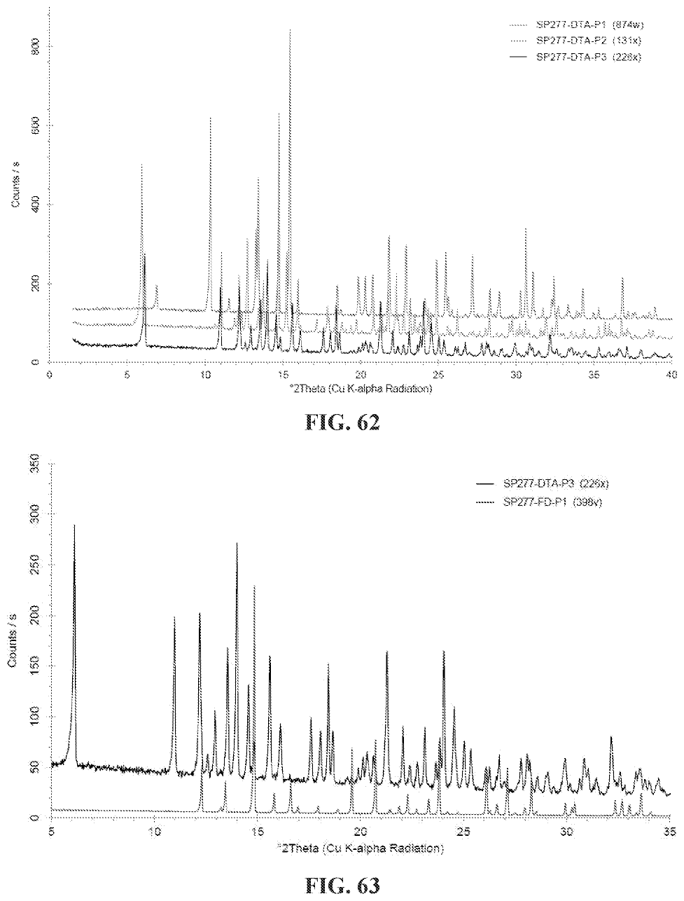

The PXRD pattern for the R-ketamine D-tartrate salt Form C is depicted in FIG. 61. FIG. 62 shows an overlay of the PXRD patterns of three tartrate salts and FIG. 63 shows an overlay of the R-ketamine D-tartrate salt Form C with the R-ketamine free base.

¹H-NMR measurement was conducted on the R-ketamine D-tartrate salt Form C sample and is presented in FIG. 64. A broad signal is detected under the tartaric signal at 4.27 ppm and therefore the integration is difficult. Roughly estimated, the sample contains 1 to 1.5 equivalent of tartaric acid. This is not necessarily contradictive to the X-ray result because the stoichiometry of the new salt could be in favor of tartaric acid.

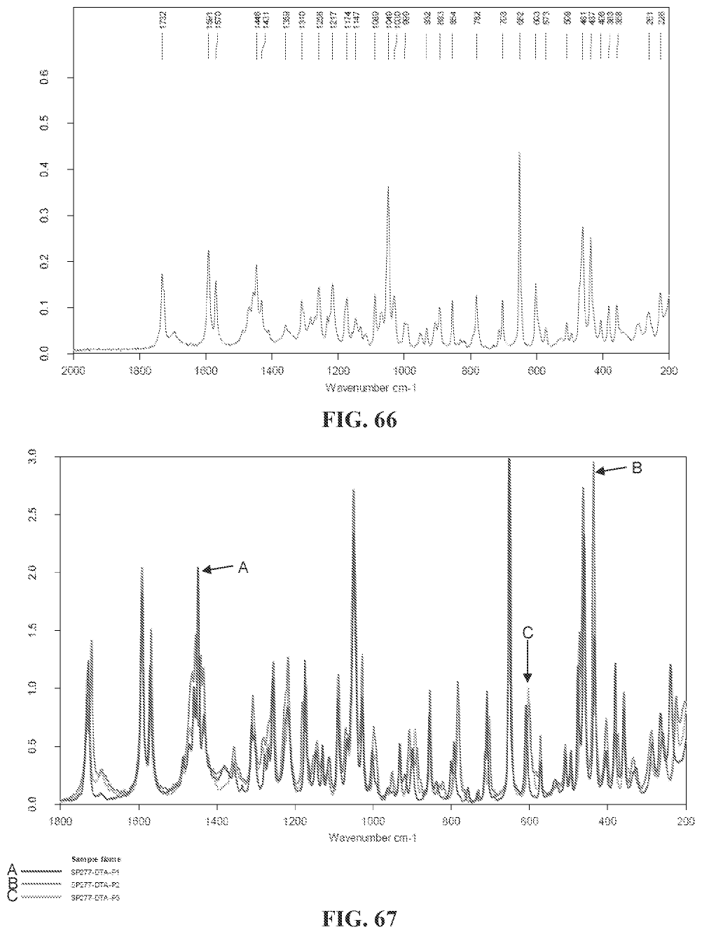

The Raman spectrum for the R-ketamine D-tartrate salt Form C is presented in FIG. 65 and FIG. 66. An overlay of the Raman spectra of all three D-tartrate samples as shown in FIG. 67 and confirms that all solid forms are different in nature.

Reanalysis of the material after 12 months of storage in a closed vial at ambient conditions gave a PXRD pattern corresponding to R-ketamine D-tartrate salt Form C. TG-FTIR analysis showed that R-ketamine D-tartrate salt Form C is essentially free of residual solvent; however, thermal decomposition begins at about 160° C. (FIG. 83)

### Example 14. R-Ketamine D-Tartrate Salt Form C (Ethanol) and Conversion to Form D

At 60° C., 481 mg of R-ketamine free base (˜2.0 mmol) were dissolved in 5.0 mL of ethanol. 6.0 mL of a stock solution of D-tartaric acid in ethanol (0.33 M) was added. The solution remained clear and was placed into a temperature controller to cool to 4° C. After about four hours the solution had changed to a thick suspension. Because it seemed that the solid was possibly amorphous, 2.5 mL of the suspension was stirred at 40° C. overnight and then filtered using a centrifugal unit filter (PTFE, 0.22 μm, 5000 rpm, 2 min, r.t.). The wet cake was submitted for PXRD. PXRD pattern corresponds to R-ketamine D-tartrate salt Form C.

200 μL of water was added to 5.0 mL of the suspension of R-ketamine D-tartrate salt Form C (water activity ˜0.3). The obtained suspension was stirred for one day and then filtered over fritted glass (porosity 4). After a few minutes drying on the filter, the solid was filled into a vial and submitted for PXRD. PXRD pattern corresponds to R-ketamine D-tartrate salt Form D.

### Example 15. R-Ketamine D-Tartrate Salt Form C (Acetone) and Conversion to Form D

At 40° C., 383 mg of R-ketamine free base (˜1.6 mmol) were dissolved in 3.0 mL of acetone. 8.0 mL of a stock solution of D-tartaric acid in acetone (0.20 M) was added. A turbid solution was obtained at 40° C., then a suspension at 35° C. The suspension was removed from temperature controller, but after a few hours of stirring at r.t., a quick test by light microscopy indicated that the material might be poorly crystalline. The suspension was then stirred at 40° C. overnight, and then 2.5 mL were filtered using a centrifugal unit filter (PTFE, 0.22 μm, 5000 rpm, 5 min, r.t.). The wet cake was submitted for PXRD. PXRD pattern corresponds to R-ketamine D-tartrate salt Form C. As shown in Example 12, using a 1:0.5 ratio of R-ketamine free base to D-tartaric acid gives R-ketamine D-tartrate salt Form B. Here, using a 1:1 ratio gives R-ketamine D-tartrate salt Form C.

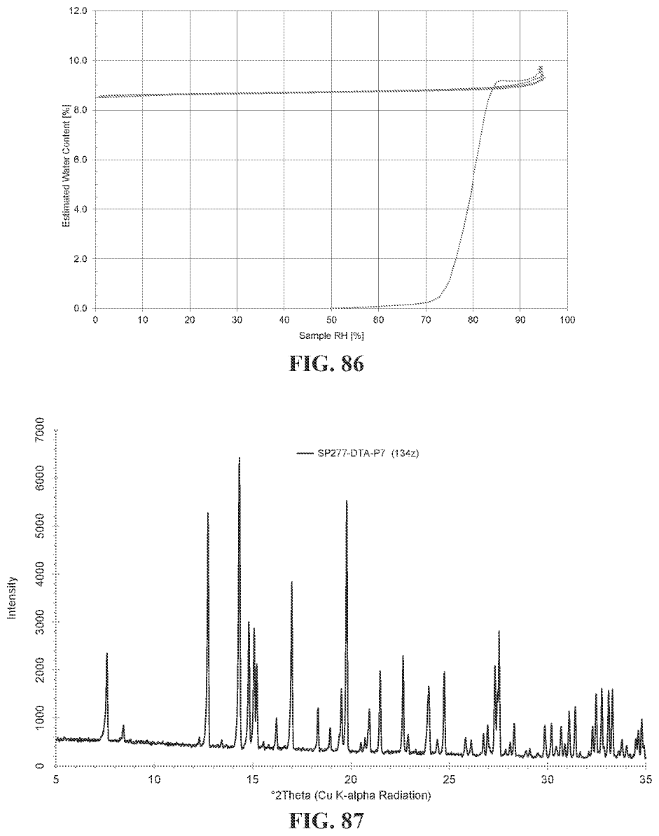

DVS measurement revealed that above 70% relative humidity, R-ketamine D-tartrate salt Form C begins to absorb substantial amounts of water that goes up to a water content of about 8.3% (FIGS. 85, 86). In course of the measurement, this water content does not change much anymore. The water sorption is irreversible under the test conditions and corresponds to the water content of R-ketamine D-tartrate salt Form D. Solid recovered after DVS test was investigated by FT-Raman. FT-Raman spectra corresponds to R-ketamine D-tartrate salt Form D.

Water (500 μL) was added to 5.0 mL of the R-ketamine D-tartrate salt Form C suspension (a_{w}˜0.75). The obtained suspension was stirred for one day at r.t. and filtered over a fritted glass filter (porosity 4). After a few minutes of drying on the filter, the solid was filled into a vial and submitted for PXRD. PXRD pattern corresponds to R-ketamine D-tartrate salt Form D.

### Example 16. R-Ketamine D-Tartrate Salt Form C (IPA) and Conversion to Form D

R-ketamine free base (340 mg) and D-tartaric acid (212 mg) were dissolved in 4.0 mL of IPA by heating to about 70° C. The solution was allowed to cool to r.t. by removing the vial from temperature controller; a suspension was obtained. Because a quick test by light microscopy suggested that the material was poorly crystalline, the suspension was stirred at 40° C. over-night. 2.5 mL of the suspension was then filtered using a centrifugal unit filter. The wet cake was submitted for PXRD. 2.0 mL of the recovered filtrate (saturated solution in isopropanol at approx. 40° C.) was evaporated and the residue was vacuum dried. 7.2 mg of solid residue was obtained. PXRD pattern corresponds to R-ketamine D-tartrate salt Form C.

To the remaining suspension, 2.0 mL of IPA was added. The major part of the suspension (5.0 mL) was transferred into a new vial. Then about 40 μL of water was added (0.8%, aw˜0.1) and the suspension was stirred at r.t. for three days. After filtration over a fritted glass filter, the solid product was let dry for a few minutes, and then submitted for PXRD. PXRD pattern shows a mixture of R-ketamine D-tartrate salt Form C and Form D.

### Example 17. R-Ketamine Fumarate Salt Form B

R-ketamine free base (263.9 mg, 1.11 mmol) was dissolved in 1 mL of THE at r.t. with sonication treatment. 64.4 mg of fumaric acid (0.56 mmol, 0.5 eq) was dissolved in 2 mL of THE at r.t. with sonication treatment. Then the fumaric solution was slowly added to the free base solution under stirring. After the addition a clear solution was obtained but transformed rapidly into a suspension. After two days of stirring, light microscopy revealed crystalline material and the suspension was filtered using centrifugal unit filter (PTFE, 0.22 μm, 5 min, 5000 rpm, r.t.). The obtained filter cake was dried overnight at r.t., vacuum <5 mbar. 129.5 mg of substance was recovered. New PXRD pattern (Form B) which may possibly contain some reflections of Form A. NMR spectrum consistent with structure. 0.5 equivalent of fumaric acid detected. 0.2 eq of THE detected.

Fumaric acid was selected for scale up experiments that were directed towards production of salts with a free base to acid ratio of 2:1. The first experiment led to a crystalline hemifumarate salt Form A which was characterized by PXRD, 1H-NMR, FT-Raman, TG-FTIR, DSC, DVS, and aqueous solubility at room temperature.

Surprisingly when a second experiment was carried out in a different solvent, the obtained material appeared to contain another crystalline form—Form B. Therefore it appears that the hemifumarate exists in at least two different crystalline forms.

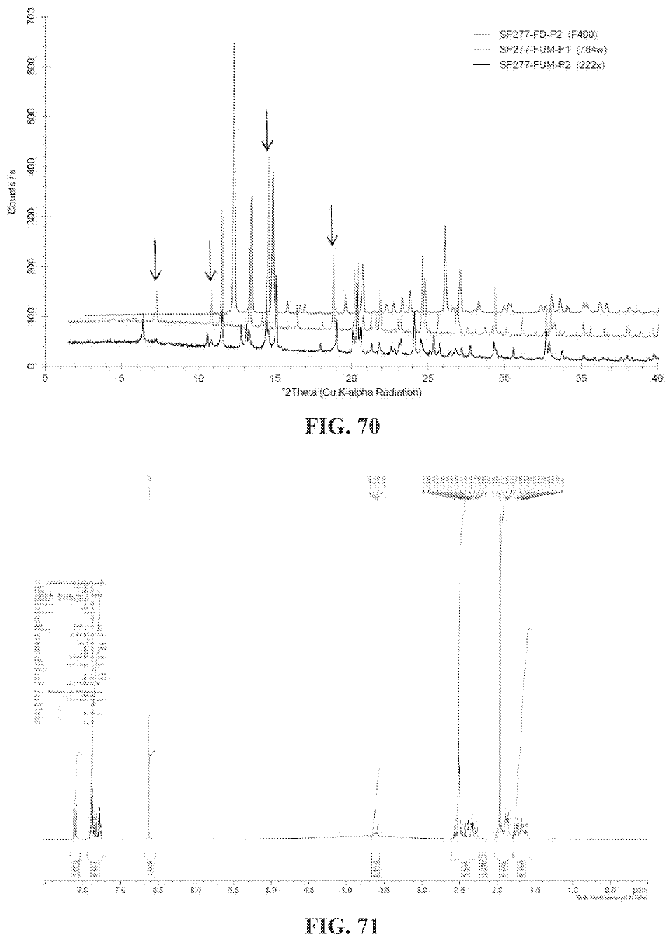

A new PXRD pattern that showed crystalline material was obtained from this experiment and is depicted in FIG. 68. The R-ketamine fumarate salt Form B is distinct from fumaric acid as shown in FIG. 69. FIG. 70 shows a comparison of this new PXRD pattern the R-ketamine fumarate salt Form B with the free base and the R-ketamine fumarate salt Form A. It seems that the pattern of the R-ketamine fumarate salt Form B contains a small amount of the form of the R-ketamine fumarate salt Form A.

¹H-NMR revealed 0.5 equivalent of fumaric acid and confirmed the free base to acid 2:1 ratio. The ¹H-NMR is depicted in FIG. 71; residual THF (0.2 eq) is detected at 3.6 ppm. Therefore it appears conceivable that the hemifumarate exists in at least two different crystalline forms.

An FT-Raman spectrum was recorded for sample of the R-ketamine fumarate salt Form B. FIG. 72 shows the full range of the spectrum and FIG. 73 shows the zoomed-in fingerprint region (from 200 cm-1 to 2000 cm-1).

After 11 months of storage in a closed vial at ambient condition, the material was retested by PXRD, which gave a mixture of the R-ketamine fumarate salt Form A and B (about 1:1).

### Example 18. R-Ketamine L-Tartrate Salt Form a (Ethanol)

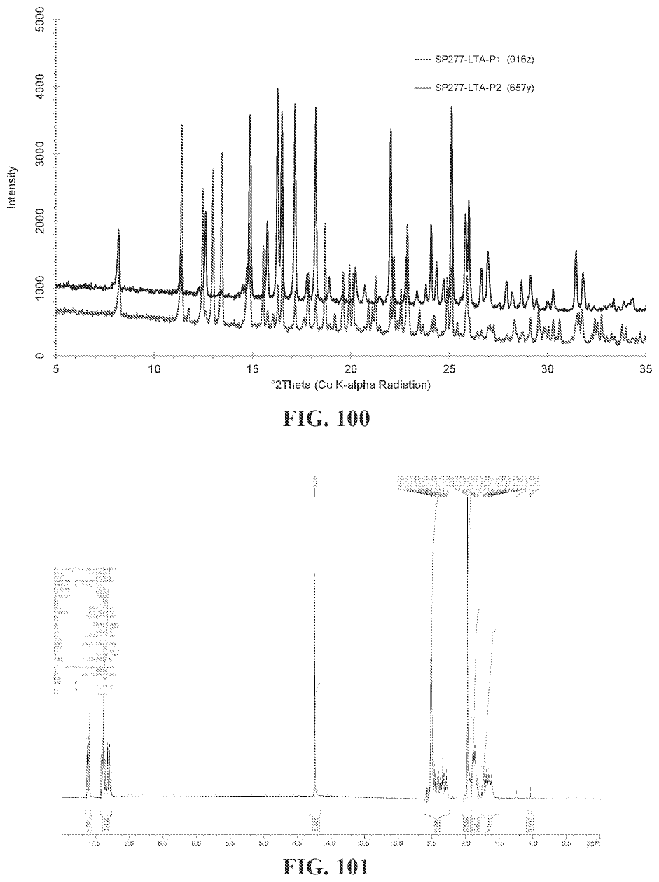

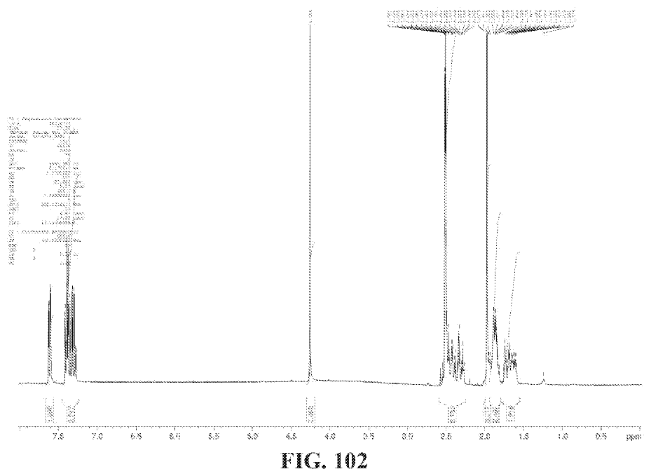

R-ketamine free base (202.6 mg, 0.85 mmol) was dissolved in 3 mL of ethanol at 40° C. with sonication treatment. 64.1 mg of L-tartaric acid (0.43 mmol, 0.5 eq) was dissolved in 1.5 mL of ethanol at 40° C. Then the tartaric solution was slowly added to the free base solution under stirring at r.t. After the addition a clear solution was observed. After overnight stirring at r.t., no precipitation was observed and the cap of the reactor was pierced with two needles to allow slow solvent evaporation. After three days of stirring at r.t, a very fine suspension was obtained. The reactor was opened and the suspension was further stirred at r.t. All the solvent was evaporated and a dried crystalline residue was obtained. 2 mL of isopropanol was added to the residue and a suspension was obtained. After one week of stirring, the suspension was filtered using a centrifugal unit filter (PTFE, 0.22 μm, 5000 rpm, 5 min, r.t.). The recovered filter cake was vacuum dried for 1 hour and then analyzed using PXRD and ¹H-NMR (FIGS. 98, 101).

### Example 19. R-Ketamine L-Tartrate Salt Form B (THF)

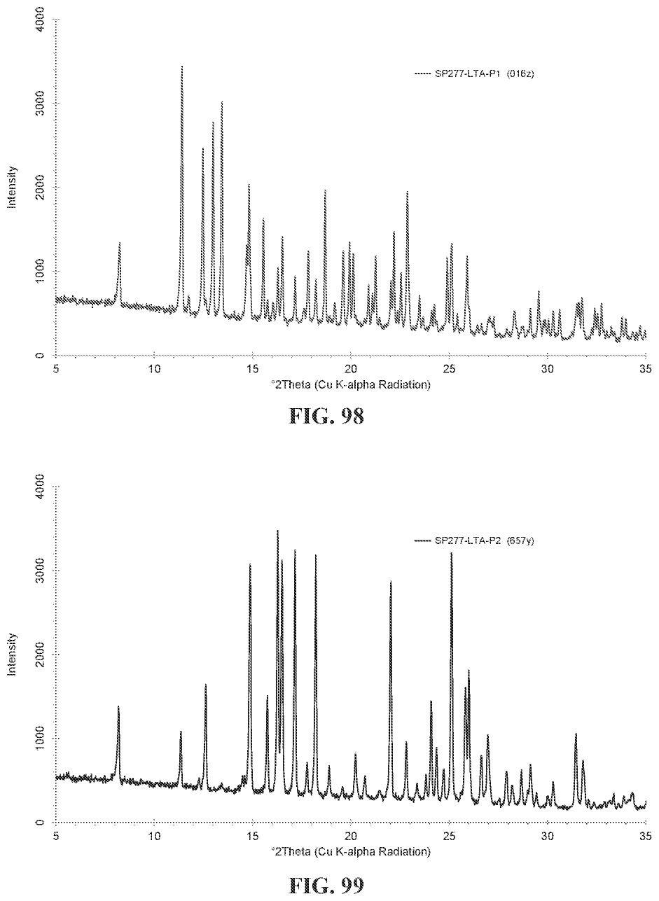

R-ketamine free base (195.8 mg, 0.82 mmol) was dissolved in 2 mL of THF at 40° C. with sonication treatment. 62.1 mg of L-tartaric acid (0.41 mmol, 0.5 eq) was dissolved in 1.5 mL of THF at 40° C. Then the tartaric solution was slowly added to the free base solution under stirring at r.t. After the addition a clear solution was observed but transformed rapidly into a suspension. After overnight stirring at r.t., the white suspension was filtered using a centrifugal unit filter (PTFE, 0.22 μm, 5000 rpm, 5 min, r.t.). The recovered filter cake was dried in air over the weekend. The NMR spectrum is consistent with R-ketamine structure (FIG. 102). 1 eq of L-tartaric acid was detected. Only traces of THF were visible. A new and crystalline PXRD was obtained (FIG. 99).

### Example 20. R-ketamine L-tartrate Salt (Acetone)

R-ketamine free base (199.4 mg, 0.84 mmol) was dissolved in 2 mL of acetone at 40° C. with sonication treatment. 62.8 mg of L-tartaric acid (0.42 mmol, 0.5 eq) was dissolved in 3 mL of acetone at 40° C. Then the tartaric solution was slowly added to the free base solution under stirring at r.t. After the addition a clear solution was observed but transformed rapidly into a suspension. After overnight stirring at r.t., the white suspension was filtered using a centrifugal unit filter (PTFE, 0.22 μm, 5000 rpm, 5 min, r.t.). The recovered filter cake was dried in air over the weekend. PXRD pattern corresponds to R-ketamine L-tartrate salt form A with small additional less intense reflections (13°, 16°, 18.7°, 21.2°, 21.3°, 22.5°, and 23° 2-theta). The NMR spectrum is consistent with R-ketamine structure. 1 eq. of L-tartaric acid was detected. Only traces of acetone were visible (0.012 eq).

### Example 21. R-Ketamine L-Tartrate Salt (IPA)

400 mg R-ketamine free base and 250 mg of L-tartaric acid were dissolved in 5.0 mL of IPA by heating to about 78° C. A clear solution was obtained that was allowed to cool by setting the temperature controller to 40° C. and stirred overnight. A white suspension was obtained which was then filtered using a centrifugal unit filter.

2.0 mL of the filtrate (saturated solution in isopropanol at approx. 40° C.) were evaporated and the residue was vacuum dried. 15.4 mg of solid residue was weighed out. PXRD pattern corresponds to R-ketamine L-tartrate salt Form B. Gravimetric solubility: S˜7.7 mg/mL

### Example 22. R-Ketamine D-Tartrate Salt Form D (1-Propanol)

R-ketamine free base (203.8 mg, 0.86 mmol) was dissolved in 4 mL of 1-propanol at 40° C. with sonication treatment. 64.2 mg of D-tartaric acid (0.43 mmol, 0.5 eq) was dissolved in 2 mL of 1-propanol at 40° C. Then the tartaric solution was slowly added to the free base solution under stirring at r.t. After the addition a clear solution was observed. After overnight stirring at r.t., no precipitation was observed and the cap of the reactor was pierced with two needles to allow slow solvent evaporation. After three days of stirring at r.t, a fine suspension was obtained. The reactor was opened and the suspension was further stirred at r.t. for 1 day. Then filtration was conducted using a centrifugal unit filter (PTFE, 0.22 μm, 5000 rpm, 5 min, r.t.). The recovered filter cake was dried in air overnight. PXRD pattern is a new R-ketamine Form D. NMR spectrum is consistent with R-ketamine structure (FIG. 95). 1.3 eq of D-tartaric acid was detected. (large signal from 3 to 6 ppm). Comparison of the PXRD pattern of R-ketamine D-tartrate salt Form D with a previously published PXRD pattern of S-ketamine L-tartrate dihydrate (calculated from SC-XRD data that was measured at −130° C. Taken from E. Ratti-Moberg, P. Groth, A. J. Aasen, Acta Chem. Scand., 1991, 45, 108) indicates that R-ketamine D-tartrate salt form D is isostructural to the known form of S-ketamine L-tartrate dihydrate (FIG. 97).

Reanalysis of the material after 6 months of storage in a closed vial at ambient conditions gave a PXRD pattern corresponding to R-ketamine D-tartrate salt Form D.

An FT-Raman spectrum was recorded for sample of the R-ketamine D-tartrate salt Form D. FIG. 88 shows the full range of the spectrum and FIG. 89 shows the zoomed-in fingerprint region (from 200 cm-1 to 1800 cm-1).

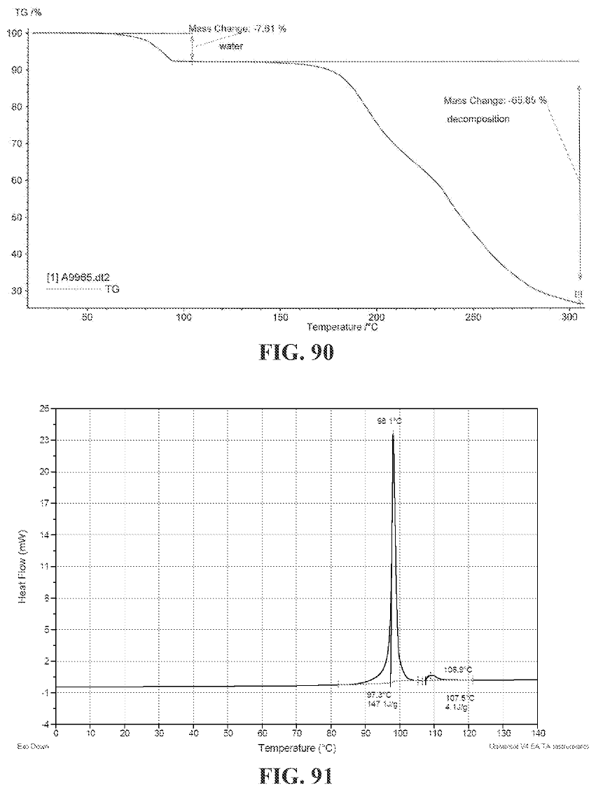

TG-FTIR analysis showed a mass loss of 7.6% which is attributable to water (FIG. 90).

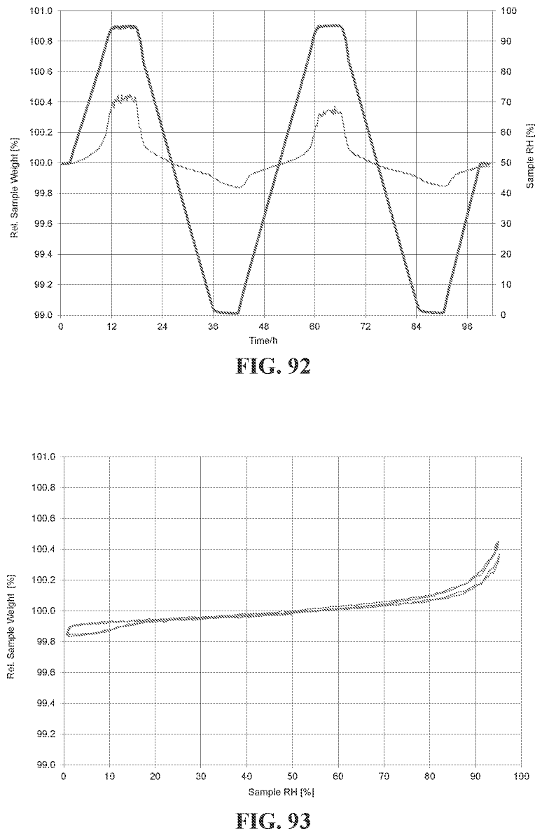

DSC measurement revealed a melting peak at 98° C., with an associated enthalpy of 147 J/g (FIG. 91). DVS measurement shows small change of the water content throughout the measurement (FIGS. 92 and 93). About 0.4% additional water is absorbed at high relative humidity and about 0.1% to 0.2% water is lost during the four hours equilibration time at 0% humidity. Solid recovered after DVS test was investigated by FT-Raman. FT-Raman corresponds to R-ketamine D-tartrate salt Form D.

### Example 23. R-Ketamine D-Tartrate Salt Form D (THF)

R-ketamine free base (200.3 mg, 0.84 mmol) was dissolved in 2 mL of THF at 40° C. with sonication treatment. 63.7 mg of D-tartaric acid (0.42 mmol, 0.5 eq) was dissolved in 1.5 mL of THF at 40° C. Then the tartaric solution was slowly added to the free base solution under stirring at r.t. After the addition a clear solution was observed but transformed rapidly into a suspension. After overnight stirring at r.t., the white suspension was filtered using a centrifugal unit filter (PTFE, 0.22 μm, 5000 rpm, 5 min, r.t.). The recovered filter cake was dried in air over the weekend. PXRD pattern is crystalline and corresponds to a combination of Form C and Form D. NMR spectrum is consistent with R-ketamine structure. 1.2 eq of D-tartaric acid was detected. No residual THF detected.

### Example 24. R-Ketamine D-Tartrate Salt Form D (2-Propanol)

R-ketamine free base (214.5 mg, 0.90 mmol) was dissolved in 5 mL of 2-propanol at 40° C. with sonication treatment. 68 mg of D-tartaric acid (0.45 mmol, 0.5 eq) was dissolved in 2 mL of 2-propanol at 40° C. Then the tartaric solution was slowly added to the free base solution under stirring at r.t. After the addition a clear solution was observed but transformed into a suspension after two hours of stirring. After overnight stirring at r.t., the white suspension was filtered using a centrifugal unit filter (PTFE, 0.22 μm, 5000 rpm, 5 min, r.t.). The recovered filter cake was dried in air over the week-end. PXRD pattern corresponds to a combination of Form C and Form D with orientation effects. NMR spectrum is consistent with R-ketamine structure. 1.3 eq of D-tartaric acid was detected (large signal from 3 to 6 ppm). No residual THF detected. Trace of 2-propanol.

### Example 25. R-Ketamine D-Tartrate Salt Form D (MeCN)

R-ketamine free base (207 mg, 0.87 mmol) was dissolved in 2 mL of acetonitrile at r.t. 65 mg of D-tartaric acid (0.44 mmol, 0.5 eq) was dissolved in 0.5 mL of water at r.t. Then the tartaric solution was slowly added to the free base solution under stirring at r.t. After the addition a clear solution was observed. After overnight stirring at r.t., no precipitation was observed and the cap of the reactor was opened to allow slow solvent evaporation. After three days a wet residue was obtained and was further dried with nitrogen flow. 0.5 mL of acetonitrile was added to the dried residue. A nice suspension was obtained and after one day of stirring at r.t., the suspension was filtered using a centrifugal unit filter (PTFE, 0.22 μm, 5000 rpm, 5 min, r.t.). The recovered filter cake was dried in air overnight. PXRD pattern is a new R-ketamine Form D.

Reanalysis of the material after 4 months of storage in a closed vial at ambient conditions gave a PXRD pattern corresponding to R-ketamine D-tartrate salt Form D. 7.55% water loss from 25 to 105° C.

### Example 26. R-Ketamine Fumarate Scale Up and Repeat Experiments

R-ketamine free base (1.50 g, 6.32 mmol) was dissolved in 20 mL of ethanol at r.t. with sonication treatment. 366.16 mg of fumaric acid (3.14 mmol, 0.5 eq) was dissolved in 10 mL of ethanol at r.t. with sonication treatment. Then the fumaric solution was slowly added to the free base solution under stirring. A suspension started to form very slowly. The mixture was further dried with open vial, in order to allow solvent evaporation. After overnight stirring, the suspension was filtered over fritted glass filter (porosity 4). The recovered wet filter cake was dried under vacuum (<10 mbar, r.t.) overnight. 0.253 g of material was recovered. PXRD pattern corresponds to Form A with two additional reflections (12.9 and 14.2° 2-theta). NMR spectrum is consistent with structure R-ketamine structure. 0.63 eq of fumaric acid detected. Small signal at 2.7 ppm which can not be attributed.

Precipitation was observed in the mother liquor after filtration. The mother liquor was evaporated with N2 flow to ⅓ of the volume; a white suspension was obtained. After overnight stirring, the suspension was filtered over fritted glass filter (porosity 4). The recovered wet filter cake was dried under vacuum (<10 mbar, r.t.) overnight. 0.87 g of material was recovered. PXRD pattern corresponds to Form A.

R-ketamine free base (1.506 g, 6.32 mmol) was dissolved in 6 mL of THE at r.t. with sonication treatment. 366.20 mg of fumaric acid (3.14 mmol, 0.5 eq) was dissolved in 12 mL of THE at r.t. with sonication treatment. Then the fumaric solution was slowly added to the free base solution under stirring. After the addition a clear solution was obtained but transformed rapidly into a suspension. After overnight stirring at r.t., the suspension was filtered over fritted glass filter (porosity 4). The recovered wet filter cake was dried under vacuum (<10 mbar, r.t.) overnight. 0.816 g of material was recovered. PXRD pattern corresponds to Form A with one additional reflection at 14.8° 2-theta. NMR spectrum is consistent with R-ketamine structure. 0.5 eq of fumaric acid detected. 0.08 eq of THE detected.

Precipitation was observed in the mother liquor after filtration and the solid was filtered over fritted glass filter (porosity 4). The recovered wet filter cake was dried under vacuum (<10 mbar, r.t.) overnight. 0.349 g of material was recovered. PXRD pattern corresponds to Form A.

R-ketamine free base (263.77 mg, 1.11 mmol) was dissolved in 1 mL of THF at r.t. A cloudy solution was obtained an additional 1 mL of THE was added. A solution was obtained after sonication treatment. 64.35 mg of fumaric acid (0.56 mmol, 0.5 eq) was dissolved in 2 mL of THE at r.t. with sonication treatment. Then the fumaric solution was slowly added to the free base solution under stirring. After the addition a cloudy solution was obtained. After overnight stirring at r.t., a fine suspension was observed and the vial was opened to allow solvent evaporation. A suspension formed and was further stirred at r.t. overnight. The white suspension was filtered using a centrifugal unit filter (PTFE, 0.22 μm, 5000 rpm, 5 min, r.t.). The recovered filter cake was dried in air overnight. PXRD pattern corresponds to Form A but less crystalline.

### Example 27. R-Ketamine D-Tartrate Salt Form C Scale Up

R-ketamine free base (3.04 g, 12.8 mmol) was dissolved in 21 mL of ethyl acetate at r.t. under stirring. 1.92 g of D-tartaric acid (12.8 mmol, 1 eq) was dissolved in 24 mL of ethanol at r.t. under stirring and sonication treatment. Then the tartaric solution was slowly added to the free base solution under stirring. A cloudy solution was observed after the addition of approx. 2.0 mL of the tartaric solution. The cloudy solution was seeded with R-ketamine D-tartrate salt Form B and R-ketamine D-tartrate salt Form C. The addition was continued and a nice suspension formed. After one day of stirring at r.t., the suspension was filtered over fritted glass filter (porosity 4). The mother liquor was a clear solution. ˜3 mL of the mother liquor was used to flush the reactor. The filter cake was then washed with 10 mL of an ethyl acetate/ethanol 7:8 mixture. The cake was dried on the filter with applied vacuum for 1 hour. Then, further drying was conducted under vacuum (<10 mbar, r.t.). After two days of drying, 4.13 g of white powder was recovered. PXRD pattern corresponds to R-ketamine D-tartrate salt Form C with small less intense reflections at 7°, 8.3°, and 15.2°2-theta. NMR spectrum is consistent with R-ketamine structure. An estimated 1.15 eq of D-tartaric acid was detected at 4.27 ppm, however it should be noted that a broad shoulder is observed from 4 ppm to 6.5 ppm.

### Example 28. Solubility of R-Ketamine D-Tartrate Salt Form C in Water

R-ketamine D-tartrate salt Form C (50 mg) was suspended in 0.5 mL of water. The suspension was shaken at 25° C., 700 rpm. After one hour, pH was measured to be 3.48 at 23° C. After 24 hours of stirring, almost all the material dissolved; only few particles in suspension. pH of 3.42 at 23° C. Almost clear solution. S>100 mg/mL at pH of 3.42 and at 23° C.

### Example 29. Approximate Solubilities of R-Ketamine D-Tartrate Salt Form C

Approximate solubilities were determined for R-ketamine D-tartrate salt Form C and shown below in Table 21. These values were determined by visual inspection after adding small aliquots of solvent or solvent mixture to ˜10 mg of solid and shaking/sonicating for a short period of time at ambient conditions unless specified otherwise. Therefore, it should be noted that these values are only approximations and the error in these data can, in some cases, be rather large. Generally, true thermodynamic values are greater than the kinetic values found using this protocol. The purpose of these measurements is to provide useful information for designing crystallization experiments to discover new forms.

TABLE 21
Solubility data for R-ketamine D-tartrate salt Form C

| Solvent System | Solubility [mg · mL⁻¹] |
|---|---|
| 2-propanol | ~4 |
| Acetone | ~9 |
| Acetonitrile | ~2 |
| Ethanol | ~14 |
| Ethanol at ~40° C. | ~31 |
| Ethyl acetate | <1 |
| Isopropanol | 3~4 |
| Methanol | >92 |
| THF | ~12 |
| Water | 52~104 |

### Example 30. Suspension Experiments Using R-Ketamine D-Tartrate Salt Form C

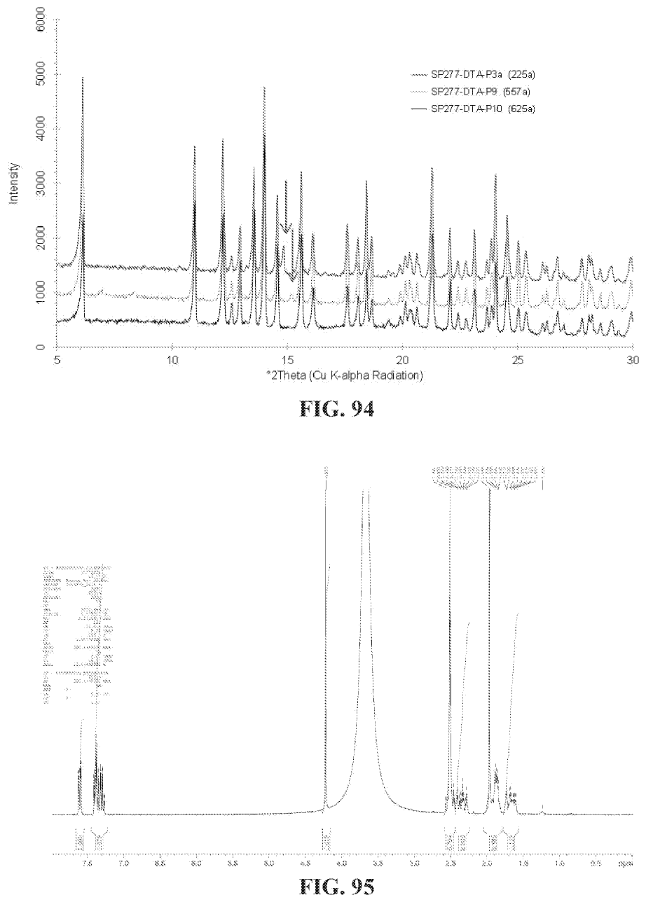

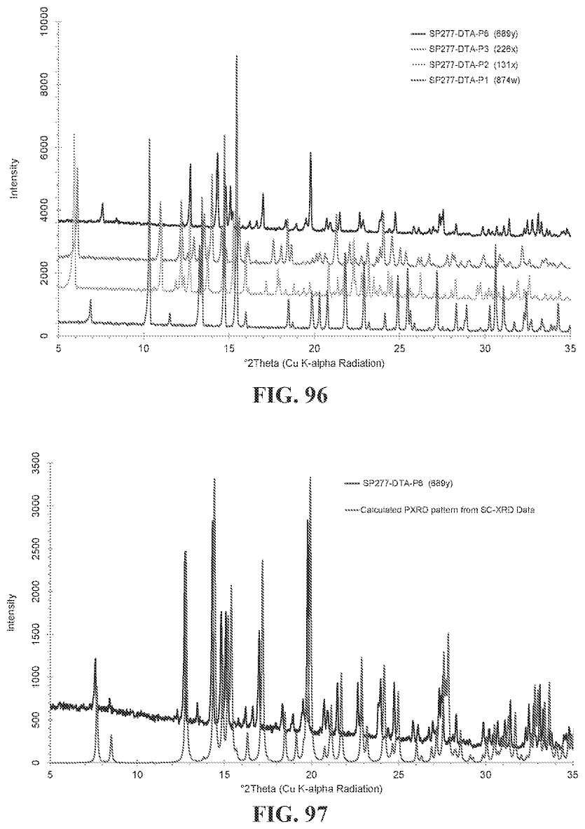

R-ketamine D-tartrate Salt Form C (118.6 mg) was suspended in 1.0 mL of 2-propanol at r.t. A white suspension was obtained. After 1 week of stirring at r.t., the suspension was filtered using a centrifugal unit filter (PVDF, 0.22 μm, 5000 rpm, 5 min, r.t.). The recovered filter cake was dried in air for 30 minutes and then submitted to PXRD. PXRD pattern designated as R-ketamine D-tartrate Salt Form C. Comparison of the PXRD patterns of R-ketamine D-tartrate Salt Form C obtained from Examples 13, 27, and 30 are shown in FIG. 94, showing a peak of the free base form at 14.8° in 2-theta.

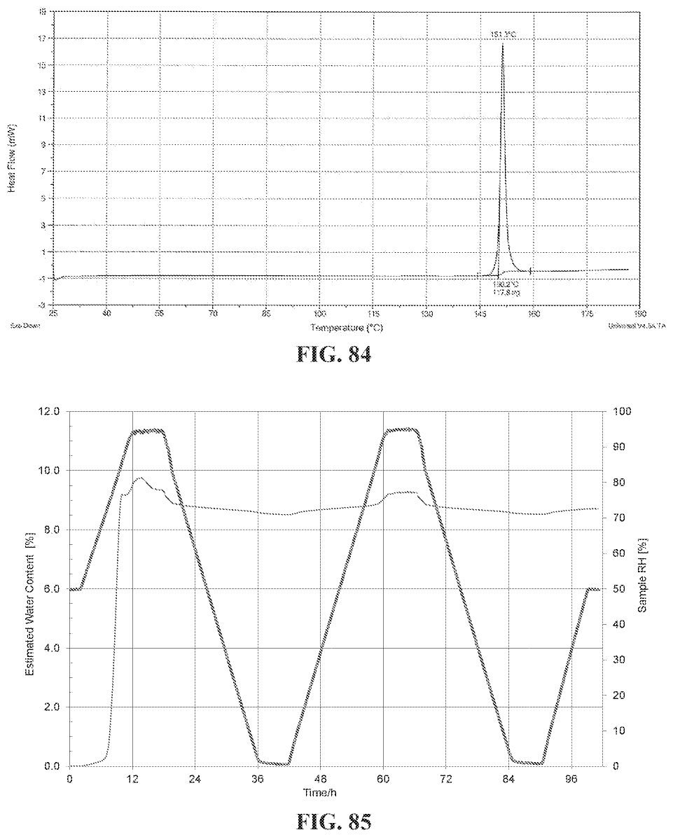

The material was then dried under vacuum (<10 mbar) over the weekend. DSC measurement gave melting peak at 151.3° C., with an onset at 150.2° C., and an enthalpy of fusion of ΔH˜117.8 J/g (FIG. 84).

R-ketamine D-tartrate Salt Form C (123.8 mg) was suspended in 1.0 mL of a 2-propanol/water 98.4/1.6 mixture (water activity ˜0.2) at r.t. A white suspension was obtained. After 1 week of stirring at r.t., the suspension was filtered using a centrifugal unit filter (PVDF, 0.22 μm, 5000 rpm, 5 min, r.t.). The recovered filter cake was dried in air for 30 minutes and then submitted to PXRD. PXRD pattern corresponds to a mixture of R-ketamine D-tartrate Salt Form C and R-ketamine D-tartrate Salt Form D.

R-ketamine D-tartrate Salt Form C (108.7 mg) was suspended in 1.0 mL of a 2-propanol/water 94.7/5.3 mixture (water activity ˜0.5) at r.t. A white suspension was obtained. After 1 week of stirring at r.t., the suspension was filtered using a centrifugal unit filter (PVDF, 0.22 μm, 5000 rpm, 5 min, r.t.). The recovered filter cake was dried in air for 30 minutes and then submitted to PXRD. PXRD pattern corresponds to R-ketamine D-tartrate Salt Form D without small reflections at 12.3°, 13.5°, 16.6°, 20.8°, 33.7°, and 37.2°2theta.

R-ketamine D-tartrate Salt Form C (123.9 mg) was suspended in 1.0 mL of acetone at r.t. A white suspension was obtained. After 1 week of stirring at r.t., the suspension was filtered using a centrifugal unit filter (PTFE, 0.22 μm, 5000 rpm, 5 min, r.t.). The recovered filter cake was dried in air for 30 minutes and then submitted to PXRD. PXRD pattern corresponds to R-ketamine D-tartrate Salt Form C.

### Example 31. Competitive Suspension Experiments with R-Ketamine D-Tartrate Salt Forms B and C

3.0 mL of acetone were added to 101.4 mg of R-ketamine D-tartrate Salt Form C. The obtained suspension was let stir at r.t. for about one hour, then 35 mg of R-ketamine D-tartrate Salt Form B were added. The mixture was placed into a temperature controller and the temperature was set to 2° C., then let stir for one week at 2° C. The suspension was filtered using a centrifugal unit filter (PTFE, 0.22 μm, 4500 rpm, 3 min, r.t.) and the solid powder was dried at r.t. under the fume hood for 15 minutes before being submitted for PXRD. PXRD pattern corresponds to R-ketamine D-tartrate Salt Form C.

4.0 mL of ethanol were added to 197.4 mg of R-ketamine D-tartrate Salt Form C and the mixture was heated to 60° C. A clear solution was quickly and easily obtained and one drop of water (˜15 μL) was added to the clear solution. The solution was filtered into a new glass vial through a syringe filter and left to stand at r.t. After five days, crystalline material was found at the bottom of the vial. The major part of the liquid was collected and the remaining part of the suspension with crystals was left in the open vial to evaporate the solvent under the fume hood for one night. The solid was then collected and submitted for PXRD. PXRD pattern shows a mixture of R-ketamine D-tartrate salt Forms B and C.

To this mixture was added a solvent mixture of 2.0 mL of heptane and 1.0 mL of ethanol. The slurry was kept under stirring at 75° C. overnight in a closed vial. A clear solution was obtained with crystals stuck to the glass wall of the vial. The material was resuspended and let stir at 75° C. in the open vial for 1 hour. Fine crystalline particles where observed, the suspension was then filtered using a centrifugal unit filter (PVDF, 0.22 μm, 4500 rpm, 3 min, r.t.) and the solid powder was then dried at r.t. under the fume hood for 15 minutes before being submitted for PXRD. PXRD corresponds to R-ketamine D-tartrate salt Form C.

R-ketamine D-tartrate Salt Form B (47 mg) and R-ketamine D-tartrate Salt Form C (42.9 mg) were suspended in 1.0 mL of ethyl acetate and the obtained suspension was stirred at r.t. After two days of stirring at 25° C., the suspension was filtered using a centrifugal unit filter (PTFE, 0.22 μm, 5000 rpm, 5 min, r.t.). The recovered filter cake was submitted to PXRD. PXRD pattern corresponds to a mixture of R-ketamine D-tartrate Salt Forms B and C. The material recovered from the PXRD holder was suspended in the recovered mother liquor and further stirred at r.t. After two weeks of stirring at r.t., the half of the suspension was filtered using a centrifugal unit filter (PTFE, 0.22 μm, 5000 rpm, 5 min, r.t.). The recovered filter cake was submitted to PXRD. PXRD pattern corresponds to a mixture of R-ketamine D-tartrate Salt Forms B and C. While the conversion appeared to be very slow, it seemed that R-ketamine D-tartrate salt Form B was disappearing and more R-ketamine D-tartrate salt Form C was being generated.

R-ketamine D-tartrate Salt Form B (60.8 mg) and 60.4 mg of R-ketamine D-tartrate Salt Form C were suspended in 2.0 mL of n-butyl acetate. The suspension was then kept under stirring at 100° C. overnight in a closed vial. The suspension was then filtered using a centrifugal unit filter (PTFE, 0.22 μm, 4500 rpm, 3 min, r.t.) and the solid powder was then dried at r.t. under the fume hood for 15 minutes before being submitted for PXRD. PXRD corresponds to R-ketamine D-tartrate salt Form C.

### Example 32. Precise Solubilities Tests of R-Ketamine Fumarate Salt Form a in Aqueous Media

In this case, pH values close to pH 7 were of interest and pH 5, 6, and 7 were selected for these solubility experiments. It was known from approximate solubility tests, that dissolution of hemifumarate in pure water leads to a pH of about 5.7. For the solubility tests carried out here, R-ketamine fumarate Salt Form A was equilibrated in water and pH adjustments were conducted by addition of 0.1 M or 1 M aqueous NaOH or HCl solutions. The obtained suspensions were equilibrated for one day at 25° C. Filtration was then conducted and the recovered saturated solutions were submitted for HPLC. The results are summarized in Table 22 and shows that the solubility rapidly deceases by increasing pH. PXRD of the solid residues obtained after filtration showed that the solid phases converted to the free base form during equilibration.

TABLE 22
Aqueous solubility data for R-ketamine fumarate salt Form A at r.t.

| pH | Solubility [mg/mL] |
|---|---|
| 5.23 | 304 |
| 6.14 | 15.1 |
| 7.21 | 1.76 |

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. An R-ketamine saccharin salt.

2. The R-ketamine saccharin salt of claim 1, wherein the saccharin salt is crystalline.

3. The R-ketamine saccharin salt of claim 1, wherein the saccharin salt is amorphous.

4. The R-ketamine saccharin salt of claim 1 or 2, wherein the R-ketamine saccharin salt is a crystalline polymorphic form characterized by PXRD peaks at two or more, or three peaks selected from the group consisting of 13.8 °2θ, 15.6 °2θ, and 23.5 °2θ (±0.2 °2θ; ±0.1 °2θ; or ±0.0 °2θ; Cu Kα1 radiation).

5. The R-ketamine saccharin salt of claim 1, 2, or 4, wherein the R-ketamine saccharin salt is a crystalline polymorphic form characterized by PXRD peaks at 13.8 °2θ, 15.6 °2θ, and 23.5 °2θ (±0.2 °2θ; ±0.1 °2θ; or ±0.0 °2θ; Cu Kα1 radiation).

6. The R-ketamine saccharin salt of any one of claims 1, 2, 4, and 5, wherein the R-ketamine saccharin salt is a crystalline polymorphic form characterized by a PXRD spectrum substantially similar to that shown in FIG. 9.

7. The R-ketamine saccharin salt of any one of claims 1, 2, and 4-6, wherein the R-ketamine saccharin salt is a crystalline polymorphic form characterized by one, two, three, four, five, six, seven, eight, nine, ten, eleven, twelve, thirteen, fourteen, fifteen or sixteen PXRD peaks selected from those set forth in in Table 2.

8. The R-ketamine saccharin salt of any one of claims 1-7, wherein the R-ketamine saccharin salt is a 1:1 R-ketamine:saccharin salt.

9. The R-ketamine saccharin salt of any one of claims 1-7, wherein the R-ketamine saccharin salt is a 2:1 R-ketamine:saccharin salt.

10. The R-ketamine saccharin salt of any one of claims 1-7, wherein the R-ketamine saccharin salt is a 1:2 R-ketamine:saccharin salt.

11. An R-ketamine fumarate salt.

12. The R-ketamine fumarate salt of claim 11, wherein the fumarate salt is crystalline.

13. The R-ketamine fumarate salt of claim 11 or 12, wherein the R-ketamine fumarate salt is Form A.

14. The R-ketamine fumarate salt Form A of claim 13, characterized by PXRD peaks at two or more, or three peaks selected from the group consisting of 11.6 °2θ, 13.4 °2θ, and 14.6 °2θ (±0.2 °2θ; ±0.1 °2θ; or ±0.0 °2θ; Cu Kα1 radiation).

15. The R-ketamine fumarate salt Form A of claim 13 or 14, characterized by PXRD peaks at 11.6 °2θ, 13.4 °2θ, and 14.6 °2θ (±0.2 °2θ; ±0.1 °2θ; or 0.0 °2θ; Cu Kα1 radiation).

16. The R-ketamine fumarate salt Form A of any one of claims 13-15, characterized by a PXRD spectrum substantially similar to that shown in FIG. 21.

17. The R-ketamine fumarate salt Form A of any one of claims 13-16, characterized by one, two, three, four, five, six, seven, eight, nine, ten, eleven, twelve, thirteen, fourteen, fifteen or sixteen PXRD peaks selected from those set forth in Table 3.

18. The R-ketamine fumarate salt of claim 11 or 12, wherein the R-ketamine fumarate salt is Form B.

19. The R-ketamine fumarate salt Form B of claim 18, characterized by PXRD peaks at two or more, or three peaks selected from the group consisting of 14.5 °2θ, 15.1 °2θ, and 20.4 °2θ (±0.2 °2θ; ±0.1 °2θ; or ±0.0 °2θ; Cu Kα1 radiation).

20. The R-ketamine fumarate salt Form B of claim 18 or claim 19, characterized by PXRD peaks at 14.5 °2θ, 15.1 °2θ, and 20.4 °2θ (±0.2 °2θ; ±0.1 °2θ; or ±0.0 °2θ; Cu Kα1 radiation).

21. The R-ketamine fumarate salt Form B of any one of claims 18-20, characterized by a PXRD spectrum is substantially similar to that shown in FIG. 68.

22. The R-ketamine fumarate salt Form B of any one of claims 18-21, characterized by one, two, three, four, five, six, seven, eight, nine, ten, eleven, twelve, thirteen, fourteen, fifteen or sixteen PXRD peaks selected from those set forth in Table 4.

23. The R-ketamine fumarate salt according to claims 11-22, wherein the R-ketamine fumarate salt is a 1:1 R-ketamine:fumarate salt.

24. The R-ketamine fumarate salt according to claims 11-22, wherein the R-ketamine fumarate salt is a 2:1 R-ketamine:fumarate salt.

25. The R-ketamine fumarate salt according to claims 11-22, wherein the R-ketamine fumarate salt is between a 1:1 and 1:2 R-ketamine:fumarate salt.

26. An R-ketamine succinate salt.

27. The R-ketamine succinate salt of claim 26, wherein the succinate salt is crystalline.

28. The R-ketamine succinate salt of claim 26 or 27, wherein the R-ketamine succinate salt is a crystalline polymorphic form characterized by PXRD peaks at two or more, or three peaks selected from the group consisting of 9.0 °2θ, 13.5 °2θ, and 18.1 °2θ (±0.2 °2θ; ±0.1 °2θ; or ±0.0 °2θ; Cu Kα1 radiation).

29. The R-ketamine succinate salt of any one of claims 26-28, wherein the R-ketamine succinate salt is a crystalline polymorphic form characterized by PXRD peaks at 9.0 °2θ, 13.5 °2θ, and 18.1 °2θ (±0.2 °2θ; ±0.1 °2θ; or ±0.0 °2θ; Cu Kα1 radiation).

30. The R-ketamine succinate salt of any one of claims 26-29, wherein the R-ketamine succinate salt is a crystalline polymorphic form characterized by a PXRD spectrum substantially similar to that shown in FIG. 29.

31. The R-ketamine succinate salt of claim 28, wherein the R-ketamine succinate salt is a crystalline polymorphic form characterized by one, two, three, four, five, six, seven, eight, nine, ten, eleven, twelve, thirteen, fourteen, fifteen or sixteen PXRD peaks selected from those set forth in Table 5.

32. An R-ketamine sulfate salt.

33. The R-ketamine sulfate salt of claim 32, wherein the sulfate salt is crystalline.

34. The R-ketamine sulfate salt of claim 32 or 33, wherein the R-ketamine sulfate salt is a crystalline polymorphic form characterized by PXRD peaks at two or more, or three peaks selected from the group consisting of 19.8 °2θ, 22.5 °2θ, and 26.1 °2θ (±0.2 °2θ; ±0.1 °2θ; or ±0.0 °2θ; Cu Kα1 radiation).

35. The R-ketamine sulfate salt of any one of claims 32-34, wherein the R-ketamine sulfate salt is a crystalline polymorphic form characterized by PXRD peaks at 19.8 °2θ, 22.5 °2θ, and 26.1 °2θ (±0.2 °2θ; ±0.1 °2θ; or ±0.0 °2θ; Cu Kα1 radiation).

36. The R-ketamine sulfate salt of any one of claims 32-35, wherein the R-ketamine sulfate salt is a crystalline polymorphic form characterized by a PXRD spectrum substantially similar to that shown in FIG. 36.

37. The R-ketamine sulfate salt of any one of claims 32-36, wherein the R-ketamine sulfate salt is a crystalline polymorphic form characterized by one, two, three, four, five, six, seven, eight, nine, or ten PXRD peaks selected from those set forth in Table 6.

38. An R-ketamine D-tartrate salt.

39. The R-ketamine D-tartrate salt of claim 38, wherein the D-tartrate salt is crystalline.

40. The R-ketamine D-tartrate salt of claim 38 or 39, wherein the R-ketamine D-tartrate salt is Form A.

41. The R-ketamine D-tartrate salt Form A of claim 40, characterized by PXRD peaks at two or more, or three peaks selected from the group consisting of 10.3 °2θ, 14.7 °2θ, and 15.4 °2θ (±0.2 °2θ; ±0.1 °2θ; or ±0.0 °2θ; Cu Kα1 radiation).

42. The R-ketamine D-tartrate salt Form A of claim 40 or 41, characterized by PXRD peaks at 10.3 °2θ, 14.7 °2θ, and 15.4 °2θ (±0.2 °2θ; ±0.1 °2θ; or 0.0 °2θ; Cu Kα1 radiation).

43. The R-ketamine D-tartrate salt Form A of any one of claims 40-42, characterized by a PXRD spectrum substantially similar to that shown in FIG. 40.

44. The R-ketamine D-tartrate salt Form A of any one of claims 40-43, characterized by one, two, three, four, five, six, seven, eight, nine, ten, eleven, twelve, thirteen, fourteen, fifteen or sixteen PXRD peaks selected from those set forth in Table 7.

45. The R-ketamine D-tartrate salt of claim 38 or 39, wherein the R-ketamine D-tartrate salt is Form B.

46. The R-ketamine D-tartrate salt Form B of claim 45, characterized by PXRD peaks at two or more, or three peaks selected from the group consisting of 5.9 °2θ, 12.7 °2θ, and 14.7 °2θ (±0.2 °2θ; ±0.1 °2θ; or ±0.0 °2θ; Cu Kα1 radiation).

47. The R-ketamine D-tartrate salt Form B of claim 45 or 46, characterized by PXRD peaks at 5.9 °2θ, 12.7 °2θ, and 14.7 °2θ (±0.2 °2θ; ±0.1 °2θ; or ±0.0 °2θ; Cu Kα1 radiation).

48. The R-ketamine D-tartrate salt Form B of any one of claims 45-47, characterized by a PXRD spectrum substantially similar to that shown in FIG. 56.

49. The R-ketamine D-tartrate salt Form B of any one of claims 45-48, characterized by one, two, three, four, five, six, seven, eight, nine, ten, eleven, twelve, thirteen, fourteen, fifteen or sixteen PXRD peaks selected from those set forth in Table 8.

50. The R-ketamine D-tartrate salt of claim 38 or 39, wherein the R-ketamine D-tartrate salt is Form C.

51. The R-ketamine D-tartrate salt Form C of claim 50, characterized by PXRD peaks at two or more, or three peaks selected from the group consisting 6.1 °2θ, 11.0 °2θ, and 12.2 °2θ (±0.2 °2θ; ±0.1 °2θ; or ±0.0 °2θ; Cu Kα1 radiation).

52. The R-ketamine D-tartrate salt Form C of claim 50 or 51, characterized by PXRD peaks at 6.1 °2θ, 11.0 °2θ, and 12.2 °2θ (±0.2 °2θ; ±0.1 °2θ; or ±0.0 °2θ; Cu Kα1 radiation).

53. The R-ketamine D-tartrate salt Form C of any one of claims 50-52, characterized by a PXRD spectrum substantially similar to that shown in FIG. 61.

54. The R-ketamine D-tartrate salt Form C of any one of claims 50-53, characterized by one, two, three, four, five, six, seven, eight, nine, ten, eleven, twelve, thirteen, fourteen, fifteen or sixteen PXRD peaks selected from those set forth in Table 9.

55. The R-ketamine D-tartrate salt according to any one of claims 38-54, wherein the R-ketamine D-tartrate salt is a 1:1 R-ketamine:D-tartrate salt.

56. The R-ketamine D-tartrate salt according to any one of claims 38-54, wherein the R-ketamine D-tartrate salt is a 2:1 R-ketamine:D-tartrate salt.

57. The R-ketamine D-tartrate salt according to any one of claims 38-54, wherein the R-ketamine D-tartrate salt is between a 1:1 and a 2:1 R-ketamine:D-tartrate salt.

58. An R-ketamine oxalate salt.

59. The R-ketamine oxalate salt of claim 58, wherein the oxalate salt is crystalline.

60. The R-ketamine oxalate salt of claim 58 or 59, wherein the R-ketamine oxalate salt is a crystalline polymorphic form characterized by PXRD peaks at two or more, or three peaks selected from the group consisting of 12.8 °2θ, 14.8 °2θ and 16.2 °2θ (10.2 °2θ; ±0.1 °2θ; or ±0.0 °2θ; Cu Kα1 radiation).

61. The R-ketamine oxalate salt of any one of claims 58-60, wherein the R-ketamine oxalate salt is a crystalline polymorphic form characterized by PXRD peaks at 12.8 °2θ, 14.8 °2θ and 16.2 °2θ (±0.2 °2θ; ±0.1 °2θ; or ±0.0 °2θ; Cu Kα1 radiation).

62. The R-ketamine oxalate salt of any one of claims 58-61, wherein the R-ketamine oxalate salt is a crystalline polymorphic form characterized by a PXRD spectrum substantially similar to that shown in FIG. 43.

63. The R-ketamine oxalate salt of any one of claims 59-62, wherein the R-ketamine oxalate salt is a crystalline polymorphic form characterized by one, two, three, four, five, six, seven, eight, nine, ten, eleven, twelve, thirteen, fourteen, fifteen or sixteen PXRD peaks selected from those set forth in Table 12.

64. The R-ketamine oxalate salt of any one of claims 59-63, wherein the R-ketamine oxalate salt is a 2:1 R-ketamine:oxalate salt.

65. An R-ketamine citrate salt.

66. The R-ketamine citrate salt of claim 65, wherein the citrate salt is crystalline.

67. The R-ketamine citrate salt of claim 65 or 66, wherein the R-ketamine citrate salt is a crystalline polymorphic form characterized by PXRD peaks at two or more, or three peaks selected from the group consisting of 14.8 °2θ, 16.8 °2θ, and 21.4 °2θ (±0.2 °2θ; ±0.1 °2θ; or ±0.0 °2θ; Cu Kα1 radiation).

68. The R-ketamine citrate salt of any one of claim 65-67, wherein the R-ketamine citrate salt is a crystalline polymorphic form characterized by PXRD peaks at 14.8 °2θ, 16.8 °2θ, and 21.4 °2θ (±0.2 °2θ; ±0.1 °2θ; or ±0.0 °2θ; Cu Kα1 radiation).

69. The R-ketamine citrate salt of any one of claims 65-68, wherein the R-ketamine citrate salt is a crystalline polymorphic form characterized by a PXRD spectrum substantially similar to that shown in FIG. 46.

70. The R-ketamine oxalate salt of any one of claims 65-69, wherein the R-ketamine citrate salt is a crystalline polymorphic form characterized by one, two, three, four, five, six, seven, eight, nine, ten, eleven, twelve, thirteen, fourteen, fifteen or sixteen PXRD peaks selected from those set forth in Table 12.

71. The R-ketamine oxalate salt of any one of claims 65-70, wherein the R-ketamine citrate salt is a 1:1 R-ketamine:citrate salt.

72. The R-ketamine citrate salt of any one of claims 65-70, wherein the R-ketamine citrate salt is a 2:1 R-ketamine:citrate salt.

73. The R-ketamine citrate salt of any one claims 65-72, wherein the R-ketamine citrate salt is between a 1:1 and a 3:1 R-ketamine:citrate salt.

74. An R-ketamine free base Form A.

75. The R-ketamine free base Form A of claim 74, wherein the R-ketamine free base Form A is crystalline.

76. The R-ketamine free base of claim 74 or 75, wherein the R-ketamine free base Form A is characterized by PXRD peaks at two or more, or three peaks selected from the group consisting of 14.8 °2θ, 20.7 °2θ, and 23.8 °2θ (±0.2 °2θ; ±0.1 °2θ; or ±0.0 °2θ; Cu Kα1 radiation).

77. The R-ketamine free base of any one of claims 74-76, wherein R-ketamine free base Form A is characterized by PXRD peaks at 14.8 °2θ, 20.7 °2θ, and 23.8 °2θ (±0.2 °2θ; ±0.1 °2θ; or ±0.0 °2θ; Cu Kα1 radiation).

78. The R-ketamine free base of any one of claims 74-77, wherein R-ketamine free base Form A is characterized by a PXRD spectrum substantially similar to that shown in FIG. 1.

79. The R-ketamine free base of any one of claims 74-78, wherein R-ketamine free base Form A is characterized by one, two, three, four, five, six, seven, eight, nine, ten, eleven, twelve, thirteen, fourteen, fifteen or sixteen PXRD peaks set forth in Table 1.

80. The R-ketamine free base of any one of claims 74-79, wherein R-ketamine free base Form A is characterized by an FT-Raman spectrum substantially similar to that shown in FIG. 3.

81. The R-ketamine free base of any one of claims 74-80, wherein R-ketamine free base Form A is characterized by a ¹H-NMR spectrum substantially similar to that shown in FIG. 6.

82. The R-ketamine D-tartrate salt Form C of any one of claims 50-53, characterized by one, two, three, four, five, six, seven, eight, nine, ten, eleven, twelve, thirteen, fourteen, fifteen or sixteen PXRD peaks selected from those set forth in Table 10.

83. The R-ketamine D-tartrate salt of either claim 38 or 39, wherein the R-ketamine D-tartrate salt is Form D.

84. The R-ketamine D-tartrate salt Form D of claim 83, characterized by PXRD peaks at two or more, or three peaks selected from the group consisting 12.7 °2θ, 14.3 °2θ, and 19.8 °2θ (±0.2 °2θ; ±0.1 °2θ; or ±0.0 °2θ; Cu Kα1 radiation).

85. The R-ketamine D-tartrate salt Form D of claim 83 or 84, characterized by PXRD peaks at 12.7 °2θ, 14.3 °2θ, and 19.8 °2θ (±0.2 °2θ; ±0.1 °2θ; or ±0.0 °2θ; Cu Kα1 radiation).

86. The R-ketamine D-tartrate salt Form D of any one of claims 83-85, characterized by a PXRD spectrum substantially similar to that shown in FIG. 87.

87. The R-ketamine D-tartrate salt Form D of any one of claims 83-86, characterized by one, two, three, four, five, six, seven, eight, nine, ten, eleven, twelve, thirteen, fourteen, fifteen or sixteen PXRD peaks selected from those set forth in Table 11.

88. The R-ketamine D-tartrate salt according to any one of claims 38-54 or 82-86, wherein the R-ketamine D-tartrate salt is a 1:1 R-ketamine:D-tartrate salt.

89. The R-ketamine D-tartrate salt according to any one of claims 38-54 or 82-86, wherein the R-ketamine D-tartrate salt is a 2:1 R-ketamine:D-tartrate salt.

90. The R-ketamine D-tartrate salt according to any one of claims 38-54 or 82-86, wherein the R-ketamine D-tartrate salt is between a 1:1 and a 2:1 R-ketamine:D-tartrate salt.

91. A pharmaceutical composition comprising one or more of the salts or salt forms according to any one of claims 1-90 and a pharmaceutically acceptable excipient, diluent or carrier.

92. A pharmaceutical composition comprising one or more of the salts or salt forms according to any one of claims 1-90 for use in the treatment or prevention of depression or depressive symptoms in a patient in need thereof.

93. Use of the salts or salt forms according to any one of claims 1-90 in the preparation of a medicament for the treatment or prevention of depression or depressive symptoms in a patient in need thereof.

94. A method of treating or preventing depression or depressive symptoms comprising administering a salt, salt form, or pharmaceutical composition according to any one of claims 1-81 to a patient in need thereof.

95. The composition of either claim 91 or 92, use of claim 93, or method of claim 94, wherein the depression or depressive symptom is treatment resistant depression.
